(19) United States
(12) Patent Application Publication
(10) Pub. No.: US 2026/0226045 A1
(43) Pub. Date: Aug. 6, 2026

(54) PROCESSES AND INTERMEDIATES FOR SYNTHESIS OF ADAGRASIB

(71) Applicant: MIRATI THERAPEUTICS, INC., PRINCETON, NJ (US)

(72) Inventors: THOMAS SCATTOLIN, SAN DIEGO, CA (US); CHENG CHEN, SAN DIEGO, CA (US); YONGHONG GAN, SAN DIEGO, CA (US); CHENGSHENG CHEN, LA JOLLA, CA (US); ZHICHAO LU, SAN DIEGO, CA (US)

(73) Assignee: MIRATI THERAPEUTICS, INC., PRINCETON, NJ (US)

(21) Appl. No.: 19/152,025

(22) PCT Filed: Jan. 31, 2024
(86) PCT No.: PCT/US2024/013794

§ 371 Date: Jul. 30, 2025

Related U.S. Application Data
(60) Provisional application No. 63/442,400, filed on Jan. 31, 2023.

(51) Int. Cl. C07D 471/04; C07C 227/06; C07C 229/20; C07C 259/06; C07D 211/78

(52) U.S. Cl. CPC C07D 471/04; C07C 227/06; C07C 229/20; C07C 259/06; C07D 211/78

(57) ABSTRACT

The present invention relates to new synthetic routes of synthesizing adagrasib. The invention also provides intermediates used in the provided synthetic routes.

### FIELD OF THE INVENTION

The present invention relates to new and improved synthetic routes for synthesis of adagrasib.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors regulating a wide variety of processes, including cellular proliferation (e.g., see Alamgeer et al., (2013) Current Opin Pharmcol. 13:394-401).

The role of activated KRas in malignancy was observed over thirty years ago (e.g., see Der et al., (1982) Proc. Natl Acad. Sci. USA 79(11):3637-3640). Aberrant expression of KRas accounts for up to 20% of all cancers and oncogenic KRas mutations that stabilize GTP binding and lead to constitutive activation of KRas and downstream signaling have been reported in 25-30% of lung adenocarcinomas. (e.g., see Samatar and Poulikakos (2014) Nat Rev Drug Disc 13(12): 928-942 doi: 10.1038/nrd428). Single nucleotide substitutions that result in missense mutations at codons 12 and 13 of the KRas primary amino acid sequence comprise approximately 40% of these KRas driver mutations in lung adenocarcinoma, with a G12C transversion being the most common activating mutation (e.g., see Dogan et al., (2012) Clin Cancer Res. 18(22):6169-6177, published online 2012 Sep. 26. doi: 10.1158/1078-0432.CCR-11-3265).

The well-known role of KRas in malignancy and the discovery of these frequent mutations in KRas in various tumor types made KRas a highly attractable target of the pharmaceutical industry for cancer therapy. Notwithstanding thirty years of large scale discovery efforts to develop inhibitors of KRas for treating cancer, no KRas inhibitor has demonstrated sufficient safety and/or efficacy to obtain regulatory approval (e.g., see McCormick (2015) Clin Cancer Res. 21 (8):1797-1801).

KRas G12C inhibitor compound 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (also known as MRTX849, and also known as adagrasib) has the following structure:

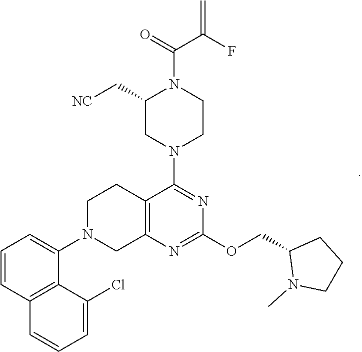 [SMILES: C=C(F)C(=O)N1CCN(c2nc(OC[C@@H]3CCCN3C)nc3c2CCN(c2cccc4cccc(Cl)c24)C3)C[C@@H]1CC#N ; "."]

Adagrasib is described, for example, in Example 478 of PCT Application WO 2019/099524.

While WO 2019/099524 describes methods of making adagrasib, there is a need in the art for new and improved synthetic routes of making adagrasib.

### SUMMARY OF THE INVENTION

The present invention, in one embodiment, provides new and improved methods of making adagrasib.

In one embodiment, the invention provides a method of synthesizing adagrasib, comprising step (a):
a) reacting a compound of the following structure:

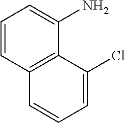 [SMILES: Nc1cccc2cccc(Cl)c12]

with a 4-halobutyrate, a aprotic solvent, an iodide, and a base to produce a final compound of step (a) with the following structure:

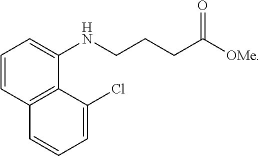 [SMILES: COC(=O)CCCNc1cccc2cccc(Cl)c12]

In one embodiment, step (a) is carried out at a temperature from about 20° C. to about 120° C.

In one embodiment, the 4-halobutyrate is 4-X(CH₂)₃CO₂R, wherein R is any alkyl or (hetero)aryl group selected from the group consisting of methyl, ethyl, propyl, and trifluoroethyl, and wherein X is any leaving group. In one embodiment, X is selected from the group consisting of Cl, Br, I, MsO, TsO, and TfO.

In one embodiment, the aprotic solvent is selected from the group consisting of toluene, anisole, xylene, dioxane, dichloromethane, dimethylformamide, dimethylacetamide, acetonitrile, 2-methyltetrahydrofuran, tetrahydrofuran, cyclopentyl methyl ether, methyl tert-butyl ether, and diethylene glycol dimethyl.

In one embodiment, the iodide is selected from the group consisting of sodium iodide, potassium iodide, and alkylated ammonium iodide.

In one embodiment, the base is an organic base.

In one embodiment, the organic base is selected from the group consisting of Diisopropylethylamine (DIPEA), triethylamine (Et₃N), triethylenediamine (DABCO), and 1,8-Diazabicyclo[5.4.0]undec-7-ene (DBU).

In another embodiment, the base is an inorganic base.

In one embodiment, the inorganic base is selected from the group consisting of carbonate, bicarbonate, and phosphate. An inorganic base can be used with any alkali such as lithium, sodium and potassium.

In one embodiment, the method further comprises step (b):
b) reacting the final compound of step (a) with a 2-halo-N-methoxy-N-methylacetamide, an aprotic solvent and a base to produce a final compound of step (b) with the following structure:

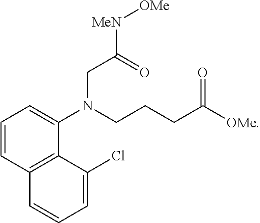 [SMILES: COC(=O)CCCN(CC(=O)N(C)OC)c1cccc2cccc(Cl)c12]

In one embodiment, step (b) is carried out at a temperature from about 20° C. to about 150° C.

In one embodiment, the 2-halo-N-methoxy-N-methylacetamide is XCH₂C(O)NMeOMe wherein X is selected from the group consisting of Cl, Br, I, MsO, TsO, and TfO.

In one embodiment, the aprotic solvent is selected from the group consisting of toluene, anisole, xylene, diethylene glycol dimethyl, DMAc, DMF, 1,4-dioxane, THF, 2-MeTHF, MeCN, DMSO, and NMP.

In one embodiment, step (b) further comprises an iodide.

In one embodiment, the iodide is selected from the group consisting of sodium iodide, potassium iodide, and alkylated ammonium iodide.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, and phosphate. An inorganic base can be used with any alkali such as lithium, sodium and potassium.

In one embodiment, the method further comprises step (c):
c) reacting the final compound of step (b) with a base and an aprotic solvent to produce a final compound of step (c) with the following structure:

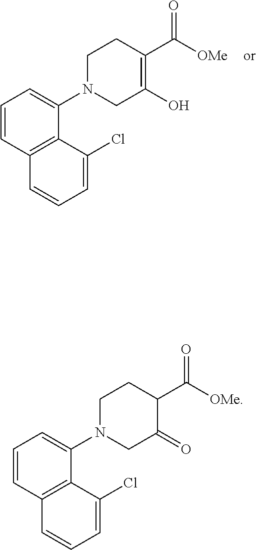 [SMILES: COC(=O)C1=C(O)CN(c2cccc3cccc(Cl)c23)CC1 ; COC(=O)C1CCN(c2cccc3cccc(Cl)c23)CC1=O]

In one embodiment, step (c) is carried out at a temperature from about −80° C. to about 25° C.

In one embodiment, the base is selected from the group consisting of lithium bis(trimethylsilyl)amide (LiHMDS), sodium bis(trimethylsilyl)amide (NaHMDS), potassium bis(trimethylsilyl)amide (KHMDS), lithium diisopropylamide (LDA), and lithium tetramethylpiperidide (LiTMP).

In one embodiment, the aprotic solvent is selected from the group consisting of toluene, anisole, xylene, diethylene glycol dimethyl, DMAc, DMF, 1,4-dioxane, THF, 2-MeTHF, MeCN, DMSO, and NMP.

In one embodiment, the method further comprises step (d):
d) reacting the final compound of step (c) with an S-alkylated isothiourea salt, a solvent and a base to produce a final compound of step (d) with the following structure:

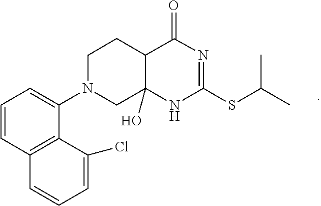 [SMILES: CC(C)SC1=NC(=O)C2CCN(c3cccc4cccc(Cl)c34)CC2(O)N1]

In one embodiment, step (d) is carried out at a temperature from about −20° C. to about 50° C.

In one embodiment, an alkyl group in the S-alkylated isothiourea salt is selected from the group consisting of methyl, ethyl, n-propyl, 2-propyl, cyclopropyl, n-butyl, i-butyl, s-butyl, t-butyl, cyclobutyl, n-pentyl, i-pentyl, s-pentyl, t-pentyl, cyclopentyl, n-hexyl, i-hexyl, s-hexyl, t-hexyl, cyclohexyl, and benzyl, and further wherein a counterion is selected from the group consisting of Cl⁻, Br⁻, I⁻, MsO⁻, TsO⁻, TfO⁻, BF₄⁻, SbF₆⁻, CF₃COO⁻, NO₃⁻· and SO₄²⁻.

In one embodiment, the solvent is an alcoholic solvent.

In one embodiment, the solvent is selected from the group consisting of DMAc, DMF, 1,4-dioxane, THF, 2-MeTHF, MeCN, DMSO, DCM, EtOAc, IPAc, and NMP.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the method further comprises step (e):
e) reacting the final compound of step (d) with an acid, a triflating/mesylating agent and an aprotic solvent to produce a final compound of step (e) with the following structure:

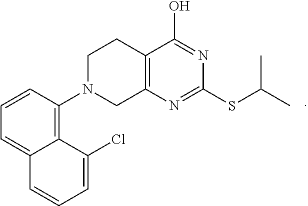 [SMILES: CC(C)Sc1nc(O)c2c(n1)CN(c1cccc3cccc(Cl)c13)CC2]

In one embodiment, step (e) is carried out at a temperature from about 0° C. to about [symbol] ° C.

In one embodiment, the aprotic solvent is selected from the group consisting of toluene, anisole, xylene, diethylene glycol dimethyl, DMAc, DMF, 1,4-dioxane, THF, 2-MeTHF, MeCN, DMSO, and NMP.

In one embodiment, the acid is selected from the group consisting of trifluoroacetic acid, triflic acid, methanesulfonic acid, sulfuric acid and HCl.

In one embodiment, the triflating agent is selected from the group consisting of Tf₂O, CF₃SO₂Cl, and CF₃SO₂Br. The mesylating agent can comprise, but is not limited to, MsCl or (MeSO₂)₂O.

In one embodiment, the method further comprises step (f):
f) reacting the final compound of step (e) with an oxidizing agent, a base and/or an alkoxide, a polar solvent and, optionally, a catalyst to produce a final compound of step (f) with the following structure:

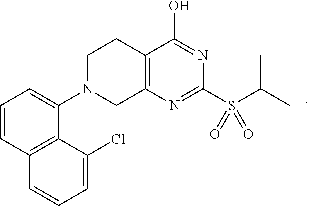 [SMILES: CC(C)S(=O)(=O)c1nc(O)c2c(n1)CN(c1cccc3cccc(Cl)c13)CC2]

In one embodiment, step (f) is carried out at about −15° C. to about 60° C.

In one embodiment, the oxidizing agent is selected from the group consisting of peracid, oxone, bleach, hydrogen peroxide, NaIO₄, perborate, percarbonate and urea hydrogen peroxide.

In one embodiment, the oxidizing agent is hydrogen peroxide.

In one embodiment, the catalyst is selected from the group consisting of sodium tungstate, phenylphosphonic acid, and methyltrioctylammonium hydrogensulfate.

In one embodiment, the alkoxide is selected from the group consisting of methoxide, ethoxide, iso-propoxide, tert-butoxide and tert-amylate, or ammonium or alkali salts thereof.

In one embodiment, the base is an inorganic base.

In one embodiment, the inorganic base is selected from the group consisting of carbonate, bicarbonate, phosphate, and alkali salts thereof.

In one embodiment, the polar solvent is selected from the group consisting of acetonitrile and ROH, wherein R is methyl, ethyl, or 2-propyl.

In one embodiment, the invention further comprises step (g):
g) reacting the final compound of step (f) with an alkoxide and a polar aprotic solvent to produce a final compound of step (g) with the following structure:

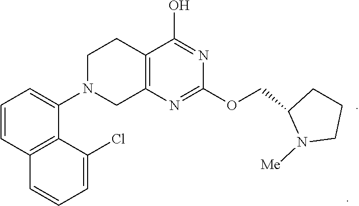 [SMILES: CN1CCC[C@H]1COc1nc(O)c2c(n1)CN(c1cccc3cccc(Cl)c13)CC2]

In one embodiment, step (g) is carried out at about −20° C. to about 100° C.

In one embodiment, the alkoxide is a bulky alkoxide. As used herein, the term “bulky alkoxide” means a sterically hindered alkoxide that is a poor nucleophile.

In one embodiment, the alkoxide is selected from the group consisting of iso-propoxide, tert-butoxide, tert-amylate, and alkali salts thereof.

In one embodiment, the polar aprotic solvent is selected from the group consisting of DMAc, DMF, 1,4-dioxane, THF, 2-MeTHF, MeCN, DMSO, 2-propanol and NMP.

In one embodiment, the invention further comprises step (h):
h) reacting the final compound of step (g) with an activating agent, a base, a polar aprotic solvent and an additive, to produce a final compound of step (h) with the following structure:

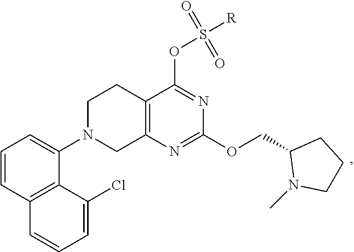

wherein R is selected from the group consisting of a substituted phenyl, methyl, ethyl, propyl, C₄F₉, and toluene.

In one embodiment, step (h) is carried out at a temperature from about −80° C. to about 120° C.

In one embodiment, the activating agent is selected from the group consisting of sulfonyl halide R—SO₂X (wherein R is tolyl, mesityl, nosyl, methyl, ethyl, or propyl and X is F, Cl, Br, Oms, or OTs), anhydride, and organic triflate reagent R¹—N-Tf₂ (where R¹ is phenyl, 5-chloro-2-pyridine, or 2-pyridine).

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the polar aprotic solvent is selected from the group consisting of DMAc, DMF, 1,4-dioxane, THF, 2-MeTHF, MeCN, DMSO, 2-propanol and NMP.

In one embodiment, the additive is selected from the group consisting of pyridine and a substituted pyridine.

In one embodiment, the invention further comprises step (i):
i) reacting the final compound of step (h) with a salt of

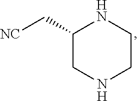

a base and a polar aprotic solvent to produce a final compound of step (i) with the following structure:

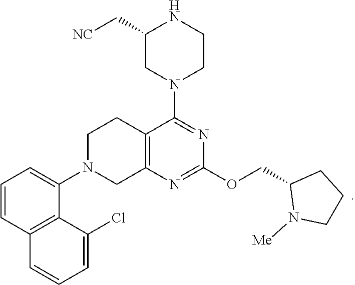 [SMILES: CN1CCC[C@H]1COc1nc2c(c(N3CCN[C@@H](CC#N)C3)n1)CCN(c1cccc3cccc(Cl)c13)C2]

In one embodiment, step (i) is carried out at a temperature from about 0° C. to about 100° C.

In one embodiment, the salt is selected from the group consisting of HCl, TFA and HBr.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the polar aprotic solvent is selected from the group consisting of DMAc, DMF, 1,4-dioxane, THF, 2-MeTHF, MeCN, DMSO, 2-propanol and NMP.

In one embodiment, the invention further comprises step (j):
j) reacting the final compound of step (i) with a salt of 2-fluoroacrylic acid, a solvent and optionally a base to produce adagrasib.

In one embodiment, step (j) is carried out at a temperature from about −10° C. to about 50° C.

In one embodiment, the salt is a lithium, sodium, potassium, or ammonium salt.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the solvent is selected from the group consisting of DMAc, DMF, 1,4-dioxane, THF, 2-MeTHF, MeCN, DMSO, NMP, and an alcohol with a formula R—OH, wherein R is alkyl, allyl or aryl.

In one embodiment, the steps (d), (e), and (f) described above may be replaced by the following steps (d′), (e′) and (f′) to provide an alternative route to synthesize the final compound of step (f).
d′) reacting the final compound of step (c) with an alkoxide and a polar solvent to produce a final compound of step (d′) with the following structure:

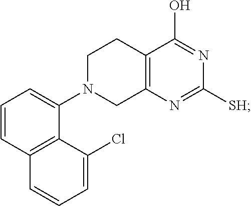 [SMILES: Oc1nc(S)nc2c1CCN(c1cccc3cccc(Cl)c13)C2]

e′) reacting the final compound of step (d′) with an alkylating agent, an inorganic base and/or an alkoxide, and a polar solvent to produce a final compound of step (e′) with the following structure:

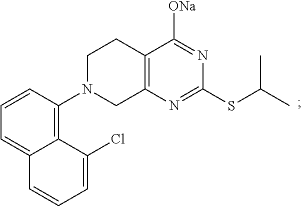

and
f′) reacting the final compound of step (e′) with an oxidizing agent, a base and/or an alkoxide, a polar solvent and, optionally, a catalyst to produce a final compound of step (f) with the following structure:

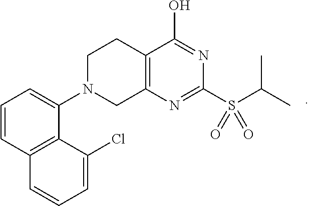 [SMILES: CC(C)S(=O)(=O)c1nc(O)c2c(n1)CN(c1cccc3cccc(Cl)c13)CC2]

In one embodiment, step (d′) is carried out at a temperature from about 20° C. to about 120° C.

In one embodiment, in step (d′), the polar solvent is selected from the group consisting of DMAc, DMF, 1,4-dioxane, THF, 2-MeTHF, MeCN, DMSO, NMP, and an alcohol with a formula R—OH, wherein R is alkyl, allyl or aryl.

In one embodiment, in step (d′), the alkoxide is selected from the group consisting of methoxide, ethoxide, iso-propoxide, tert-butoxide and tert-amylate, or ammonium or alkali salts thereof.

In one embodiment, step (e′) is carried out at a temperature from about 20° C. to about 120° C.

In one embodiment, in step (e′), the polar solvent is selected from the group consisting of DMAc, DMF, 1,4-dioxane, THF, 2-MeTHF, MeCN, DMSO, NMP, and an alcohol with a formula R—OH, wherein R is alkyl, allyl or aryl.

In one embodiment, in step (e′), the alkylating agent is selected from the group consisting of alkyl halides R—X (where R is methyl, ethyl, isopropyl, or benzyl and X is Cl, Br, I, alkyl sulfonate, aryl sulfonate, triflate or nonaflate), di-alkyl sulfate and carbonate.

In one embodiment, in step (e′), the alkoxide is selected from the group consisting of methoxide, ethoxide, iso-propoxide, tert-butoxide, tert-amylate, and alkali salts thereof.

In one embodiment, in step (e′), the inorganic base is selected from the group consisting of carbonate, bicarbonate, phosphate, hydroxide, and alkali salts thereof.

In one embodiment, step (f) is carried out at about −15° C. to about 60° C.

In one embodiment, in step (f), the oxidizing agent is selected from the group consisting of peracid, oxone, bleach, hydrogen peroxide, NaIO₄, perborate, percarbonate and urea hydrogen peroxide.

In one embodiment, in step (f′), the oxidizing agent is hydrogen peroxide.

In one embodiment, the catalyst is selected from the group consisting of sodium tungstate, phenylphosphonic acid, and methyltrioctylammonium hydrogensulfate.

In one embodiment, in step (f), the alkoxide is selected from the group consisting of methoxide, ethoxide, iso-propoxide, tert-butoxide and tert-amylate, or ammonium or alkali salts thereof.

In one embodiment, in step (f), the base is an inorganic base.

In one embodiment, the inorganic base is selected from the group consisting of carbonate, bicarbonate, phosphate, and alkali salts thereof.

In one embodiment, in step (f), the polar solvent is selected from the group consisting of acetonitrile and ROH, wherein R is methyl, ethyl, or 2-propyl.

In yet another embodiment, the steps (d) and (e) described above may be replaced by the following steps (d′″) and (e′″) to provide an alternative route to synthesize the final compound of step (e).
d′″) reacting the final compound of step (c) with an S-alkylated isothiourea salt, a solvent and a base to produce a final compound of step (d′″) with the following structure:

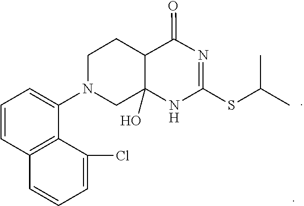 [SMILES: CC(C)SC1=NC(=O)C2CCN(c3cccc4cccc(Cl)c34)CC2(O)N1]

In one embodiment, step (d′″) is carried out at a temperature from about −20° C. to about 50° C.

In one embodiment, an alkyl group in the S-alkylated isothiourea salt is selected from the group consisting of methyl, ethyl, n-propyl, 2-propyl, cyclopropyl, n-butyl, i-butyl, s-butyl, t-butyl, cyclobutyl, n-pentyl, i-pentyl, s-pentyl, t-pentyl, cyclopentyl, n-hexyl, i-hexyl, s-hexyl, t-hexyl, cyclohexyl, and benzyl, and further wherein a counterion is selected from the group consisting of Cl⁻, Br⁻, I⁻, MsO⁻, TsO⁻, TfO⁻, BF₄⁻, SbF₆⁻, CF₃COO⁻, NO₃⁻· and SO₄²⁻.

In one embodiment, the solvent is an alcoholic solvent.

In one embodiment, the solvent is selected from the group consisting of DMAc, DMF, 1,4-dioxane, THF, 2-MeTHF, MeCN, DMSO, DCM, EtOAc, IPAc, and NMP.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the method further comprises step (e′″):
e′″) reacting the final compound of step (d′″) with a water miscible solvent and a base to produce a final compound of step (e′″) with the following structure:

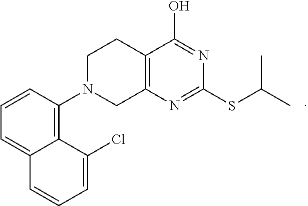 [SMILES: CC(C)Sc1nc(O)c2c(n1)CN(c1cccc3cccc(Cl)c13)CC2]

In one embodiment, step (e′″) is carried out at a temperature from about 0° C. to about 25° C.

In one embodiment, the water miscible solvent is selected from the group consisting of 2-propanol, tert-butanol, and acetonitrile.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In yet another embodiment, steps (e′) and (f) can be replaced by the following steps (e″) and (f′″) to provide an alternative route of synthesizing the compound of step (g) as follows.
(e″) reacting the final compound of step (d′) with a phosgene or a phosgene derivative, a polar aprotic solvent and optionally, a mineral acid to produce a final compound of step (e″) with the following structure:

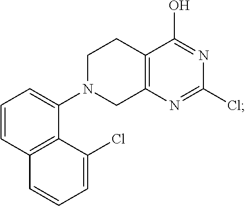 [SMILES: Oc1nc(Cl)nc2c1CCN(c1cccc3cccc(Cl)c13)C2]

and
(f″) reacting the final compound of step (e″) with an alkoxide and a polar aprotic solvent to produce a final compound of step (f″) with the following structure:

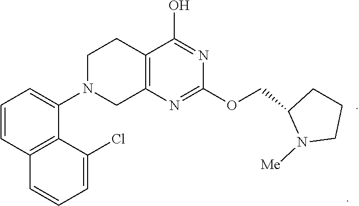 [SMILES: CN1CCC[C@H]1COc1nc(O)c2c(n1)CN(c1cccc3cccc(Cl)c13)CC2]

In one embodiment, step (e″) is carried out at a temperature from about 0° C. to about 120° C.

In one embodiment, in step (e″), the phosgene derivative is selected from the group consisting of diphosgene, triphosgene, thiophosgene and 1,1′-carbonyldiimidazole.

In one embodiment, in step (e″), the polar aprotic solvent is selected from the group consisting of DMAc, DMF, 1,4-dioxane, THF, 2-MeTHF, MeCN, DMSO, 2-propanol and NMP.

In one embodiment, in step (e″), the mineral acid is selected from the group consisting of hydrogen chloride, hydrochloric acid, sulfuric acid, nitric acid and phosphoric acid.

In one embodiment, step (f″) is carried out at a temperature from about 0° C. to about 120° C.

In one embodiment, in step (f″), the alkoxide is selected from the group consisting of iso-propoxide, tert-butoxide, tert-amylate, and alkali salts thereof.

In one embodiment, in step (f″), the polar aprotic solvent is selected from the group consisting of DMAc, DMF, 1,4-dioxane, THF, 2-MeTHF, MeCN, DMSO, 2-propanol and NMP.

In yet another embodiment, steps (h) and (i) can be replaced by the following: steps (h′) and (i′); steps (h″) and (i″); or steps (h′″) and (i′″) to provide alternative routes of synthesizing the compound of step (i) as follows:
h′) reacting the final compound of step (g) with a triflating agent, an acid, a polar aprotic solvent, an additive, and optionally a base, to produce a final compound of step (h′) with the following structure:

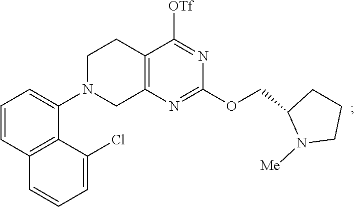 [SMILES: CN1CCC[C@H]1COc1nc2c(c(OS(=O)(=O)C(F)(F)F)n1)CCN(c1cccc3cccc(Cl)c13)C2]

and
i′) reacting the final compound of step (h′) with a salt of

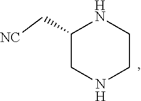

a base, and a polar aprotic solvent to produce a final compound of step (i′) with the following structure:

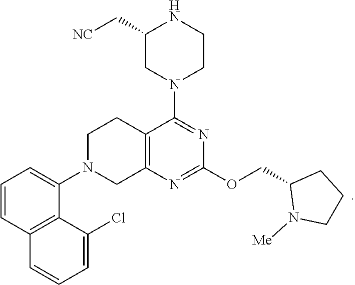 [SMILES: CN1CCC[C@H]1COc1nc2c(c(N3CCN[C@@H](CC#N)C3)n1)CCN(c1cccc3cccc(Cl)c13)C2]

In one embodiment, step (h′) is carried out at a temperature from about −80° C. to about 50° C.

In one embodiment, in step (h′), the triflating agent is selected from the group consisting of Tf₂O, CF₃SO₂Cl, and CF₃SO₂Br.

In one embodiment, in step (h′), the acid is selected from the group consisting of trifluoroacetic acid, triflic acid, and HCl.

In one embodiment, in step (h′), the polar aprotic solvent is selected from the group consisting of DMAc, DMF, 1,4-dioxane, THF, 2-MeTHF, MeCN, DMSO, 2-propanol and NMP.

In one embodiment, in step (h′), the base is an organic base.

In one embodiment, in step (h′), the organic base is selected from the group consisting of DIPEA, Et₃N, DABCO, and DBU.

In one embodiment, in step (h′), the base is an inorganic base.

In one embodiment, in step (h′), the inorganic base is selected from the group consisting of carbonate, bicarbonate, phosphate, and alkali salts thereof.

In one embodiment, in step (h′), the additive is selected from the group consisting of pyridine and a substituted pyridine, such as N,N-dimethylaminopyridine or lutidine.

In one embodiment, step (i′) is carried out at a temperature from about 0° C. to about 100° C.

In one embodiment, in step (i′), the salt is selected from the group consisting of HCl, TFA and HBr.

In one embodiment, in step (i′), the base is an organic base.

In one embodiment, in step (i′), the organic base is selected from the group consisting of DIPEA, Et₃N, DABCO, and DBU.

In one embodiment, in step (i′), the base is an inorganic base.

In one embodiment, in step (i′), the inorganic base is selected from the group consisting of carbonate, bicarbonate, phosphate, and alkali salts thereof.

In one embodiment, in step (i′), the polar aprotic solvent is selected from the group consisting of DMAc, DMF, 1,4-dioxane, THF, 2-MeTHF, MeCN, DMSO, 2-propanol and NMP.
h″) reacting the final compound of step (g) with a triflating agent, an acid, a base, and a polar aprotic solvent to produce a final compound of step (h″) with the following structure:

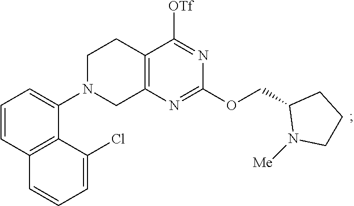 [SMILES: CN1CCC[C@H]1COc1nc2c(c(OS(=O)(=O)C(F)(F)F)n1)CCN(c1cccc3cccc(Cl)c13)C2]

and
i″) reacting the final compound of step (h″) with a salt of

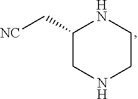

a base and a polar aprotic solvent to produce a final compound of step (i″) with the following structure:

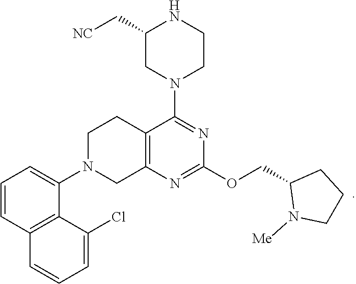 [SMILES: CN1CCC[C@H]1COc1nc2c(c(N3CCN[C@@H](CC#N)C3)n1)CCN(c1cccc3cccc(Cl)c13)C2]

In one embodiment, step (h″) is carried out at a temperature from about −80° C. to about 50° C.

In one embodiment, in step (h″), the triflating agent is selected from the group consisting of 2-[N,N-Bis(trifluoromethanesulfonyl)amino]-5-chloropyridine, 1-(Trifluoromethanesulfonyl)imidazole, 1-(Trifluoromethanesulfonyl)-1H-benzotriazole, N-(2-Pyridyl)bis(trifluoromethanesulfonimide), and N-Phenylbis(trifluoromethanesulfonimide).

In one embodiment, in step (h″), the acid is selected from the group consisting of trifluoroacetic acid, triflic acid, and HCl.

In one embodiment, in step (h″), the polar aprotic solvent is selected from the group consisting of DMAc, DMF, 1,4-dioxane, THF, 2-MeTHF, MeCN, DMSO, 2-propanol and NMP.

In one embodiment, in step (h″), the base is an organic base.

In one embodiment, in step (h″), the organic base is selected from the group consisting of DIPEA, Et₃N, DABCO, and DBU.

In one embodiment, in step (h″), the base is an inorganic base.

In one embodiment, in step (h″), the inorganic base is selected from the group consisting of carbonate, bicarbonate, phosphate, and alkali salts thereof.

In one embodiment, in step (h″), the additive is selected from the group consisting of pyridine and a substituted pyridine, such as N,N-dimethylaminopyridine or lutidine.

In one embodiment, step (i″) is carried out at a temperature from about 0° C. to about 100° C.

In one embodiment, in step (i″), the salt is selected from the group consisting of HCl, TFA and HBr.

In one embodiment, in step (i″), the base is an organic base.

In one embodiment, in step (i″), the organic base is selected from the group consisting of DIPEA, Et₃N, DABCO, and DBU.

In one embodiment, in step (i″), the base is an inorganic base.

In one embodiment, in step (i″), the inorganic base is selected from the group consisting of carbonate, bicarbonate, phosphate, and alkali salts thereof.

In one embodiment, in step (i″), the polar aprotic solvent is selected from the group consisting of DMAc, DMF, 1,4-dioxane, THF, 2-MeTHF, MeCN, DMSO, 2-propanol and NMP.
h′″) reacting the final compound of step (g) with an aryl- or heteroaryl sulfonyl, a base, a polar aprotic solvent, and an additive to produce a final compound of step (h′″) with the following structure.

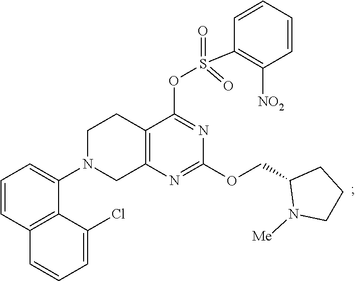 [SMILES: CN1CCC[C@H]1COc1nc2c(c(OS(=O)(=O)c3ccccc3[N+](=O)[O-])n1)CCN(c1cccc3cccc(Cl)c13)C2]

and
i′″) reacting the final compound of step (h′″) with a salt of

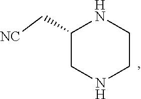

a base and a polar aprotic solvent to produce a final compound of step (i′″) with the following structure:

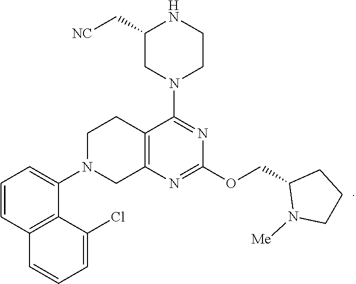 [SMILES: CN1CCC[C@H]1COc1nc2c(c(N3CCN[C@@H](CC#N)C3)n1)CCN(c1cccc3cccc(Cl)c13)C2]

In one embodiment, step (h′″) is carried out at a temperature from about −30° C. to about 100° C.

In one embodiment, in step (h′″), the aryl- or heteroaryl sulfonyl is ArSO₂X, wherein Ar is a substituted aromatic or heteroaromatic group and X is selected from the group consisting of F, Cl, Br, OMs, and OTs.

In one embodiment, Ar is selected from the group consisting of tolyl, mesityl, and nosyl.

In one embodiment, in step (h′″), the base is an organic base.

In one embodiment, in step (h′″), the organic base is selected from the group consisting of DIPEA, Et₃N, DABCO, and DBU.

In one embodiment, in step (h′″), the base is an inorganic base.

In one embodiment, in step (h′″), the inorganic base is selected from the group consisting of carbonate, bicarbonate, phosphate, and alkali salts thereof.

In one embodiment, in step (h′″), the polar aprotic solvent is selected from the group consisting of DMAc, DMF, 1,4-dioxane, THF, 2-MeTHF, MeCN, DMSO, 2-propanol and NMP.

In one embodiment, in step (h′″), the additive is selected from the group consisting of pyridine and a substituted pyridine.

In one embodiment, step (i′″) is carried out at a temperature from about 0° C. to about 100° C.

In one embodiment, in step (i′″), the salt is selected from the group consisting of HCl, TFA and HBr.

In one embodiment, in step (i′″), the base is an organic base.

In one embodiment, in step (i′″), the organic base is selected from the group consisting of DIPEA, Et₃N, DABCO, and DBU.

In one embodiment, in step (i′″), the base is an inorganic base.

In one embodiment, in step (i′″), the inorganic base is selected from the group consisting of carbonate, bicarbonate, phosphate, and alkali salts thereof.

In one embodiment, in step (i′″), the polar aprotic solvent is selected from the group consisting of DMAc, DMF, 1,4-dioxane, THF, 2-MeTHF, MeCN, DMSO, 2-propanol and NMP.

In one embodiment, the invention provides a method of synthesizing adagrasib, comprising the step of reacting

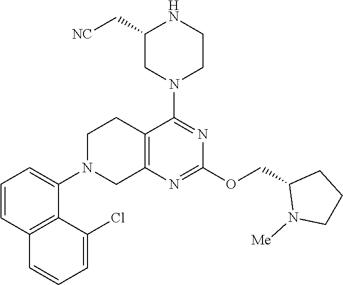 [SMILES: CN1CCC[C@H]1COc1nc2c(c(N3CCN[C@@H](CC#N)C3)n1)CCN(c1cccc3cccc(Cl)c13)C2]

with a salt of 2-fluoroacrylic acid, a base and a solvent to produce adagrasib.

In one embodiment, the invention provides a method of synthesizing adagrasib comprising:
a first reaction, wherein

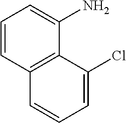 [SMILES: Nc1cccc2cccc(Cl)c12]

is reacted in a vessel with a 4-halobutyrate, a non-polar solvent, an iodide, and a base;
a second reaction, wherein a 2-halo-N-methoxy-N-methylacetamide, an aprotic solvent and a base are added to the vessel; and
a third reaction, wherein a base and an aprotic solvent are added to the vessel to produce a compound with the following structure:

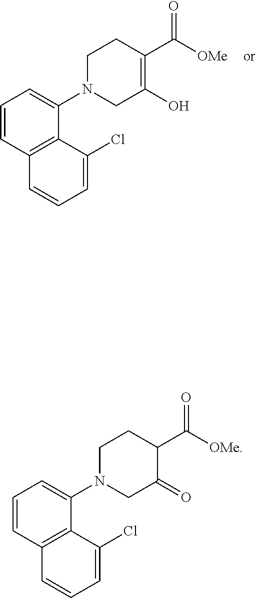 [SMILES: COC(=O)C1=C(O)CN(c2cccc3cccc(Cl)c23)CC1 ; COC(=O)C1CCN(c2cccc3cccc(Cl)c23)CC1=O]

In one embodiment, the invention provides a method of synthesizing adagrasib, comprising:
a first reaction, wherein

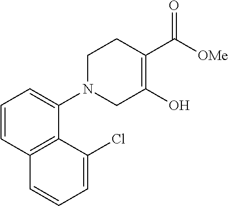 [SMILES: COC(=O)C1=C(O)CN(c2cccc3cccc(Cl)c23)CC1]

is reacted in a vessel with an S-alkylated isothiourea salt, a solvent and a base; and
a second reaction, wherein an acid, a triflating/mesylating agent and an aprotic solvent are added to the vessel to produce a compound with the following structure:

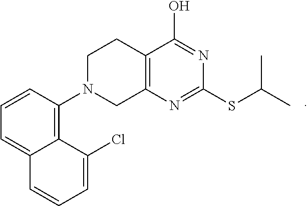 [SMILES: CC(C)Sc1nc(O)c2c(n1)CN(c1cccc3cccc(Cl)c13)CC2]

In one embodiment, the invention provides a method of synthesizing adagrasib, comprising:
a first reaction, wherein

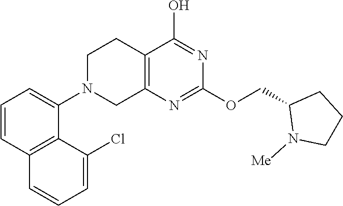 [SMILES: CN1CCC[C@H]1COc1nc(O)c2c(n1)CN(c1cccc3cccc(Cl)c13)CC2]

is reacted in a vessel with an activating agent, a base, a polar aprotic solvent, and an additive; and
a second reaction, wherein a salt of

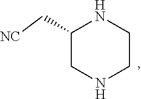

a base and a polar aprotic solvent are added to the vessel to produce a compound with the following structure:

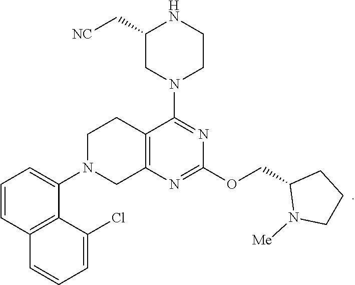 [SMILES: CN1CCC[C@H]1COc1nc2c(c(N3CCN[C@@H](CC#N)C3)n1)CCN(c1cccc3cccc(Cl)c13)C2]

In one embodiment, the invention provides a method of synthesizing adagrasib, comprising:
a first reaction, wherein

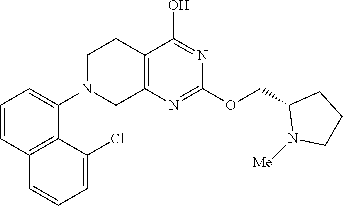 [SMILES: CN1CCC[C@H]1COc1nc(O)c2c(n1)CN(c1cccc3cccc(Cl)c13)CC2]

is reacted in a vessel with a triflating agent, an acid, a polar aprotic solvent, an additive, and optionally a base; and
a second reaction, wherein a salt of

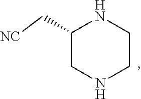

a base, and a polar aprotic solvent are added to the vessel to produce a compound with the following structure:

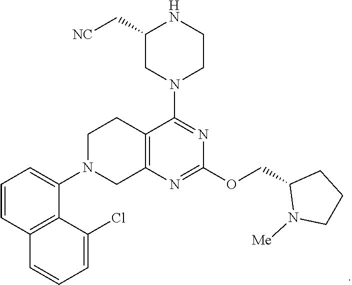 [SMILES: CN1CCC[C@H]1COc1nc2c(c(N3CCN[C@@H](CC#N)C3)n1)CCN(c1cccc3cccc(Cl)c13)C2]

In one embodiment, the invention provides a method of synthesizing adagrasib, comprising:
a first reaction, wherein

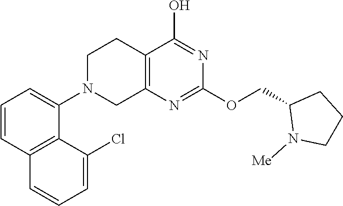 [SMILES: CN1CCC[C@H]1COc1nc(O)c2c(n1)CN(c1cccc3cccc(Cl)c13)CC2]

is reacted in a vessel with an aryl- or heteroaryl sulfonyl, a base, a polar aprotic solvent, and an additive, and
a second reaction, wherein a salt of

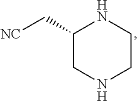

a base, and a polar aprotic solvent are added to the vessel to produce a compound with the following structure:

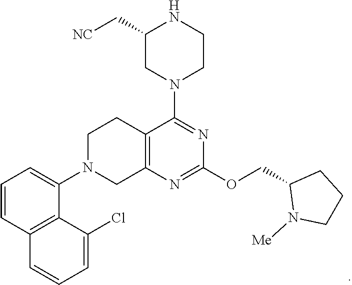 [SMILES: CN1CCC[C@H]1COc1nc2c(c(N3CCN[C@@H](CC#N)C3)n1)CCN(c1cccc3cccc(Cl)c13)C2]

The invention also encompasses each of the above steps by themselves (i.e., an embodiment that is directed to step (j); an embodiment that is directed to step (i), an embodiment that is directed to step (h), etc.), as well to combinations of the steps (i.e., an embodiment that is directed to step (i) and step (j); an embodiment that is directed to step (h), step (i), and step (j), etc).

Furthermore, it may not be necessary to isolate and/or purify final compounds of any of the steps described above. For example, it is possible to arrive at the compound of step (c) without isolating and/or purifying final compounds of step (a) or (b).

The invention also provides novel compounds of the following structures:

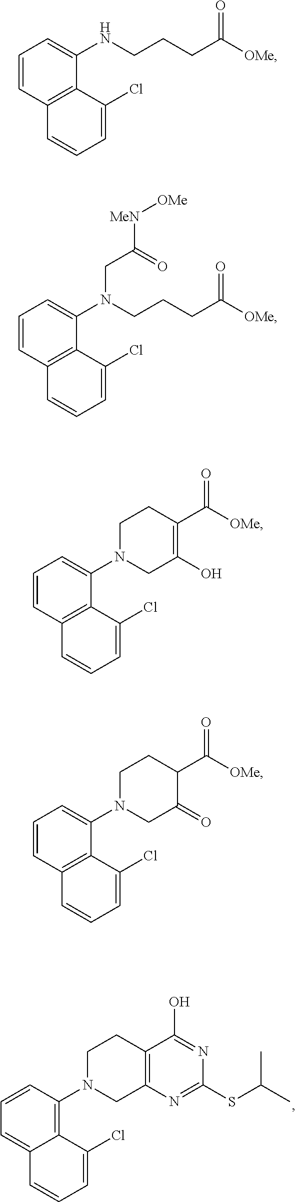 [SMILES: COC(=O)CCCNc1cccc2cccc(Cl)c12 ; COC(=O)CCCN(CC(=O)N(C)OC)c1cccc2cccc(Cl)c12 ; COC(=O)C1=C(O)CN(c2cccc3cccc(Cl)c23)CC1 ; COC(=O)C1CCN(c2cccc3cccc(Cl)c23)CC1=O ; CC(C)Sc1nc(O)c2c(n1)CN(c1cccc3cccc(Cl)c13)CC2]

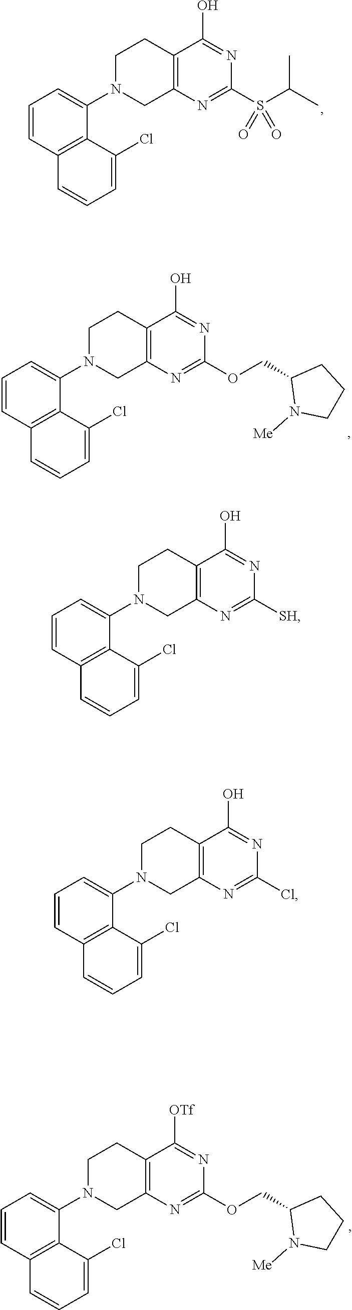 [SMILES: CC(C)S(=O)(=O)c1nc(O)c2c(n1)CN(c1cccc3cccc(Cl)c13)CC2 ; CN1CCC[C@H]1COc1nc(O)c2c(n1)CN(c1cccc3cccc(Cl)c13)CC2 ; Oc1nc(S)nc2c1CCN(c1cccc3cccc(Cl)c13)C2 ; Oc1nc(Cl)nc2c1CCN(c1cccc3cccc(Cl)c13)C2 ; CN1CCC[C@H]1COc1nc2c(c(OS(=O)(=O)C(F)(F)F)n1)CCN(c1cccc3cccc(Cl)c13)C2]

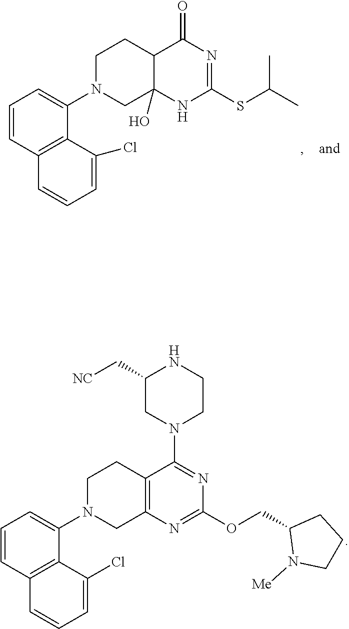 [SMILES: CC(C)SC1=NC(=O)C2CCN(c3cccc4cccc(Cl)c34)CC2(O)N1 ; CN1CCC[C@H]1COc1nc2c(c(N3CCN[C@@H](CC#N)C3)n1)CCN(c1cccc3cccc(Cl)c13)C2]

### DETAILED DESCRIPTION OF THE INVENTION

The present invention relates to new synthetic routes for synthesizing adagrasib, as well as to novel intermediates used in the provided routes.

Although there is a known method of synthesizing adagrasib (see WO 2019/099524), the synthesis provided by the present invention is much improved, in that it provides a higher isolated yield and a higher or similar purity overall.

The new and improved synthesis of MRTX849—adagrasib—features five high yielding steps with introduction of expensive building blocks at late-stage of the process.

The previous synthesis of adagrasib involved the introduction of the 2 expensive chiral pieces back-to-back in the first and second step. Using the new approach these two pieces are introduced toward the end of the synthesis, hence greatly improving the cost effectiveness of the production.

The new route also avoids the use of protecting steps—both Boc and Cbz protecting groups were eliminated—saving time and resources on their introduction and removal, making the route eco-friendlier.

The new route circumvents the major cost contributor, palladium catalysts. Increasingly more expensive, palladium was used in 2 out of 6 steps in the previous synthesis, dramatically driving cost up. The new procedure disclosed is completely transition metal-free.

### Definitions

Unless defined otherwise, all technical and scientific terms used herein have the same meaning as is commonly understood by one of skill in the art to which this invention belongs. All patents, patent applications, and publications referred to herein are incorporated by reference.

As used herein, “KRas G12C” refers to a mutant form of a mammalian KRas protein that contains an amino acid substitution of a cysteine for a glycine at amino acid position 12. The assignment of amino acid codon and residue positions for human KRas is based on the amino acid sequence identified by UniProtKB/Swiss-Prot P01116: Variant p.Gly12Cys.

A “KRas G12C-associated disease or disorder” as used herein refers to diseases or disorders associated with or mediated by or having a KRas G12C mutation. A non-limiting example of a KRas G12C-associated disease or disorder is a KRas G12C-associated cancer.

As used herein, the term “adagrasib” refers to the compound which has the name: 2-[(2S)-4-[7-(8-chloro-1-naphthyl)-2-[[(2S)-1-methylpyrrolidin-2-yl]methoxy]-6,8-dihydro-5H-pyrido[3,4-d]pyrimidin-4-yl]-1-(2-fluoroprop-2-enoyl)piperazin-2-yl]acetonitrile (also known as MRTX849) and which has the following structure:

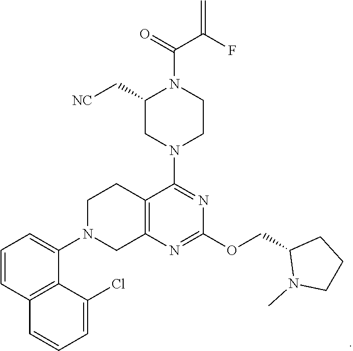 [SMILES: C=C(F)C(=O)N1CCN(c2nc(OC[C@@H]3CCCN3C)nc3c2CCN(c2cccc4cccc(Cl)c24)C3)C[C@@H]1CC#N]

Adagrasib is described, for example, in Example 478 of PCT Application WO 2019/099524.

The term “adagrasib” encompasses all chiral (enantiomeric and diastereomeric) and racemic forms of the compound.

In one embodiment, the term “adagrasib” includes salts of the above compound, for instance salts formed with inorganic acids such as hydrochloric acid, hydrobromic acid, sulfuric acid, phosphoric acid, nitric acid, salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid, and salts formed from quaternary ammoniums of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, cinnamoate, mandeloate, benzyloate, and diphenylacetate).

Whenever the application refers to a chemical compound, unless specifically stated otherwise, the compound encompasses all chiral (enantiomeric and diastereomeric) and racemic forms of the compound, as well as its tautomers, and any mixtures thereof.

“LG” refers to a leaving group and has the meaning conventionally associated with the term “leaving group” in synthetic organic chemistry; that is, an atom or group that is displaceable under alkylating or nucleophilic aromatic substitution conditions. The term “leaving group” includes, but is not limited to, halogen, for example chlorine and bromide; alkanesulfonyloxys, for example methanesulfonyloxy and ethanesulfonyloxy; arenesulfonyloxys, for example benzylsulfonyloxy and tosyloxy; thienyloxy; dihalophosphinoyloxy; tetrahalophosphaoxy; perfluoroalkanesulfonyloxys, for example trifluoromethanesulfonyloxy and the like. The leaving group should be selected so as to be chemically less reactive (except of course when the leaving group is bromine wherein it will be equally reactive) than the reacting group, bromine, to ensure proper reaction.

Unless the application specifies differently, “R” refers to a group such as alkyl, alkenyl, alkynyl, alkylene, alkenylene, alkynylene, carbocycle, cycloalkyl, heteroalkyl, heterocycle, aryl, aralkyl, or arylalkyl.

The term “alkyl” is intended to mean a straight chain or branched aliphatic group having from 1 to 12 carbon atoms, alternatively 1-8 carbon atoms, and alternatively 1-6 carbon atoms. Other examples of alkyl groups have from 2 to 12 carbon atoms, alternatively 2-8 carbon atoms and alternatively 2-6 carbon atoms. Examples of alkyl groups include, without limitation, methyl, ethyl, propyl, isopropyl, butyl, isobutyl, sec-butyl, tert-butyl, pentyl, hexyl and the like. A “C0” alkyl (as in “C0-C3alkyl”) is a covalent bond.

The term “alkenyl” is intended to mean an unsaturated straight chain or branched aliphatic group with one or more carbon-carbon double bonds, having from 2 to 12 carbon atoms, alternatively 2-8 carbon atoms, and alternatively 2-6 carbon atoms. Examples of alkenyl groups include, without limitation, ethenyl, propenyl, butenyl, pentenyl, and hexenyl.

The term “alkynyl” is intended to mean an unsaturated straight chain or branched aliphatic group with one or more carbon-carbon triple bonds, having from 2 to 12 carbon atoms, alternatively 2-8 carbon atoms, and alternatively 2-6 carbon atoms. Examples of alkynyl groups include, without limitation, ethynyl, propynyl, butynyl, pentynyl, and hexynyl.

The terms “alkylene,” “alkenylene,” or “alkynylene” as used herein are intended to mean an alkyl, alkenyl, or alkynyl group, respectively, as defined hereinabove, that is positioned between and serves to connect two other chemical groups. Examples of alkylene groups include, without limitation, methylene, ethylene, propylene, and butylene. Examples of alkenylene groups include, without limitation, ethenylene, propenylene, and butenylene. Examples of alkynylene groups include, without limitation, ethynylene, propynylene, and butynylene.

The term “carbocycle” as employed herein is intended to mean a cycloalkyl or aryl moiety.

The term “cycloalkyl” is intended to mean a saturated or unsaturated mono-, bi-, tri- or poly-cyclic hydrocarbon group having about 3 to 15 carbons, alternatively having 3 to 12 carbons, alternatively 3 to 8 carbons, alternatively 3 to 6 carbons, and alternatively 5 or 6 carbons. In certain embodiments, the cycloalkyl group is fused to an aryl, heteroaryl or heterocyclic group. Examples of cycloalkyl groups include, without limitation, cyclopenten-2-enone, cyclopenten-2-enol, cyclohex-2-enone, cyclohex-2-enol, cyclopropyl, cyclobutyl, cyclobutenyl, cyclopentyl, cyclopentenyl, cyclohexyl, cyclohexenyl, cycloheptyl, cyclooctyl, etc.

The term “heteroalkyl” is intended to mean a saturated or unsaturated, straight chain or branched aliphatic group, wherein one or more carbon atoms in the group are independently replaced by a heteroatom selected from the group consisting of O, S, and N.

The term “aryl” is intended to mean a mono-, bi-, tri- or polycyclic aromatic moiety, for example a C6-C14aromatic moiety, for example comprising one to three aromatic rings. Alternatively, the aryl group is a C6-C10aryl group, alternatively a C6aryl group. Examples of aryl groups include, without limitation, phenyl, naphthyl, anthracenyl, and fluorenyl.

The terms “aralkyl” or “arylalkyl” are intended to mean a group comprising an aryl group covalently linked to an alkyl group. If an aralkyl group is described as “optionally substituted”, it is intended that either or both of the aryl and alkyl moieties may independently be optionally substituted or unsubstituted. Alternatively, the aralkyl group is (C1-C6)alk(C6-C10)aryl, including, without limitation, benzyl, phenethyl, and naphthylmethyl. For simplicity, when written as “arylalkyl” this term, and terms related thereto, is intended to indicate the order of groups in a compound as “aryl-alkyl”. Similarly, “alkyl-aryl” is intended to indicate the order of the groups in a compound as “alkyl-aryl”.

As used herein, the term “pharmaceutically acceptable salt” refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

As used herein, the term “mineral acid” (or “inorganic acid”) refers to any acid derived from an inorganic compound that dissociates to produce hydrogen ions (H+) in water. Nonlimiting examples of mineral acids include hydrogen halides of the general formula HX (where X is F, Cl, Br or I), nitric acid, phosphoric acid, sulfuric acid, boric acid and perchloric acid.

As used herein, the term “organic acid” refers to any organic compound with acidic properties. Nonlimiting examples of organic acids include sulfonic acids of the general formula RSO₃H (where R can be alkyl, alkenyl, alkynyl, carbocycle, heterocycle, aryl and are define above), carboxylic acids (with one or several carboxylic acid sites) of the general formula RCO₂H (where R can be alkyl, alkenyl, alkynyl, carbocycle, heterocycle, aryl and are define above). Nonlimiting examples of organic acids are lactic acid, acetic acid, formic acid, citric acid, oxalic acid, uric acid, malic acid, and tartaric acid.

### Synthetic Schemes

The present invention, in one embodiment, provides new and improved methods of making adagrasib.

In one embodiment, the invention provides a method of synthesizing adagrasib, comprising step (a):
a) reacting a compound of the following structure:

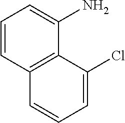 [SMILES: Nc1cccc2cccc(Cl)c12]

with a 4-halobutyrate, an aprotic solvent, an iodide, and a base to produce a final compound of step (a) with the following structure:

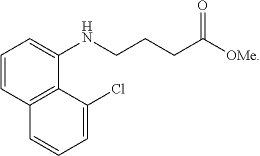 [SMILES: COC(=O)CCCNc1cccc2cccc(Cl)c12]

In one embodiment, step (a) is carried out at a temperature from about 20° C. to about 120° C.

In one embodiment, the 4-halobutyrate is 4-X(CH₂)₃CO₂R, wherein R is any alkyl or (hetero)aryl group selected from the group consisting of methyl, ethyl, propyl, and trifluoroethyl, and wherein X is any leaving group. In one embodiment, X is selected from the group consisting of Cl, Br, I, MsO, TsO, and TfO.

In one embodiment, the aprotic solvent is selected from the group consisting of toluene, anisole, xylene, dioxane, dichloromethane, dimethylformamide, dimethylacetamide, acetonitrile, 2-methyltetrahydrofuran, tetrahydrofuran, cyclopentyl methyl ether, methyl tert-butyl ether, and diethylene glycol dimethyl.

In one embodiment, the iodide is selected from the group consisting of sodium iodide, potassium iodide, and alkylated ammonium iodide.

In one embodiment, the base is an organic base.

In one embodiment, the organic base is selected from the group consisting of Diisopropylethylamine (DIPEA), triethylamine (Et₃N), triethylenediamine (DABCO), and 1,8-Diazabicyclo[5.4.0]undec-7-ene (DBU).

In another embodiment, the base is an inorganic base.

In one embodiment, the inorganic base is selected from the group consisting of carbonate, bicarbonate, and phosphate. An inorganic base can be used with any alkali such as lithium, sodium and potassium.

In one embodiment, the method further comprises step (b):
b) reacting the final compound of step (a) with a 2-halo-N-methoxy-N-methylacetamide, an aprotic solvent and a base to produce a final compound of step (b) with the following structure:

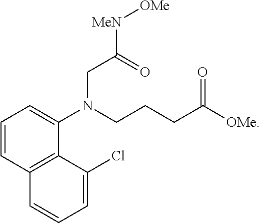 [SMILES: COC(=O)CCCN(CC(=O)N(C)OC)c1cccc2cccc(Cl)c12]

In one embodiment, step (b) is carried out at a temperature from about 20° C. to about 150° C.

In one embodiment, the 2-halo-N-methoxy-N-methylacetamide is XCH₂C(O)NMeOMe wherein X is selected from the group consisting of Cl, Br, I, MsO, TsO, and TfO.

In one embodiment, the aprotic solvent is selected from the group consisting of toluene, anisole, xylene, diethylene glycol dimethyl, DMAc, DMF, 1,4-dioxane, THF, 2-MeTHF, MeCN, DMSO, and NMP.

In one embodiment, step (b) further comprises an iodide.

In one embodiment, the iodide is selected from the group consisting of sodium iodide, potassium iodide, and alkylated ammonium iodide.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, and phosphate. An inorganic base can be used with any alkali such as lithium, sodium and potassium.

In one embodiment, the method further comprises step (c):
c) reacting the final compound of step (b) with a base and an aprotic solvent to produce a final compound of step (c) with the following structure:

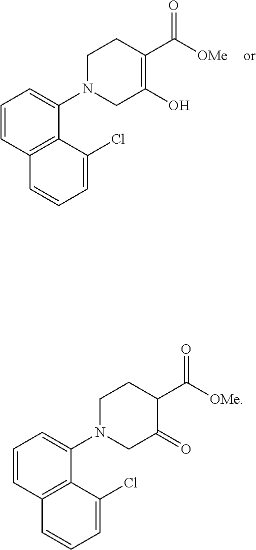 [SMILES: COC(=O)C1=C(O)CN(c2cccc3cccc(Cl)c23)CC1 ; COC(=O)C1CCN(c2cccc3cccc(Cl)c23)CC1=O]

In one embodiment, step (c) is carried out at a temperature from about −80° C. to about 25° C.

In one embodiment, the base is a bulky base. As used herein, the term “bulky base” means a sterically hindered base that is a poor nucleophile.

In one embodiment, the base is selected from the group consisting of lithium bis(trimethylsilyl)amide (LiHMDS), sodium bis(trimethylsilyl)amide (NaHMDS), potassium bis(trimethylsilyl)amide (KHMDS), lithium diisopropylamide (LDA), and lithium tetramethylpiperidide (LiTMP).

In one embodiment, the aprotic solvent is selected from the group consisting of toluene, anisole, xylene, diethylene glycol dimethyl, DMAc, DMF, 1,4-dioxane, THF, 2-MeTHF, MeCN, DMSO, and NMP.

In one embodiment, the method further comprises step (d):
d) reacting the final compound of step (c) with an S-alkylated isothiourea salt, a solvent and a base to produce a final compound of step (d) with the following structure:

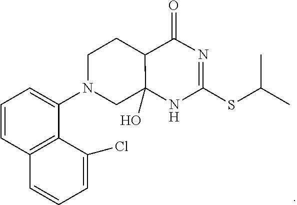 [SMILES: CC(C)SC1=NC(=O)C2CCN(c3cccc4cccc(Cl)c34)CC2(O)N1]

In one embodiment, step (d) is carried out at a temperature from about −20° C. to about 50° C.

In one embodiment, an alkyl group in the S-alkylated isothiourea salt is selected from the group consisting of methyl, ethyl, n-propyl, 2-propyl, cyclopropyl, n-butyl, i-butyl, s-butyl, t-butyl, cyclobutyl, n-pentyl, i-pentyl, s-pentyl, t-pentyl, cyclopentyl, n-hexyl, i-hexyl, s-hexyl, t-hexyl, cyclohexyl, and benzyl and further wherein a counterion is selected from the group consisting of Cl⁻, Br⁻, I⁻, MsO⁻, TsO⁻, TfO⁻, BF₄⁻, SbF₆⁻, CF₃COO⁻, NO₃⁻, and SO₄²⁻

In one embodiment, the solvent is an alcoholic solvent.

In one embodiment, the solvent is selected from the group consisting of DMAc, DMF, 1,4-dioxane, THF, 2-MeTHF, MeCN, DMSO, DCM, EtOAc, IPAc, and NMP.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the method further comprises step (e):
e) reacting the final compound of step (d) with an acid, a triflating agent and an aprotic solvent to produce a final compound of step (e) with the following structure:

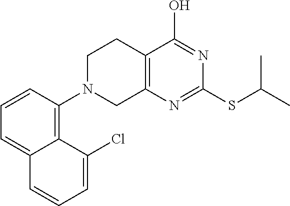 [SMILES: CC(C)Sc1nc(O)c2c(n1)CN(c1cccc3cccc(Cl)c13)CC2]

In one embodiment, step (e) is carried out at a temperature from about 0° C. to about 50° C.

In one embodiment, the acid is selected from the group consisting of trifluoroacetic acid, triflic acid, methanesulfonic acid, sulfuric acid and HCl.

In one embodiment, the triflating agent is selected from the group consisting of Tf₂O, CF₃SO₂Cl, and CF₃SO₂Br. The mesylating agent can comprise, but is not limited to, MsCl or (MeSO₂)₂O.

In one embodiment, the aprotic solvent is selected from the group consisting of toluene, anisole, xylene, diethylene glycol dimethyl, DMAc, DMF, 1,4-dioxane, THF, 2-MeTHF, MeCN, DMSO, and NMP.

In one embodiment, the method further comprises step (f):
f) reacting the final compound of step (e) with an oxidizing agent, a base and/or an alkoxide, a polar solvent and, optionally, a catalyst to produce a final compound of step (f) with the following structure:

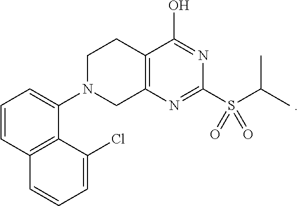 [SMILES: CC(C)S(=O)(=O)c1nc(O)c2c(n1)CN(c1cccc3cccc(Cl)c13)CC2]

In one embodiment, step (f) is carried out at about −15° C. to about 60° C.

In one embodiment, the oxidizing agent is selected from the group consisting of peracid, oxone, bleach, hydrogen peroxide, NaIO₄, perborate, percarbonate and urea hydrogen peroxide.

In one embodiment, the oxidizing agent is hydrogen peroxide.

In one embodiment, the catalyst is selected from the group consisting of sodium tungstate, phenylphosphonic acid, and methyltrioctylammonium hydrogensulfate.

In one embodiment, the alkoxide is selected from the group consisting of methoxide, ethoxide, iso-propoxide, tert-butoxide and tert-amylate, or ammonium or alkali salts thereof.

In one embodiment, the base is an inorganic base.

In one embodiment, the inorganic base is selected from the group consisting of carbonate, bicarbonate, phosphate, and alkali salts thereof.

In one embodiment, the polar solvent is selected from the group consisting of acetonitrile and ROH, wherein R is methyl, ethyl, or 2-propyl.

In one embodiment, the invention further comprises step (g):
g) reacting the final compound of step (f) with an alkoxide and a polar aprotic solvent to produce a final compound of step (g) with the following structure:

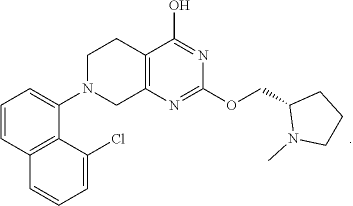 [SMILES: CN1CCC[C@H]1COc1nc(O)c2c(n1)CN(c1cccc3cccc(Cl)c13)CC2]

In one embodiment, step (g) is carried out at about −20° C. to about 100° C.

In one embodiment, the alkoxide is selected from the group consisting of iso-propoxide, tert-butoxide, tert-amylate, and alkali salts thereof.

In one embodiment, the polar aprotic solvent is selected from the group consisting of DMAc, DMF, 1,4-dioxane, THF, 2-MeTHF, MeCN, DMSO, 2-propanol and NMP.

In one embodiment, the invention further comprises step (h):
h) reacting the final compound of step (g) with an activating agent, a base, a polar aprotic solvent, and an additive, to produce a final compound of step (h) with the following structure:

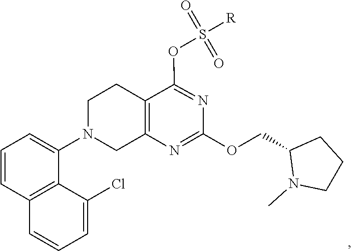

wherein R is selected from the group consisting of a substituted phenyl, methyl, ethyl, propyl, C₄F₉, and toluene.

In one embodiment, step (h) is carried out at a temperature from about −80° C. to about 120° C.

In one embodiment, the activating agent is selected from the group consisting of sulfonyl halide R—SO₂X (wherein R is tolyl, mesityl, nosyl, methyl, ethyl, or propyl and X is F, Cl, Br, Oms, or OTs), anhydride, and organic triflate reagent R¹—N-Tf₂ (where R¹ is phenyl, 5-chloro-2-pyridine, or 2-pyridine).

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the polar aprotic solvent is selected from the group consisting of DMAc, DMF, 1,4-dioxane, THF, 2-MeTHF, MeCN, DMSO, 2-propanol and NMP.

In one embodiment, the additive is selected from the group consisting of pyridine and a substituted pyridine.

In one embodiment, the invention further comprises step (i):
i) reacting the final compound of step (h) with a salt of

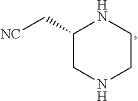

a base and a polar aprotic solvent to produce a final compound of step (i) with the following structure:

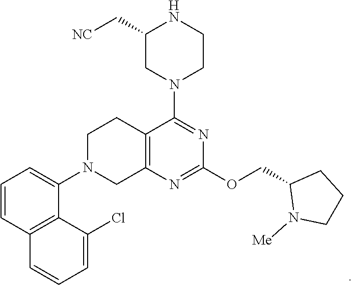 [SMILES: CN1CCC[C@H]1COc1nc2c(c(N3CCN[C@@H](CC#N)C3)n1)CCN(c1cccc3cccc(Cl)c13)C2]

In one embodiment, step (i) is carried out at a temperature from about 0° C. to about 100° C.

In one embodiment, the salt is selected from the group consisting of HCl, TFA and HBr.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the polar aprotic solvent is selected from the group consisting of DMAc, DMF, 1,4-dioxane, THF, 2-MeTHF, MeCN, DMSO, 2-propanol and NMP.

In one embodiment, the invention further comprises step (j):
j) reacting the final compound of step (i) with a salt of 2-fluoroacrylic acid, a solvent, and optionally a base to produce adagrasib.

In one embodiment, step (j) is carried out at a temperature from about −10° C. to about 50° C.

In one embodiment, the salt is a lithium, sodium, potassium, or ammonium salt.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the solvent is selected from the group consisting of DMAc, DMF, 1,4-dioxane, THE, 2-MeTHF, MeCN, DMSO, NMP, and an alcohol with a formula R—OH, wherein R is alkyl, allyl or aryl.

In one embodiment, the steps (d), (e), and (f) described above may be replaced by the following steps (d′), (e′) and (f) to provide an alternative route to synthesize the final compound of step (f).
d′) reacting the final compound of step (c) with an alkoxide and a polar solvent to produce a final compound of step (d′) with the following structure:

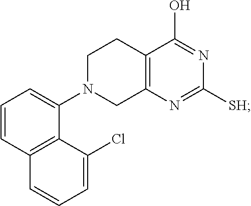 [SMILES: Oc1nc(S)nc2c1CCN(c1cccc3cccc(Cl)c13)C2]

e′) reacting the final compound of step (d′) with an alkylating agent, an inorganic base and/or an alkoxide, and a polar solvent to produce a final compound of step (e′) with the following structure:

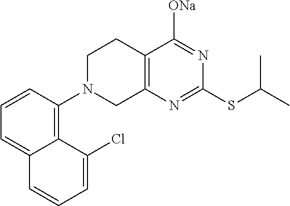

and
f′) reacting the final compound of step (e′) with an oxidizing agent, an alkoxide and/or an inorganic base, and a polar solvent to produce a final compound of step (f′) with the following structure:

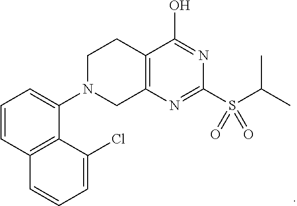 [SMILES: CC(C)S(=O)(=O)c1nc(O)c2c(n1)CN(c1cccc3cccc(Cl)c13)CC2]

In one embodiment, step (d′) is carried out at a temperature from about 20° C. to about 120° C.

In one embodiment, in step (d′), the polar solvent is selected from the group consisting of DMAc, DMF, 1,4-dioxane, THF, 2-MeTHF, MeCN, DMSO, NMP, and an alcohol with a formula R—OH, wherein R is alkyl, allyl or aryl.

In one embodiment, in step (d′), the alkoxide is selected from the group consisting of methoxide, ethoxide, iso-propoxide, tert-butoxide and tert-amylate, or ammonium or alkali salts thereof.

In one embodiment, step (e′) is carried out at a temperature from about 20° C. to about 120° C.

In one embodiment, in step (e′), the polar solvent is selected from the group consisting of DMAc, DMF, 1,4-dioxane, THF, 2-MeTHF, MeCN, DMSO, NMP, and an alcohol with a formula R—OH, wherein R is alkyl, allyl or aryl.

In one embodiment, in step (e′), the alkylating agent is selected from the group consisting of alkyl halides R—X (where R is methyl, ethyl, isopropyl, or benzyl and X is Cl, Br, I, alkyl sulfonate, aryl sulfonate, triflate or nonaflate), di-alkyl sulfate and carbonate.

In one embodiment, in step (e′), the alkoxide is selected from the group consisting of methoxide, ethoxide, iso-propoxide, tert-butoxide, tert-amylate, and alkali salts thereof.

In one embodiment, in step (e′), the inorganic base is selected from the group consisting of carbonate, bicarbonate, phosphate, hydroxide, and alkali salts thereof.

In one embodiment, step (f′) is carried out at about −15° C. to about 60° C.

In one embodiment, in step (f′), the oxidizing agent is selected from the group consisting of peracid, oxone, bleach, hydrogen peroxide, NaIO₄, perborate, percarbonate and urea hydrogen peroxide.

In one embodiment, in step (f′), the oxidizing agent is hydrogen peroxide.

In one embodiment, the catalyst is selected from the group consisting of sodium tungstate, phenylphosphonic acid, and methyltrioctylammonium hydrogensulfate.

In one embodiment, in step (f′), the alkoxide is selected from the group consisting of methoxide, ethoxide, iso-propoxide, tert-butoxide and tert-amylate, or ammonium or alkali salts thereof.

In one embodiment, in step (f′), the base is an inorganic base.

In one embodiment, the inorganic base is selected from the group consisting of carbonate, bicarbonate, phosphate, and alkali salts thereof.

In one embodiment, in step (f′), the polar solvent is selected from the group consisting of acetonitrile and ROH, wherein R is methyl, ethyl, or 2-propyl.

In yet another embodiment, the steps (d) and (e) described above may be replaced by the following steps (d′″) and (e′″) to provide an alternative route to synthesize the final compound of step (e).
d′″) reacting the final compound of step (c) with an S-alkylated isothiourea salt, a solvent and a base to produce a final compound of step (d′″) with the following structure:

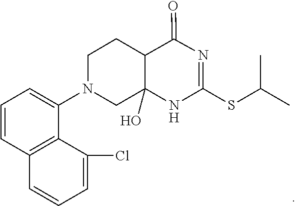 [SMILES: CC(C)SC1=NC(=O)C2CCN(c3cccc4cccc(Cl)c34)CC2(O)N1]

In one embodiment, step (d′″) is carried out at a temperature from about −20° C. to about 50° C.

In one embodiment, an alkyl group in the S-alkylated isothiourea salt is selected from the group consisting of methyl, ethyl, n-propyl, 2-propyl, cyclopropyl, n-butyl, i-butyl, s-butyl, t-butyl, cyclobutyl, n-pentyl, i-pentyl, s-pentyl, t-pentyl, cyclopentyl, n-hexyl, i-hexyl, s-hexyl, t-hexyl, cyclohexyl, and benzyl, and further wherein a counterion is selected from the group consisting of Cl⁻, Br⁻, I⁻, MsO⁻, TsO⁻, TfO⁻, BF₄⁻, SbF₆⁻, CF₃COO⁻, NO₃⁻, and SO₄²⁻.

In one embodiment, the solvent is an alcoholic solvent.

In one embodiment, the solvent is selected from the group consisting of DMAc, DMF, 1,4-dioxane, THF, 2-MeTHF, MeCN, DMSO, DCM, EtOAc, IPAc, and NMP.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In one embodiment, the method further comprises step (e′″):
e′″) reacting the final compound of step (d′″) with a water miscible solvent and a base to produce a final compound of step (e′″) with the following structure:

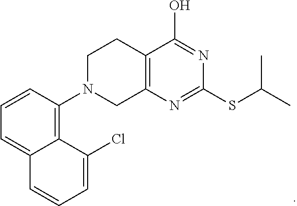 [SMILES: CC(C)Sc1nc(O)c2c(n1)CN(c1cccc3cccc(Cl)c13)CC2]

In one embodiment, step (e′″) is carried out at a temperature from about 0° C. to about 25° C.

In one embodiment, the water miscible solvent is selected from the group consisting of 2-propanol, tert-butanol, and acetonitrile.

In one embodiment, the base is an organic base.

In one embodiment, the organic base is selected from the group consisting of DIPEA, Et₃N, DABCO, and DBU.

In another embodiment, the base is an inorganic base.

In one embodiment, the inorganic base is selected from the group consisting of carbonate, bicarbonate, phosphate, and alkali salts thereof.

In yet another embodiment, steps (e′) and (f′) can be replaced by the following steps (e″) and (f′″) to provide an alternative route of synthesizing the compound of step (g) as follows.
(e″) reacting the final compound of step (d′) with a phosgene or a phosgene derivative, a polar aprotic solvent and a mineral acid to produce a final compound of step (e″) with the following structure:

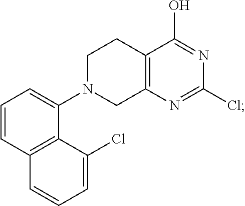 [SMILES: Oc1nc(Cl)nc2c1CCN(c1cccc3cccc(Cl)c13)C2]

and
(f″) reacting the final compound of step (e″) with an alkoxide and a polar aprotic solvent to produce a final compound of step (f″) with the following structure:

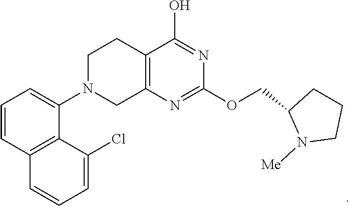 [SMILES: CN1CCC[C@H]1COc1nc(O)c2c(n1)CN(c1cccc3cccc(Cl)c13)CC2]

In one embodiment, step (e″) is carried out at a temperature from about 0° C. to about 120° C.

In one embodiment, in step (e″), the phosgene derivative is selected from the group consisting of diphosgene, triphosgene, thiophosgene and 1,1′-carbonyldiimidazole.

In one embodiment, in step (e″), the polar aprotic solvent is selected from the group consisting of DMAc, DMF, 1,4-dioxane, THF, 2-MeTHF, MeCN, DMSO, 2-propanol and NMP.

In one embodiment, in step (e″), the mineral acid is selected from the group consisting of hydrogen chloride, hydrochloric acid, sulfuric acid, nitric acid and phosphoric acid.

In one embodiment, step (f″) is carried out at a temperature from about 0° C. to about 120° C.

In one embodiment, in step (f″), the alkoxide is selected from the group consisting of iso-propoxide, tert-butoxide, tert-amylate, and alkali salts thereof.

In one embodiment, in step (f″), the polar aprotic solvent is selected from the group consisting of DMAc, DMF, 1,4-dioxane, THF, 2-MeTHF, MeCN, DMSO, 2-propanol and NMP.

In yet another embodiment, steps (h) and (i) can be replaced by the following: steps (h′) and (i′); steps (h″) and (i″); or steps (h′″) and (i′″) to provide alternative routes of synthesizing the compound of step (i) as follows.
h′) reacting the final compound of step (g) with a triflating agent, an acid, a polar aprotic solvent, an additive, and optionally a base, to produce a final compound of step (h′) with the following structure:

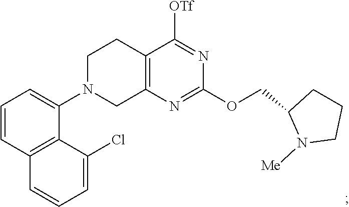 [SMILES: CN1CCC[C@H]1COc1nc2c(c(OS(=O)(=O)C(F)(F)F)n1)CCN(c1cccc3cccc(Cl)c13)C2]

and
i′) reacting the final compound of step (h′) with a salt of

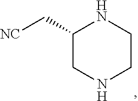

a base, and a polar aprotic solvent to produce a final compound of step (i′) with the following structure:

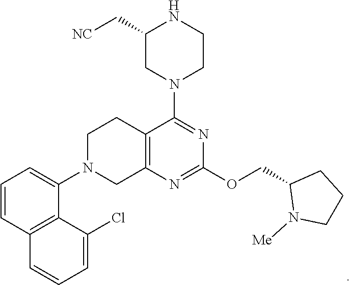 [SMILES: CN1CCC[C@H]1COc1nc2c(c(N3CCN[C@@H](CC#N)C3)n1)CCN(c1cccc3cccc(Cl)c13)C2]

In one embodiment, step (h′) is carried out at a temperature from about −80° C. to about 50° C.

In one embodiment, in step (h′), the triflating agent is selected from the group consisting of Tf₂O, CF₃SO₂Cl, and CF₃SO₂Br.

In one embodiment, in step (h′), the acid is selected from the group consisting of trifluoroacetic acid, triflic acid, and HCl.

In one embodiment, in step (h′), the polar aprotic solvent is selected from the group consisting of DMAc, DMF, 1,4-dioxane, THF, 2-MeTHF, MeCN, DMSO, 2-propanol and NMP.

In one embodiment, in step (h′), the base is an organic base.

In one embodiment, in step (h′), the organic base is selected from the group consisting of DIPEA, Et₃N, DABCO, and DBU.

In one embodiment, in step (h′), the base is an inorganic base.

In one embodiment, in step (h′), the inorganic base is selected from the group consisting of carbonate, bicarbonate, phosphate, and alkali salts thereof.

In one embodiment, in step (h′), the additive is selected from the group consisting of pyridine and a substituted pyridine, such as N,N-dimethylaminopyridine or lutidine.

In one embodiment, step (i′) is carried out at a temperature from about 0° C. to about 100° C.

In one embodiment, in step (i′), the salt is selected from the group consisting of HCl, TFA and HBr.

In one embodiment, in step (i′), the base is an organic base.

In one embodiment, in step (i′), the organic base is selected from the group consisting of DIPEA, Et₃N, DABCO, and DBU.

In one embodiment, in step (i′), the base is an inorganic base.

In one embodiment, in step (i′), the inorganic base is selected from the group consisting of carbonate, bicarbonate, phosphate, and alkali salts thereof.

In one embodiment, in step (i′), the polar aprotic solvent is selected from the group consisting of DMAc, DMF, 1,4-dioxane, THF, 2-MeTHF, MeCN, DMSO, 2-propanol and NMP.
h″) reacting the final compound of step (g) with a triflating agent, an acid, a polar aprotic solvent, an additive, and optionally a base, to produce a final compound of step (h″) with the following structure:

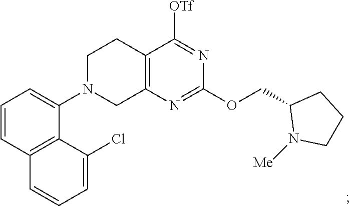 [SMILES: CN1CCC[C@H]1COc1nc2c(c(OS(=O)(=O)C(F)(F)F)n1)CCN(c1cccc3cccc(Cl)c13)C2]

i″) reacting the final compound of step (h″) with a salt of

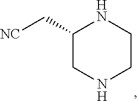

a base and a polar aprotic solvent to produce a final compound of step (i″) with the following structure:

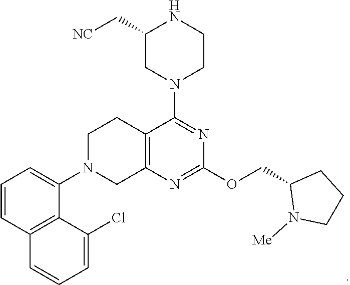 [SMILES: CN1CCC[C@H]1COc1nc2c(c(N3CCN[C@@H](CC#N)C3)n1)CCN(c1cccc3cccc(Cl)c13)C2]

In one embodiment, step (h″) is carried out at a temperature from about −80° C. to about 50° C.

In one embodiment, in step (h″), the triflating agent is selected from the group consisting of 2-[N,N-Bis(trifluoromethanesulfonyl)amino]-5-chloropyridine, 1-(Trifluoromethanesulfonyl)imidazole, 1-(Trifluoromethanesulfonyl)-1H-benzotriazole, N-(2-Pyridyl)bis(trifluoromethanesulfonimide), and N-Phenylbis(trifluoromethanesulfonimide).

In one embodiment, in step (h″), the acid is selected from the group consisting of trifluoroacetic acid, triflic acid, and HCl.

In one embodiment, in step (h″), the polar aprotic solvent is selected from the group consisting of DMAc, DMF, 1,4-dioxane, THF, 2-MeTHF, MeCN, DMSO, 2-propanol and NMP.

In one embodiment, in step (h″), the base is an organic base.

In one embodiment, in step (h″), the organic base is selected from the group consisting of DIPEA, Et₃N, DABCO, and DBU.

In one embodiment, in step (h″), the base is an inorganic base.

In one embodiment, in step (h″), the inorganic base is selected from the group consisting of carbonate, bicarbonate, phosphate, and alkali salts thereof.

In one embodiment, in step (h″), the additive is selected from the group consisting of pyridine and a substituted pyridine, such as N,N-dimethylaminopyridine or lutidine.

In one embodiment, step (i″) is carried out at a temperature from about 0° C. to about 100° C.

In one embodiment, in step (i″), the salt is selected from the group consisting of HCl, TFA and HBr.

In one embodiment, in step (i″), the base is an organic base.

In one embodiment, in step (i″), the organic base is selected from the group consisting of DIPEA, Et₃N, DABCO, and DBU.

In one embodiment, in step (i″), the base is an inorganic base.

In one embodiment, in step (i″), the inorganic base is selected from the group consisting of carbonate, bicarbonate, phosphate, and alkali salts thereof.

In one embodiment, in step (i″), the polar aprotic solvent is selected from the group consisting of DMAc, DMF, 1,4-dioxane, THF, 2-MeTHF, MeCN, DMSO, 2-propanol and NMP.
h′″) reacting the final compound of step (g) with an aryl- or heteroaryl sulfonyl, a base and a polar aprotic solvent to produce a final compound of step (h′″) with the following structure:

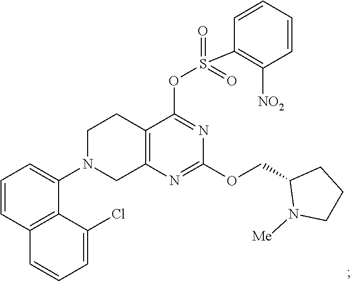 [SMILES: CN1CCC[C@H]1COc1nc2c(c(OS(=O)(=O)c3ccccc3[N+](=O)[O-])n1)CCN(c1cccc3cccc(Cl)c13)C2]

and
i′″) reacting the final compound of step (h′″) with a salt of

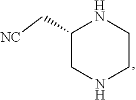

a base and a polar aprotic solvent to produce a final compound of step (i′″) with the following structure:

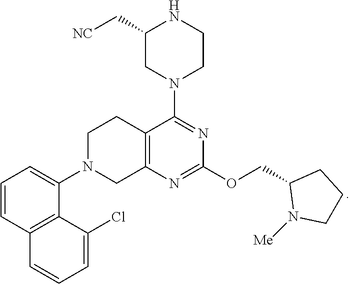 [SMILES: CN1CCC[C@H]1COc1nc2c(c(N3CCN[C@@H](CC#N)C3)n1)CCN(c1cccc3cccc(Cl)c13)C2]

In one embodiment, step (h′″) is carried out at a temperature from about −30° C. to about 100° C.

In one embodiment, in step (h′″), the aryl- or heteroaryl sulfonyl is ArSO₂X, wherein Ar is a substituted aromatic or heteroaromatic group and X is selected from the group consisting of F, Cl, Br, OMs, and OTs.

In one embodiment, Ar is selected from the group consisting of tolyl, mesityl, and nosyl.

In one embodiment, in step (h′″), the base is an organic base.

In one embodiment, in step (h′″), the organic base is selected from the group consisting of DIPEA, Et₃N, DABCO, and DBU.

In one embodiment, in step (h′″), the base is an inorganic base.

In one embodiment, in step (h′″), the inorganic base is selected from the group consisting of carbonate, bicarbonate, phosphate, and alkali salts thereof.

In one embodiment, in step (h′″), the polar aprotic solvent is selected from the group consisting of DMAc, DMF, 1,4-dioxane, THF, 2-MeTHF, MeCN, DMSO, 2-propanol and NMP.

In one embodiment, in step (h′″), the additive is selected from the group consisting of pyridine and a substituted pyridine.

In one embodiment, step (i′″) is carried out at a temperature from about 0° C. to about 100° C.

In one embodiment, in step (i′″), the salt is selected from the group consisting of HCl, TFA and HBr.

In one embodiment, in step (i′″), the base is an organic base.

In one embodiment, in step (i′″), the organic base is selected from the group consisting of DIPEA, Et₃N, DABCO, and DBU.

In one embodiment, in step (i′″), the base is an inorganic base.

In one embodiment, in step (i′″), the inorganic base is selected from the group consisting of carbonate, bicarbonate, phosphate, and alkali salts thereof.

In one embodiment, in step (i′″), the polar aprotic solvent is selected from the group consisting of DMAc, DMF, 1,4-dioxane, THF, 2-MeTHF, MeCN, DMSO, 2-propanol and NMP.

In one embodiment, the invention provides a method of synthesizing adagrasib, comprising the step of:
a) reacting a compound of the following structure:

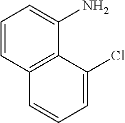 [SMILES: Nc1cccc2cccc(Cl)c12]

with a 4-halobutyrate, a non-polar solvent, an iodide, and a base to produce a final compound of step (a) with the following structure:

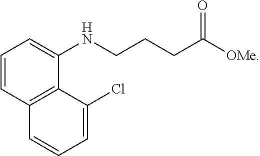 [SMILES: COC(=O)CCCNc1cccc2cccc(Cl)c12]

In one embodiment, step (a) is carried out at a temperature from about 20° C. to about 120° C.

In one embodiment, the method further comprises step (b):
b) reacting the final compound of step (a) with a 2-halo-N-methoxy-N-methylacetamide, an aprotic solvent and a base to produce a final compound of step (b) with the following structure:

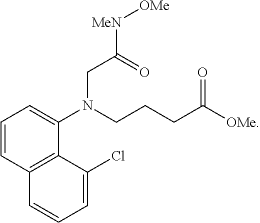 [SMILES: COC(=O)CCCN(CC(=O)N(C)OC)c1cccc2cccc(Cl)c12]

In one embodiment, step (b) is carried out at a temperature from about 20° C. to about 150° C.

In one embodiment, the method further comprises step (c):
c) reacting the final compound of step (b) with a base and an aprotic solvent to produce a final compound of step (c) with the following structure:

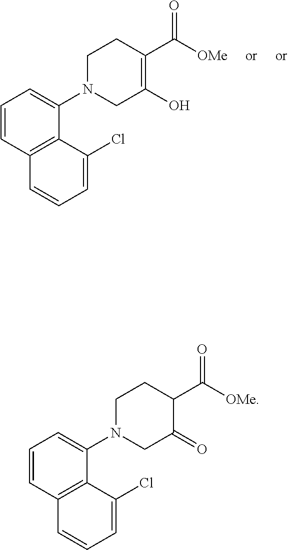 [SMILES: COC(=O)C1=C(O)CN(c2cccc3cccc(Cl)c23)CC1 ; COC(=O)C1CCN(c2cccc3cccc(Cl)c23)CC1=O]

In one embodiment, step (c) is carried out at a temperature from about −80° C. to about 25° C.

In one embodiment, the method further comprises step (d):
d) reacting the final compound of step (c) with an S-alkylated isothiourea salt, a solvent and a base to produce a final compound of step (d) with the following structure:

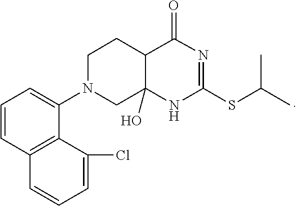 [SMILES: CC(C)SC1=NC(=O)C2CCN(c3cccc4cccc(Cl)c34)CC2(O)N1]

In one embodiment, step (d) is carried out at a temperature from about −20° C. to about 50° C.

In one embodiment, the method further comprises step (e):
e) reacting the final compound of step (d) with an acid, a triflating/mesylating agent, and an aprotic solvent to produce a final compound of step (e) with the following structure:

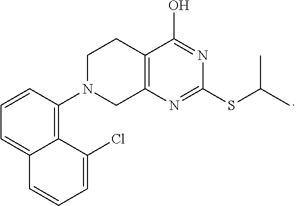 [SMILES: CC(C)Sc1nc(O)c2c(n1)CN(c1cccc3cccc(Cl)c13)CC2]

In one embodiment, step (e) is carried out at a temperature from about −20° C. to about 80° C.

In one embodiment, the method further comprises step (f):
f) reacting the final compound of step (e) with an oxidizing agent, a base and/or an alkoxide, a polar solvent, and, optionally, a catalyst to produce a final compound of step (f) with the following structure:

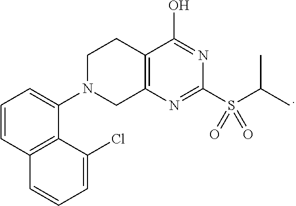 [SMILES: CC(C)S(=O)(=O)c1nc(O)c2c(n1)CN(c1cccc3cccc(Cl)c13)CC2]

In one embodiment, step (f) is carried out at about −15° C. to about 60° C.

In one embodiment, the invention further comprises step (g):
g) reacting the final compound of step (f) with an alkoxide and a polar aprotic solvent to produce a final compound of step (g) with the following structure:

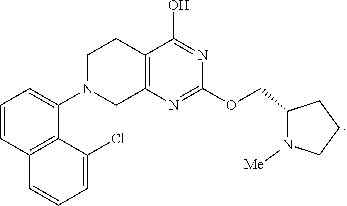 [SMILES: CN1CCC[C@H]1COc1nc(O)c2c(n1)CN(c1cccc3cccc(Cl)c13)CC2]

In one embodiment, the invention further comprises step (h):
h) reacting the final compound of step (g) with an activating agent, a base, a polar aprotic solvent and an additive, to produce a final compound of step (h) with the following structure:

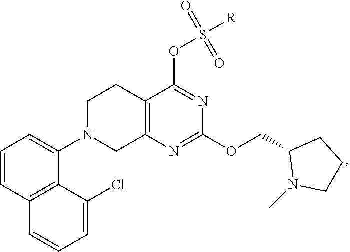

wherein R is selected from the group consisting of a substituted phenyl, methyl, ethyl, propyl, C₄F₉, and toluene.

In one embodiment, step (h) is carried out at a temperature from about −80° C. to about 120° C.

In one embodiment, the invention further comprises step (i):
i) reacting the final compound of step (h) with a salt of

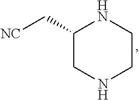

a base and a polar aprotic solvent to produce a final compound of step (i) with the following structure:

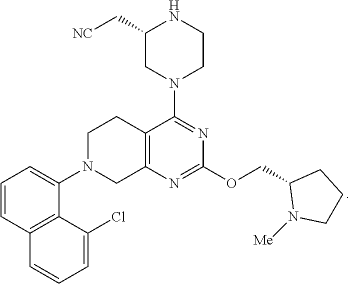 [SMILES: CN1CCC[C@H]1COc1nc2c(c(N3CCN[C@@H](CC#N)C3)n1)CCN(c1cccc3cccc(Cl)c13)C2]

In one embodiment, step (i) is carried out at a temperature from about 0° C. to about 100° C.

In one embodiment, the invention further comprises step (j):
j) reacting the final compound of step (i) with a salt of 2-fluoroacrylic acid, a solvent and optionally a base to produce adagrasib.

In one embodiment, step (j) is carried out at a temperature from about −10° C. to about 50° C.

In one embodiment, the steps (d), (e), and (f) described above may be replaced by the following steps (d′), (e′) and (f′) to provide an alternative route to synthesize the final compound of step (f).
d′) reacting the final compound of step (c) with an alkoxide and a polar solvent to produce a final compound of step (d′) with the following structure:

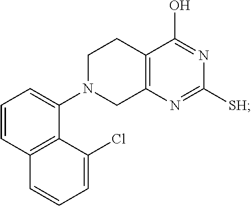 [SMILES: Oc1nc(S)nc2c1CCN(c1cccc3cccc(Cl)c13)C2]

and
e′) reacting the final compound of step (d′) with an alkylating agent, an inorganic base and/or an alkoxide, and a polar solvent to produce a final compound of step (e′) with the following structure:

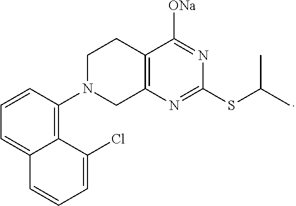

In one embodiment, step (d′) is carried out at a temperature from about 20° C. to about 120° C.

In one embodiment, step (e′) is carried out at a temperature from about 20° C. to about 120° C.
f′) reacting the final compound of step (e′) with an oxidizing agent, an alkoxide and/or an inorganic base, and a polar solvent to produce a final compound of step (f′) with the following structure:

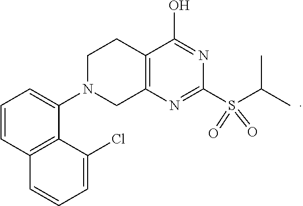 [SMILES: CC(C)S(=O)(=O)c1nc(O)c2c(n1)CN(c1cccc3cccc(Cl)c13)CC2]

In one embodiment, step (f′) is carried out at a temperature from about −15° C. to about 60° C.

In yet another embodiment, the steps (d) and (e) described above may be replaced by the following steps (d′″) and (e′″) to provide an alternative route to synthesize the final compound of step (e).
d′″) reacting the final compound of step (c) with an S-alkylated isothiourea salt, a solvent and a base to produce a final compound of step (d′″) with the following structure:

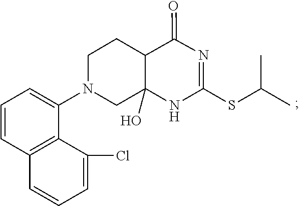 [SMILES: CC(C)SC1=NC(=O)C2CCN(c3cccc4cccc(Cl)c34)CC2(O)N1]

and
e′″) reacting the final compound of step (d′″) with a water miscible solvent and a base to produce a final compound of step (e′″) with the following structure:

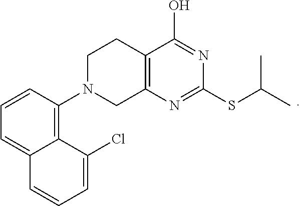 [SMILES: CC(C)Sc1nc(O)c2c(n1)CN(c1cccc3cccc(Cl)c13)CC2]

In one embodiment, step (e′″) is carried out at a temperature from about 0° C. to about 25° C.

In yet another embodiment, steps (e′) and (f′) can be replaced by the following steps (e″) and (f′″) to provide an alternative route of synthesizing the compound of step (g) as follows.
(e″) reacting the final compound of step (d′) with a phosgene or a phosgene derivative, a polar aprotic solvent and a mineral acid to produce a final compound of step (e″) with the following structure:

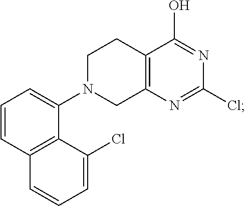 [SMILES: Oc1nc(Cl)nc2c1CCN(c1cccc3cccc(Cl)c13)C2]

and
(f″) reacting the final compound of step (e″) with an alkoxide and a polar aprotic solvent to produce a final compound of step (f″) with the following structure:

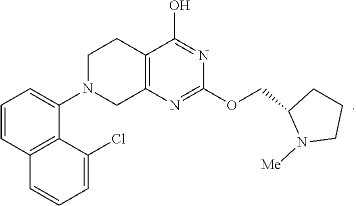 [SMILES: CN1CCC[C@H]1COc1nc(O)c2c(n1)CN(c1cccc3cccc(Cl)c13)CC2]

In one embodiment, step (e″) is carried out at a temperature from about 0° C. to about 120° C.

In one embodiment, step (f″) is carried out at a temperature from about 0° C. to about 120° C.

In yet another embodiment, steps (h) and (i) can be replaced by the following: steps (h′) and (i′); steps (h″) and (i″); or steps (h′″) and (i′″) to provide an alternative route of synthesizing the compound of step (i) as follows.
h′) reacting the final compound of step (g) with a triflating agent, an acid, a polar aprotic solvent, an additive, and optionally a base, to produce a final compound of step (h′) with the following structure:

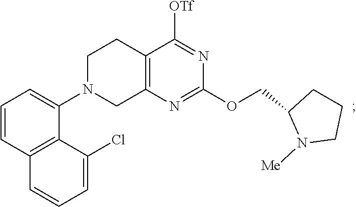 [SMILES: CN1CCC[C@H]1COc1nc2c(c(OS(=O)(=O)C(F)(F)F)n1)CCN(c1cccc3cccc(Cl)c13)C2]

and
i′) reacting the final compound of step (h′) with a salt of

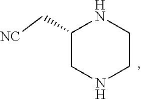

a base, and a polar aprotic solvent to produce a final compound of step (i′) with the following structure:

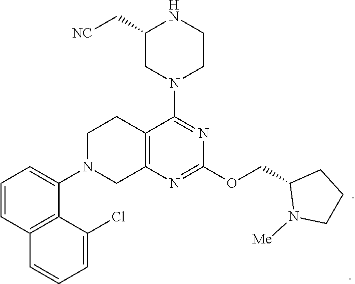 [SMILES: CN1CCC[C@H]1COc1nc2c(c(N3CCN[C@@H](CC#N)C3)n1)CCN(c1cccc3cccc(Cl)c13)C2]

In one embodiment, step (h′) is carried out at a temperature from about −80° C. to about 50° C.

In one embodiment, step (i′) is carried out at a temperature from about 0° C. to about 100° C.
h″) reacting the final compound of step (g) with a triflating agent, an acid, a base, and a polar aprotic solvent to produce a final compound of step (h″) with the following structure:

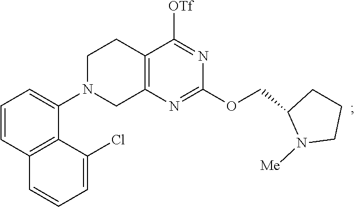 [SMILES: CN1CCC[C@H]1COc1nc2c(c(OS(=O)(=O)C(F)(F)F)n1)CCN(c1cccc3cccc(Cl)c13)C2]

and
i″) reacting the final compound of step (h″) with a salt of

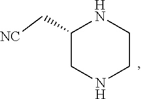

a base and a polar aprotic solvent to produce a final compound of step (i″) with the following structure:

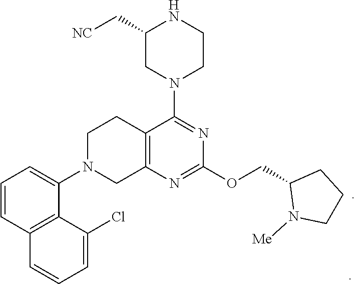 [SMILES: CN1CCC[C@H]1COc1nc2c(c(N3CCN[C@@H](CC#N)C3)n1)CCN(c1cccc3cccc(Cl)c13)C2]

In one embodiment, step (h″) is carried out at a temperature from about −80° C. to about 50° C.

In one embodiment, step (i″) is carried out at a temperature from about 0° C. to about 100° C.
h′″) reacting the final compound of step (g) with an aryl- or heteroaryl sulfonyl, a base and a polar aprotic solvent to produce a final compound of step (h′″) with the following structure:

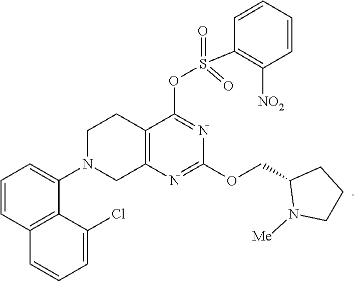 [SMILES: CN1CCC[C@H]1COc1nc2c(c(OS(=O)(=O)c3ccccc3[N+](=O)[O-])n1)CCN(c1cccc3cccc(Cl)c13)C2]

In one embodiment, step (h′″) is carried out at a temperature from about −30° C. to about 100° C.
i′″) reacting the final compound of step (h′″) with a salt of

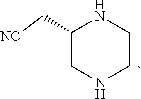

a base and a polar aprotic solvent to produce a final compound of step (i′″) with the following structure:

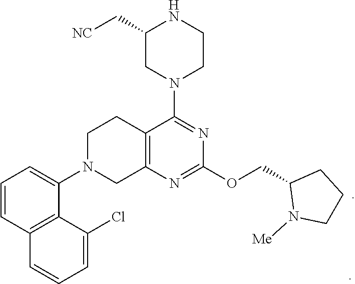 [SMILES: CN1CCC[C@H]1COc1nc2c(c(N3CCN[C@@H](CC#N)C3)n1)CCN(c1cccc3cccc(Cl)c13)C2]

In one embodiment, step (i′″) is carried out at a temperature from about 0° C. to about 100° C.

In one embodiment, the invention provides a method of synthesizing adagrasib, comprising the step of reacting

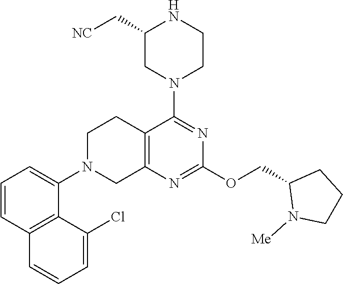 [SMILES: CN1CCC[C@H]1COc1nc2c(c(N3CCN[C@@H](CC#N)C3)n1)CCN(c1cccc3cccc(Cl)c13)C2]

with a salt of 2-fluoroacrylic acid, a base and a solvent to produce adagrasib.

The invention also encompasses each of the above steps by themselves (i.e., an embodiment that is directed to step (j); an embodiment that is directed to step (i); an embodiment that is directed to step (h), etc.), as well to combinations of the steps (i.e., an embodiment that is directed to step(i) and step (j); an embodiment that is directed to step (h), step (i), and step (j), etc).

In one embodiment, the invention provides a method of synthesizing adagrasib, comprising the steps of:
reacting

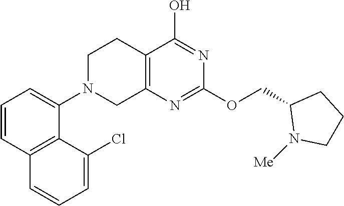 [SMILES: CN1CCC[C@H]1COc1nc(O)c2c(n1)CN(c1cccc3cccc(Cl)c13)CC2]

with an activating agent, a base, a polar aprotic solvent, and an additive, to produce:

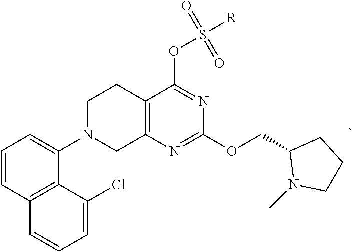

wherein R is selected from the group consisting of a substituted phenyl, methyl, ethyl, propyl, C₄F₉, and toluene;
reacting

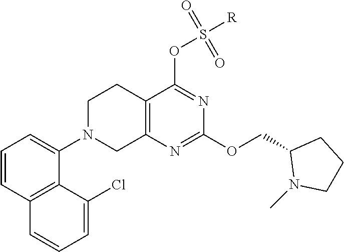

with a salt of

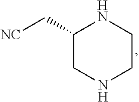

a base and a polar aprotic solvent to produce

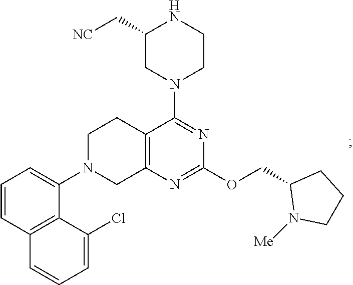 [SMILES: CN1CCC[C@H]1COc1nc2c(c(N3CCN[C@@H](CC#N)C3)n1)CCN(c1cccc3cccc(Cl)c13)C2]

reacting

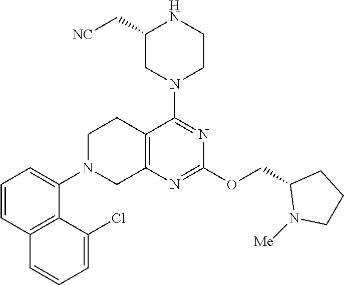 [SMILES: CN1CCC[C@H]1COc1nc2c(c(N3CCN[C@@H](CC#N)C3)n1)CCN(c1cccc3cccc(Cl)c13)C2]

with a salt of 2-fluoroacrylic acid, a base and a solvent to produce adagrasib.

In one embodiment, the invention provides a method of synthesizing adagrasib comprising the steps of:
reacting

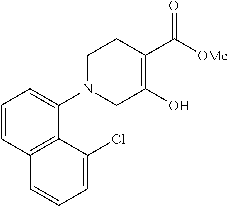 [SMILES: COC(=O)C1=C(O)CN(c2cccc3cccc(Cl)c23)CC1]

with an S-alkylated isothiourea salt, a solvent and a base to produce:

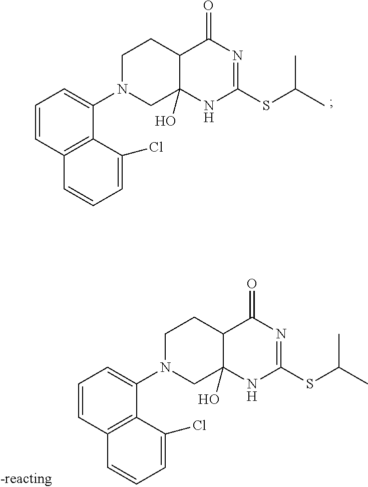 [SMILES: CC(C)SC1=NC(=O)C2CCN(c3cccc4cccc(Cl)c34)CC2(O)N1 ; CC(C)SC1=NC(=O)C2CCN(c3cccc4cccc(Cl)c34)CC2(O)N1]

with an acid, a triflating/mesylating agent, and an aprotic solvent to produce:

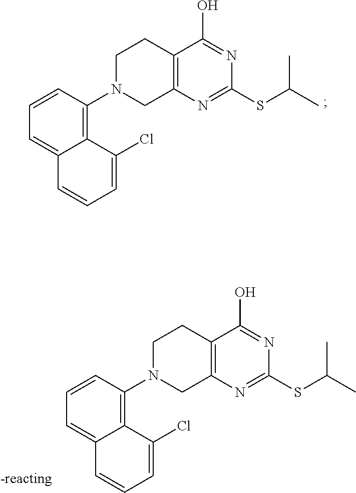 [SMILES: CC(C)Sc1nc(O)c2c(n1)CN(c1cccc3cccc(Cl)c13)CC2 ; CC(C)Sc1nc(O)c2c(n1)CN(c1cccc3cccc(Cl)c13)CC2]

with an oxidizing agent, a base and/or an alkoxide, a polar solvent and, optionally, a catalyst to produce:

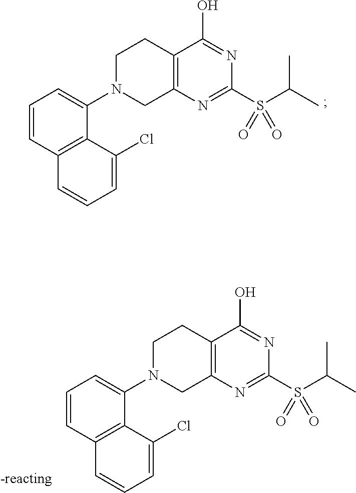 [SMILES: CC(C)S(=O)(=O)c1nc(O)c2c(n1)CN(c1cccc3cccc(Cl)c13)CC2 ; CC(C)S(=O)(=O)c1nc(O)c2c(n1)CN(c1cccc3cccc(Cl)c13)CC2]

with an alkoxide and a polar aprotic solvent to produce:

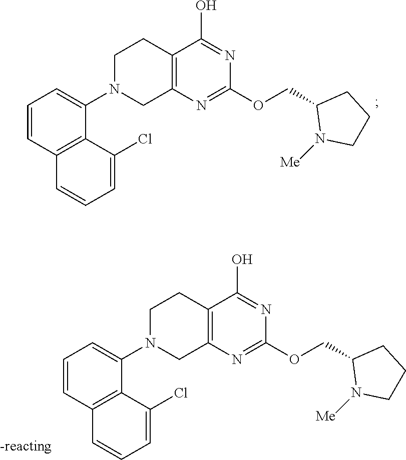 [SMILES: CN1CCC[C@H]1COc1nc(O)c2c(n1)CN(c1cccc3cccc(Cl)c13)CC2 ; CN1CCC[C@H]1COc1nc(O)c2c(n1)CN(c1cccc3cccc(Cl)c13)CC2]

with an activating agent, a base, a polar aprotic solvent, and an additive, to produce:

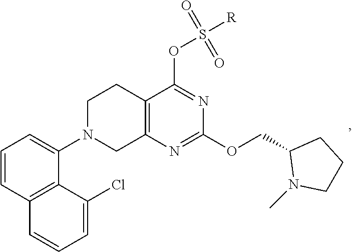

wherein R is selected from the group consisting of a substituted phenyl, methyl, ethyl, propyl, C₄F₉, and toluene;
reacting

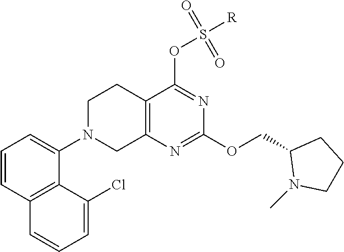

with a salt of

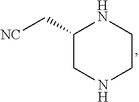

a base and a polar aprotic solvent to produce

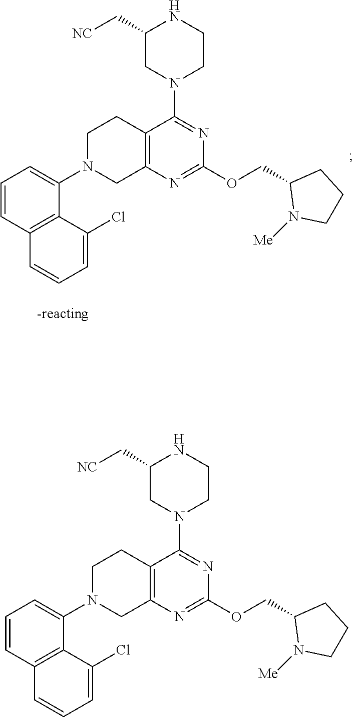 [SMILES: CN1CCC[C@H]1COc1nc2c(c(N3CCN[C@@H](CC#N)C3)n1)CCN(c1cccc3cccc(Cl)c13)C2 ; CN1CCC[C@H]1COc1nc2c(c(N3CCN[C@@H](CC#N)C3)n1)CCN(c1cccc3cccc(Cl)c13)C2]

with a salt of 2-fluoroacrylic acid, a base and a solvent to produce adagrasib.

In one embodiment, the invention provides a method of synthesizing adagrasib, comprising the steps of:
reacting

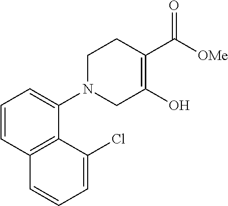 [SMILES: COC(=O)C1=C(O)CN(c2cccc3cccc(Cl)c23)CC1]

with an alkoxide and a polar solvent to produce:

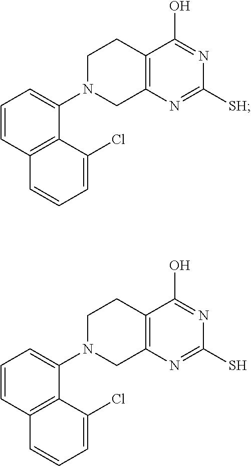 [SMILES: Oc1nc(S)nc2c1CCN(c1cccc3cccc(Cl)c13)C2 ; Oc1nc(S)nc2c1CCN(c1cccc3cccc(Cl)c13)C2]

with an alkylating agent, an inorganic base and/or an alkoxide, and a polar solvent to produce:

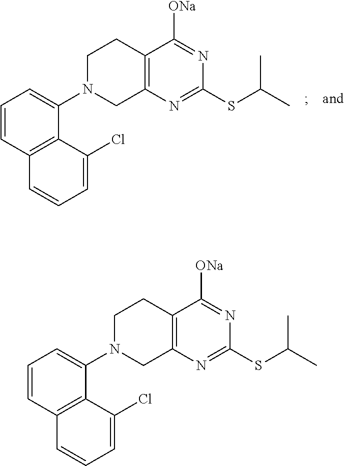

with an oxidizing agent, a base and/or an alkoxide, a polar solvent and, optionally, a catalyst to produce:

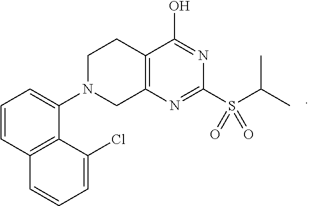 [SMILES: CC(C)S(=O)(=O)c1nc(O)c2c(n1)CN(c1cccc3cccc(Cl)c13)CC2]

The invention also provides novel compounds of the following structures:

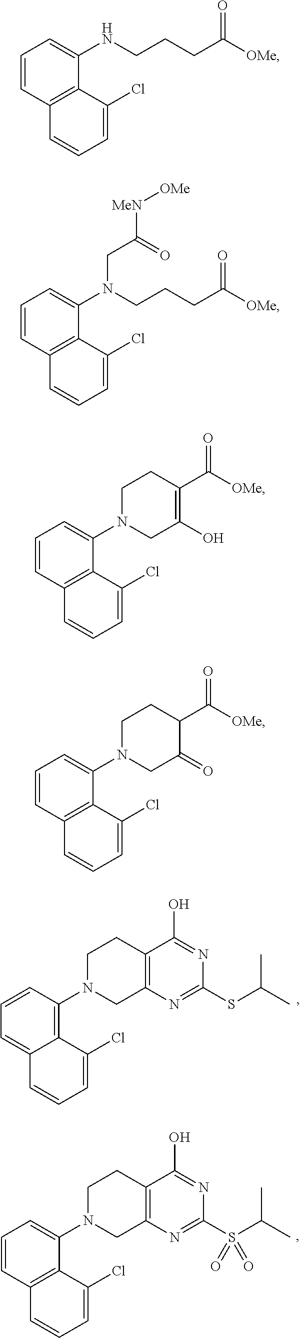 [SMILES: COC(=O)CCCNc1cccc2cccc(Cl)c12 ; COC(=O)CCCN(CC(=O)N(C)OC)c1cccc2cccc(Cl)c12 ; COC(=O)C1=C(O)CN(c2cccc3cccc(Cl)c23)CC1 ; COC(=O)C1CCN(c2cccc3cccc(Cl)c23)CC1=O ; CC(C)Sc1nc(O)c2c(n1)CN(c1cccc3cccc(Cl)c13)CC2 ; CC(C)S(=O)(=O)c1nc(O)c2c(n1)CN(c1cccc3cccc(Cl)c13)CC2]

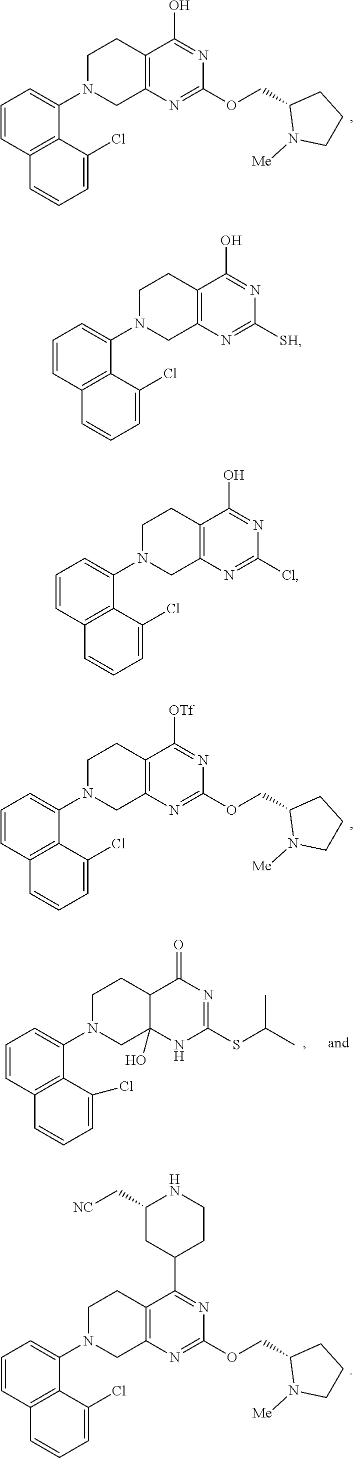 [SMILES: CN1CCC[C@H]1COc1nc(O)c2c(n1)CN(c1cccc3cccc(Cl)c13)CC2 ; Oc1nc(S)nc2c1CCN(c1cccc3cccc(Cl)c13)C2 ; Oc1nc(Cl)nc2c1CCN(c1cccc3cccc(Cl)c13)C2 ; CN1CCC[C@H]1COc1nc2c(c(OS(=O)(=O)C(F)(F)F)n1)CCN(c1cccc3cccc(Cl)c13)C2 ; CC(C)SC1=NC(=O)C2CCN(c3cccc4cccc(Cl)c34)CC2(O)N1 ; CN1CCC[C@H]1COc1nc2c(c(C3CCN[C@@H](CC#N)C3)n1)CCN(c1cccc3cccc(Cl)c13)C2]

The following Examples are intended to illustrate further certain embodiments of the invention and are not intended to limit the scope of the invention.

### Example 1

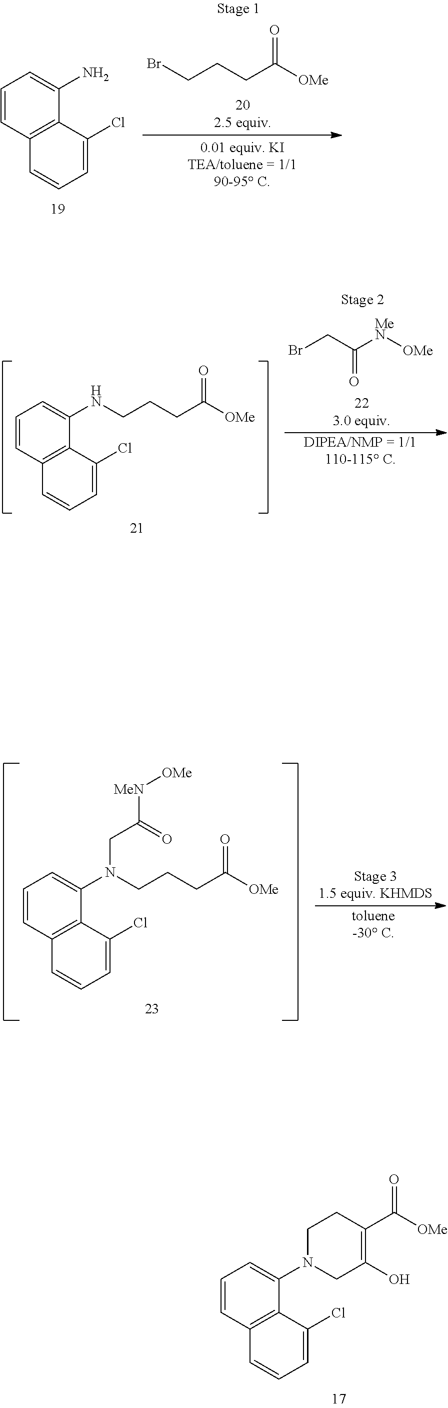

Stage 1: To a 100-L glass reactor was charged with amine 19 (2 kg), KI (20 g), PhMe (7 kg), methyl-4-bromobutyrate (5.68 kg), and TEA (5.8 kg) sequentially. The reaction was heated to 90° C. and stirred for 17 hrs. The reaction mixture was cooled to room temperature, followed by the addition of toluene (7 kg) and H₂O (8 kg). After stirring for 1 hour and then stopped to allow phase separation, the bottom aqueous layer was drained and the organic layer was concentrated to give crude 21.

Stage 2: To a 100-L reactor was charged with the stage 1 crude 21, NMP (5.2 kg), Weinreb fragment 22 (7.0 kg), DIPEA (4.4 kg). The mixture was heated to 100-110° C. and stirred for 19 h. The mixture was then cooled to room temperature and then added with 20% citric acid aqueous solution (8 kg). After stirring for 30 min at room temperature and then stopped to allow phase separation, the bottom aqueous layer was transferred to a 50-L reactor which was washed with toluene (7 kg). The result organic layer was then transferred back to the 100-L reactor. The combined layer was washed with 5% sodium bicarbonate aqueous solution (8.0 kg), 10% brine (8.0 kg) sequentially. The organic layer was then separated and concentrated to give crude 23.

Stage 3: To a 50-L reactor was charged with crude 23, toluene (3.4 kg). The mixture was cooled to −35° C. and KHMDS (15% w/w, 22.4 kg) in toluene was added in 3 hours, during which the temperature was maintained at −33 to −28° C. Another portion of KHMDS (15% w/w, 3.3 kg) in toluene was added and the result reaction was stirred for 14 hours at −33° C. Another 100-L reactor charged with a 20% citric acid aqueous solution (8 kg) was cooled to 0-5° C. and added with the reaction solution. The temperature of the stirred mixture was then adjusted to 25° C. and let the phase settle. The bottom aqueous layer was transferred out and further washed with toluene (3.4 kg). The two organic layers were combined and added with 20% citric acid (4.0 kg) aqueous solution and water (4.0 kg). The mixture was stirred for 30 minutes and then drained the bottom aqueous layer. The resulting organic layer was washed with 5% sodium bicarbonate (8.0 kg), 10% brine (8.0 kg) sequentially, and then polished by filtration through a bed of charcoal/celite. The filtrate was concentrated and added with isopropanol (3.15 kg)/heptane (5.84 kg). The result mixture was warmed to 60-65° C. to dissolve solids and then cooled down to 44° C. in 1 hour. 50 g ketoester 17 seeds and heptane (2.74 kg) were then added sequentially. Slowly cool down the mixture to −10° C. in 15 hours. Filtration and cake wash with heptane (2.75 kg) and isopropanol (0.40 kg) mixed solvent. The product was dried at 60° C. for 48 hours to afford 2.56 kg ketoester 17 as a light brown powder (60% overall yield in 3 steps).

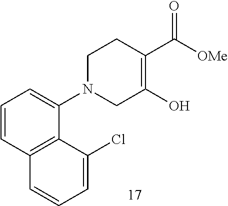 [SMILES: COC(=O)C1=C(O)CN(c2cccc3cccc(Cl)c23)CC1]

17: brown powder (2.56 kg, 89.2% assay) 60% yield. R_{f}=0.76 (heptane/ethyl acetate=1/1).

¹H NMR (400 MHz, CDCl₃) δ 12.03 (s, 1H), 7.75 (dd, J=8.1, 1.3 Hz, 1H), 7.61 (dd, J=8.2, 1.2 Hz, 1H), 7.55 (dd, J=7.5, 1.3 Hz, 1H), 7.44 (t, J=7.8 Hz, 1H), 7.34 (t, J=7.8 Hz, 1H), 7.22 (dd, J=7.6, 1.2 Hz, 1H), 3.92 (dd, J=17.4, 1.5 Hz, 1H), 3.84 (s, 3H), 3.52 (dt, J=17.4, 2.0 Hz, 1H), 3.44 (dddd, J=11.9, 5.1, 3.3, 1.4 Hz, 1H), 3.07 (ddd, J=11.9, 9.8, 4.1 Hz, 1H), 2.73 (dddt, J=13.6, 9.6, 5.6, 1.9 Hz, 1H), 2.41-2.31 (m, 1H). ¹³C NMR (100 MHz, CDCl₃) δ 172.5, 169.1, 148.2, 137.4, 130.1, 129.7, 128.2, 126.4, 126.0, 125.6, 125.0, 118.5, 96.4, 54.8, 51.6, 50.5, 22.4. HRMS: (ESI) m/z: [M+H]⁺ Calcd for C₁₇H₁₇O₃ClN 318.0892, Found: 318.0884.

### Example 2

The following pre-alkylated isothiourea were synthesized according to the reported literature procedures: 16a,¹ 16b,¹ 16c,² 16e,³ 16f,³ 16g⁴. 16d is commercially available.

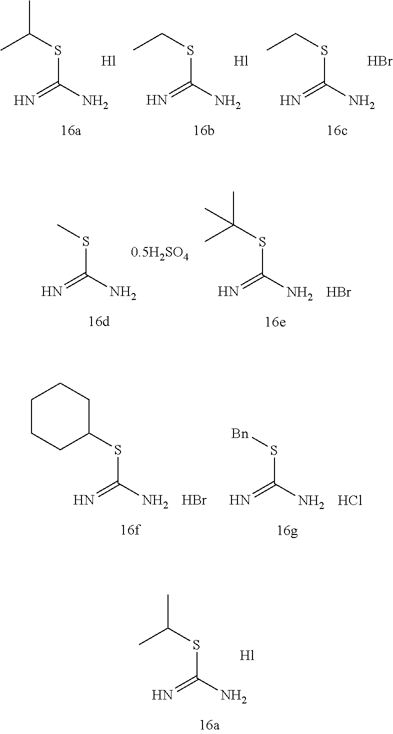

16a and 16b description is available at Sanmartin, C.; Dominguez, M. V.; Cordeu, L.; Cubedo, E.; Garcia-Foncillas, J.; Font, M.; Palop, J. A. Synthesis and Biological Evaluation of 2,4,6-Functionalized Derivatives of Pyrido[2,3-d]pyrimidines as Cytotoxic Agents and Apoptosis Inducers. Arch. Pharm. 2008, 341, 28-41.

16a was prepared as follows.

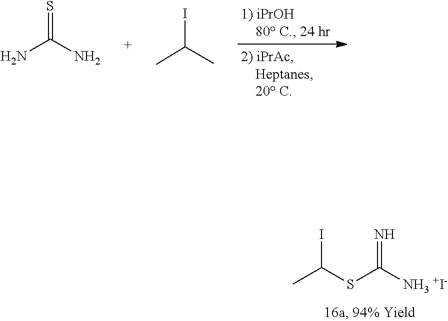

A clean 200 L glass-lined reactor was inerted with nitrogen. 75 L of isopropanol was charged and followed by the addition of 10.0 kg of thiourea (131 mol, 1 equiv.). Temperature was controlled to 20° C., and the mixture was stirred at 100 rpm. 24.6 kg of 2-iodopropane was charged (145 mol, 1.10 equiv.). 5 L of isopropanol was used to rinse the 2-iodopropane charging lines, and the reaction mixture was heated to a temperature of 75-78° C. over a period of 2 hr. Temperature was adjusted to 80° C. and the reaction mixture was stirred for 20 hr, at which point the mixture was sampled for HPLC analysis to ensure full conversion of starting material (Not more than [NMT]0.5% a/a thiourea to product). The in-process control (IPC) showed 0.34% a/a of thiourea and the reaction was progressed to the next stages. Temperature was adjusted to 85° C. and the reactor was placed under 0.04 MPa of reduced pressure to distill isopropanol. Distillation was stopped when the residual volume of liquid in the reactor reached a quantity of 40 L (4 v/w). 60 L of isopropyl acetate was loaded into the reactor, and it was then heated to 98° C. with 0.04 MPa of reduced pressure to distill the isopropyl acetate. Distillation was stopped when the remaining mixture reached a volume of 40 L (4 v/w). 30 L of isopropyl acetate was charged to the reaction mixture. It was again heated to 100° C. and evacuated by 0.04 MPa to distill the isopropyl acetate mixture to a level of 40 L (4 v/w). The mixture was sampled to check the water content (IPC: NMT 3%, actual value 0.02%) and isopropanol content (IPC: NMT 10%, actual value 7%). Contents of the reactor were cooled to 20-25° C., and isothiourea seed was charged (12.5 g, 0.125%, w/w). The mixture was stirred for 2 hr, and then 20 L of n-heptane was charged over a period of 2 hr. The slurry was stirred for an additional 2 hr, prior to draining the contents of the reactor into a Nutsche Filter for isolation. 13 L of isopropyl acetate and 13 L of n-heptane were charged to the reactor, mixed, and then used to rinse the filter cake. This process of rinsing the reactor and solids in the Nutsche filter was repeated three times, and then the filter cake was checked to ensure color was white to yellow (actual value, light yellow). The isolated solids were transferred to a drier with relative humidity under 40% and were dried for 12 hr at 25-30° C. with pressure reduced by 0.8 bar. Solids were checked to ensure water content was NMT 0.5% (actual value 0.19%). 30.56 kg (51.9 wt % assay) of light-yellow solids 16a was collected with 99.4% a/a purity and yield of 94.4%. Proton and carbon NMR match values reported in the literature. ¹H NMR (400 MHz, CD₃CN) δ 8.45 (s, 2H), 7.88 (s, 2H), 3.99 (dt, J=13.4, 6.7 Hz, 1H), 1.39 (d, J=6.8 Hz, 6H). ¹³C NMR (101 MHz, CD₃CN) δ 171.53 (s), 37.70 (s), 22.13 (s).

16c description is available at Brand, E.; Brand, F. C. Guanidoacetic Acid. Org. Synth. 1942, 22, 59.

16e and 16f description is available at Sprague, J. M.; Johnson, T. B. The Preparation of Alkyl Sulfonyl Chlorides from Isothioureas. II. J. Am. Chem. Soc. 1937, 59, 1837-1840.

16g description is available at Yang, Z.; Xu, J. Preparation of Alkanesulfonyl Chlorides from S-Alkyl Isothiourea Salts via N-Chlorosuccinimide Mediated Oxidative Chlorosulfonation. Org. Synth. 2014, 91, 116-124.

16a: yellow solid (89% yield). ¹HNMR (400 MHz, DMSO-d₆) δ=6 9.14-8.63 (m, 4H), 3.91 (dt, J=13.1, 6.5 Hz, 1H), 1.32 (d, J=6.5 Hz, 6H).

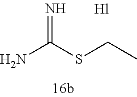

16b: orange solid (90% yield). ¹HNMR (400 MHz, DMSO-d₆) δ=8.92 (s, 4H), 3.5 (q, J=8.0 Hz, 2H), 1.25 (t, J=8.0 Hz, 3H).

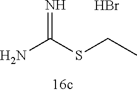

16c: off-white solid (89% yield). ¹HNMR (400 MHz, DMSO-d₆) δ=8.92 (s, 4H), 3.13-3.18 (m, 2H), 1.23-1.27 (m, 3H).

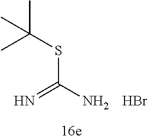

16e: white solid (51% yield). ¹HNMR (400 MHz, DMSO-d₆) δ=9.19 (s, 4H), 1.50 (s, 9H).

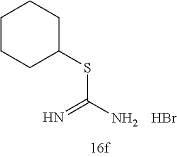

16f: off-white solid (66.7% yield). ¹HNMR (400 MHz, DMSO-d₆) δ=8.98-9.14 (m, 4H), 1.92-1.98 (m, 2H), 1.64-1.72 (m, 2H), 1.53-1.59 (m, 1H), 1.34-1.45 (m, 4H), 1.20-1.28 (m, 1H).

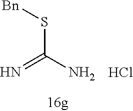 [SMILES: N=C(N)SCc1ccccc1 ; Cl]

16g: white solid (94% yield). ¹HNMR (400 MHz, DMSO-d₆) δ=9.88 (s, 4H), 7.44-7.46 (m, 2H), 7.30-7.43 (m, 3H), 4.56 (s, 2H).

### 4.2 Screening of Pre-Alkylated Isothiourea:

### 4.3 Reaction Procedure:

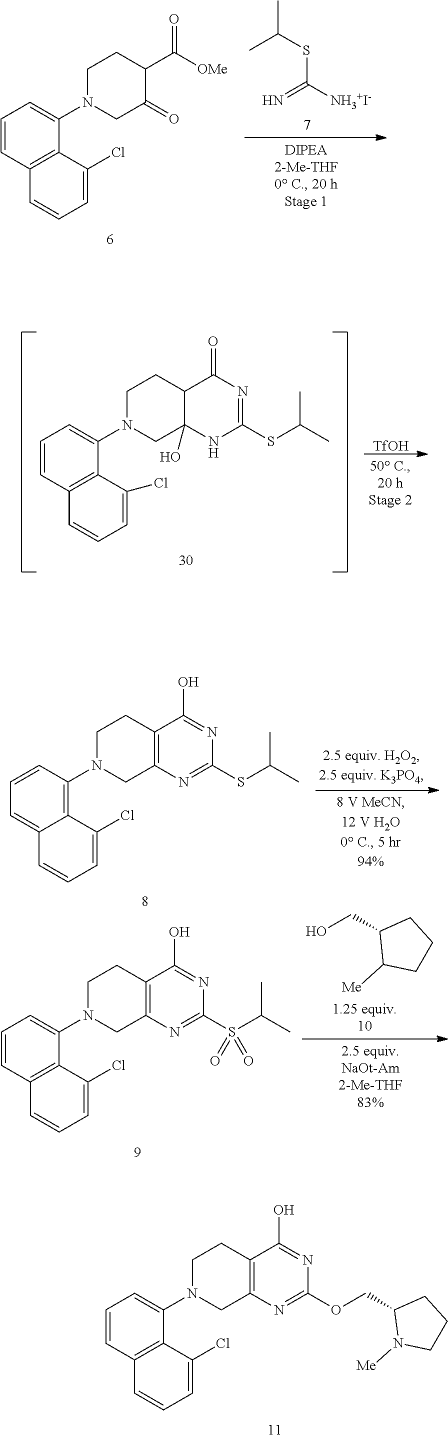

### Synthesis of Compound 8 (Final Compound of Step (e))

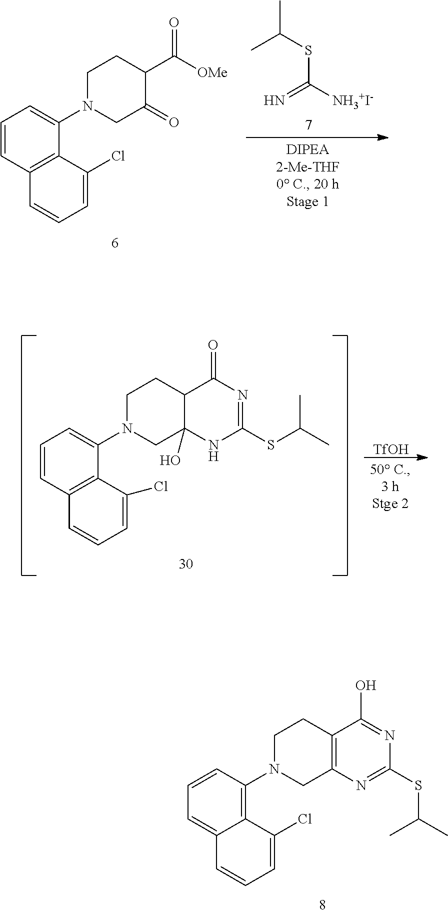

Stage 1: To a solution of ketoester 6 (200 g, 613 mmol) and isopropylisothiouronium iodide 7 (1.1 equiv., 168 g) in 2-MeTHF (2 L) at 0° C. in a 5-L reactor was added DIPEA (1.1 equiv., 117 mL) dropwise over 30 min. The reaction was stirred at 0° C. for 18 h to reach completion and give a white slurry.

Stage 2: TfOH (1.0 equiv., 54 mL) was added dropwise to the slurry at 0° C. in 10 min. The resulting mixture was then heated to 50° C. in 0.5 h and then stirred for 5 hours. Upon the consumption of intermediate, 1.2 L Na₂SO₃(aq., 7 wt %) was added and the reaction mixture stirred at 50° C. for 10 min. The layers were separated and the organic layer was concentrated at 35° C. to give a 1000 mL slurry. The slurry was added with 1500 mL acetonitrile in 2 hours and then was cooled to 0° C. in 2 hours. The resulting slurry was filtered and the remanent was washed with mixed solvent ACN/2-MeTHF (v/v=3/1, 400 mL twice). The solid was dried under vacuum at 35° C. for 18 h to afford the product 8 as a light-yellow solid (223 g, 98.6 wt %, 94% yield, 99.9% purity). ¹H NMR (400 MHz, DMSO-d₆) δ 12.59 (s, 1H), 7.88 (dd, J=8.2, 1.3 Hz, 1H), 7.70 (dd, J=8.2, 1.1 Hz, 1H), 7.54 (dd, J=7.4, 1.3 Hz, 1H), 7.49 (t, J=7.8 Hz, 1H), 7.40 (t, J=7.8 Hz, 1H), 7.31 (dd, J=7.6, 1.2 Hz, 1H), 3.94 (d, J=17.1 Hz, 1H), 3.83 (p, J=6.8 Hz, 1H), 3.69 (dt, J=17.2, 2.2 Hz, 1H), 3.46-3.38 (m, 1H), 3.02 (ddd, J=11.9, 10.0, 4.1 Hz, 1H), 2.78-2.65 (m, 1H), 2.47-2.41 (m, 1H), 1.30 (dd, J=6.9, 5.2 Hz, 6H). ¹³C NMR (101 MHz, DMSO-d₆) δ 162.0, 157.8, 148.1, 137.0, 129.5, 128.9, 128.5, 126.8, 125.8, 124.9, 124.6, 118.7, 114.5, 56.9, 49.6, 35.6, 22.5 (d, J=1.6 Hz), 21.8. HRMS (ESI) calculated for C₂₀H₂₁ClN₃OS: 386.1089[M+H]⁺, Found: 386.1081.

Weak acids like acetic acid could not drive the reaction to completion. Strong acids, such as HCl or MsOH, substantially improved reaction conversion.

### Synthesis of Compound 9 (Final Compound of Step (f))

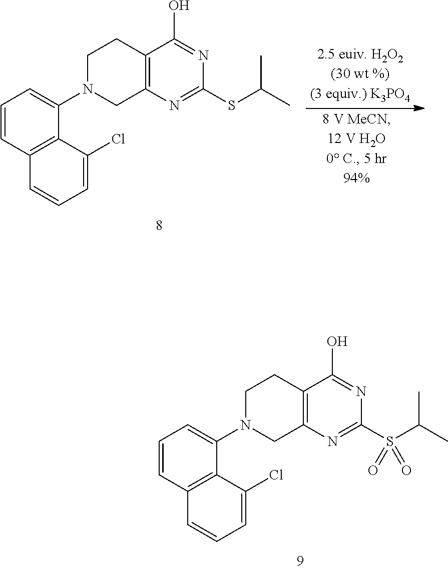 [SMILES: CC(C)Sc1nc(O)c2c(n1)CN(c1cccc3cccc(Cl)c13)CC2 ; CC(C)S(=O)(=O)c1nc(O)c2c(n1)CN(c1cccc3cccc(Cl)c13)CC2]

To a solution of K₃PO₄ (3.0 equiv., 358 g) in water (2.64 L) in a 5-L reactor was added acetonitrile (1.76 L) and sulfide 8 (220 g, 562 mmol). The resulting mixture was then heated to 55° C. in 0.5 h and then stirred for 1 hour. The mixture was then cooled to 20° C. to afford two layers. After discarding the bottom layer, the retained upper layer was cooled to 0° C. and was added with 30% H₂O₂ (2.5 equiv., 143 mL) in 60 min. Upon the consumption of sulfide starting material in 4 hours, 176 mL Na₂SO₃(aq., 21 wt %) was added in 10 min. The resulting mixture was filtered and the filtrate was cooled to 10° C. aq. HCl (320 mL, 1M) was added over 20 min to adjust the mixture pH~5. Seeding with 2 g sulfone and stirring for 2 h afforded a slurry. Aqueous HCl (270 mL, 1 M) was then added dropwise over 20 min to adjust the reaction mixture pH~3. 1.6 L water was then added over 1 hour to afford a yellow slurry, which was filtered and cake was washed with 2×440 mL mixed ACN/H2O solvent (v/v=3/7). The product was dried under vacuum at 30° C. for 24 h to give sulfone 9 as a light-yellow solid (231 g, 95.2 wt %, 94% yield, 99.9% purity). ¹H NMR (400 MHz, DMSO-d₆) δ 13.65 (s, 1H), 7.87 (dd, J=8.3, 1.3 Hz, 1H), 7.71 (dd, J=8.3, 1.1 Hz, 1H), 7.53 (dd, J=7.5, 1.3 Hz, 1H), 7.49 (t, J=7.8 Hz, 1H), 7.40 (t, J=7.8 Hz, 1H), 7.33 (dd, J=7.6, 1.2 Hz, 1H), 4.18 (d, J=17.2 Hz, 1H), 3.95 (dt, J=17.2, 1.8 Hz, 1H), 3.81 (hept, J=6.9 Hz, 1H), 3.55-3.45 (m, 1H), 3.13 (ddd, J=11.9, 10.0, 4.1 Hz, 1H), 2.96 (ddd, J=16.6, 10.1, 6.4 Hz, 1H), 2.72-2.62 (m, 1H), 1.25 (d, J=6.9 Hz, 6H). ¹³C NMR (101 MHz, DMSO-d₆) δ 168.0, 163.5, 159.9, 147.7, 137.0, 129.6, 128.8, 128.6, 126.8, 125.9, 125.0, 124.9, 119.0, 117.3, 57.1, 50.7, 49.2, 22.3, 14.6 (d, J=8.4 Hz). HRMS (ESI) calculated for C₂₀H₂₁ClN₃O₃S: 418.0992[M+H]⁺, Found: 418.0987.

### Synthesis of Compound 11 (Final Compound of Step (g))

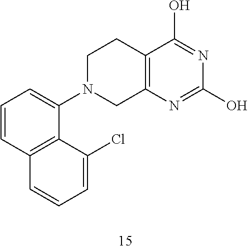 [SMILES: Oc1nc(O)c2c(n1)CN(c1cccc3cccc(Cl)c13)CC2]

To a cooled solution of sulfone 9 (100 g, 95.2% assay, 0.228 mol) and prolinol 10 (32.8 g, 0.284 mol) in 2-MeTHF (800 mL) at 0° C. in a 2-L jacketed reactor was added NaOtAm solution (30 wt %, 0.23 L, 0.57 mol) in 2-MeTHF slowly in 1 h while maintaining reaction temperature NMT 5° C. The reaction mixture was warmed to 20° C. over 10 h and stirred at this temperature for additional 6 h. Upon the consumption of 9, the reaction mixture was then cooled to 0° C. and followed by sequential addition of water (0.5 L) and aq. HCl (2 M, 0.5 L) slowly in 1 h while maintaining reaction temperature NMT 5° C. The mixture was warmed to 20° C. and stirred for 30 minutes. The layers were separated, aq. layer was washed with 2-MeTHF (0.4 L). To the aq. layer was sequentially added 2-MeTHF (1.0 L) and 10 M NaOH (120 mL) to adjust pH to 9. The mixture was heated NMT 45° C. for 1 h. The layers were separated, and aq. layer was discarded. The organic layer was washed with brine solution (0.5 L) and polish filtered while maintaining reaction temperature NLT 40° C. The acetonitrile (1.0 L) and 1 wt % seeds were charged. After 3 hours agitation at 45° C., the mixture was concentrated to 1.0 L. Additional acetonitrile (1.0 L) was charged and concentrated back to 1.0 L (twice) to give a thick slurry. The slurry was allowed to cool to 20° C., filtered and washed with acetonitrile (2×0.20 L). The product was dried under vacuum NMT 40° C. for 16 h to give 11 as an off-solid in 83% yield (80.2 g, 98.3 wt %, 99.47% purity). ¹H NMR (400 MHz, CDCl₃) δ 7.72 (dd, J=8.2, 1.3 Hz, 1H), 7.58 (dd, J=8.2, 1.2 Hz, 1H), 7.51 (dd, J=7.4, 1.3 Hz, 1H), 7.42 (t, J=7.8 Hz, 1H), 7.31 (t, J=7.8 Hz, 1H), 7.22 (dt, J=7.6, 1.2 Hz, 1H), 4.34 (t, J=4.7 Hz, 2H), 4.11 (d, J=17.5 Hz, 1H), 3.71 (dq, J=17.5, 2.0 Hz, 1H), 3.59-3.49 (m, 1H), 3.11 (tt, J=11.2, 4.0 Hz, 2H), 2.98-2.85 (m, 1H), 2.70-2.57 (m, 2H), 2.46 (d, J=1.7 Hz, 3H), 2.34-2.23 (m, 1H), 2.05-1.91 (m, 1H), 1.89-1.78 (m, 1H), 1.78-1.64 (m, 2H) ppm. ¹³C NMR (101 MHz, CDCl₃) δ 165.0 (d, J=1.9 Hz), 159.9, 155.2, 148.8, 137.5, 130.4, 129.7, 128.3, 126.5, 126.2, 125.6, 124.9, 118.6, 113.0, 77.4, 69.9 (d, J=18.2 Hz), 63.9 (d, J=3.9 Hz), 57.6 (d, J=1.8 Hz), 50.3 (d, J=2.7 Hz), 41.7 (d, J=8.9 Hz), 28.3 (d, J=2.3 Hz), 23.1 (d, J=4.0 Hz), 21.9 ppm.

### Compound 15:

¹H NMR (400 MHz, DMSO-d₆) δ 10.98 (s, 2H), 7.90 (d, J=1.3 Hz, 1H), 7.74 (d, J=8.1 Hz, 1H), 7.59 (dd, J=7.5, 1.2 Hz, 1H), 7.52 (t, J=7.8 Hz, 1H), 7.44 (t, J=7.8 Hz, 1H), 7.31 (dd, J=7.6, 1.2 Hz, 1H), 3.86 (d, J=17.0 Hz, 1H), 3.52 (d, J=17.0 Hz, 1H), 3.39 (ddd, J=12.3, 5.7, 2.6 Hz, 1H), 3.08 (ddd, J=11.8, 9.9, 4.3 Hz, 1H), 2.51 (qt, J=7.0, 3.7 Hz, 2H), 2.32 (d, J=16.1 Hz, 1H) ppm. ¹³C NMR (101 MHz, DMSO-d₆) δ 164.0, 151.0, 147.7, 147.2, 137.0, 129.7, 128.8, 128.6, 126.8, 126.0, 125.0, 125.0, 119.6, 103.8, 52.3, 49.3, 20.9 ppm.

### Decomposition of Compound 11 During Crystallization

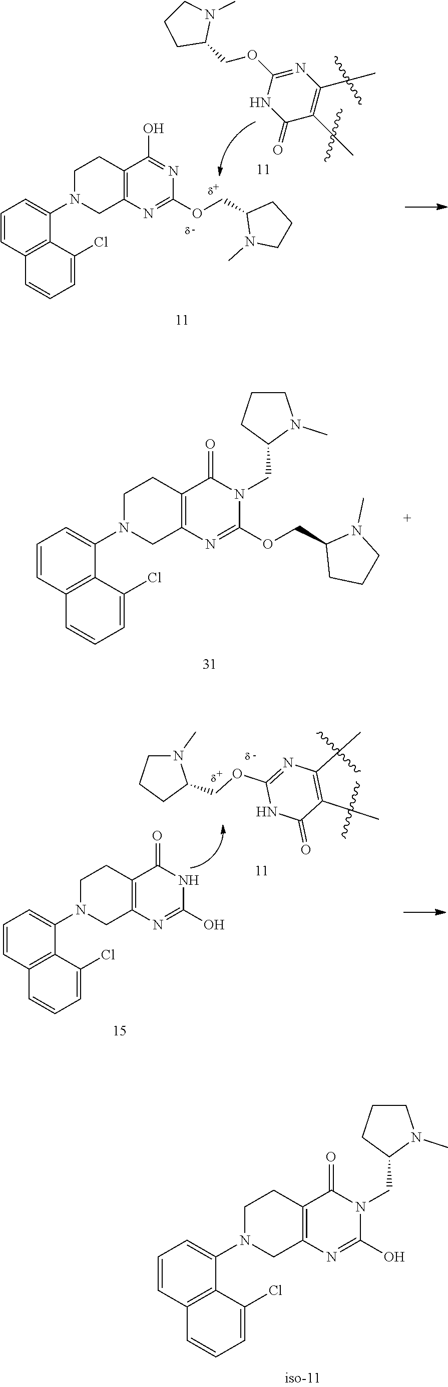

A suspension of isolated intermediate 11 (1.0 g) in 20 mL of mixture of 2-MeTHF/ACN/water (1:1:0.1) heated at 55° C. for 1 week. The degradation of 120 was monitored with HPLC. The data is presented below.

LCAP

| Time | 11 | 31 | 15 | Iso-11 |
|---|---|---|---|---|
| 22 h | 71.1 | 13.5 | 13.2 | 2.2 |
| 66 h | 34.9 | 23.4 | 27.5 | 9.4 |
| 168 h | 22.3 | 38.1 | 3.4 | 24.6 |

### Compound 31:

¹H NMR (400 MHz, CDCl₃) δ 7.74 (d, J=7.88 Hz, 1H), 7.59 (d, J=8.00 Hz, 1H), 7.52 (d, J=6.88 Hz, 1H), 7.44 (t, J=7.82 Hz, 1H), 7.33 (t, J=7.75 Hz, 1H), 7.25 (d, J=7.50 Hz, 1H), 4.44 (td, J=10.54, 4.82 Hz, 1H), 4.23 (ddd, J=13.29, 10.91, 6.32 Hz, 1H), 4.12 (br d, J=4.13 Hz, 2H), 3.65-3.75 (m, 1H), 4.06 (br s, 1H), 3.52-3.61 (m, 1H), 3.13-3.24 (m, 1H), 3.09-3.13 (m, 1H), 3.08 (br d, J=7.75 Hz, 1H), 2.90-2.99 (m, 1H), 2.73 (br d, J=4.00 Hz, 1H), 2.63 (br s, 1H), 2.60 (br s, 1H), 2.53 (d, J=5.88 Hz, 3H), 2.45 (d, J=1.63 Hz, 3H), 2.30-2.35 (m, 1H), 2.28 (br s, 1H), 2.05 (br d, J=8.38 Hz, 1H), 1.87 (br d, J=2.75 Hz, 2H), 1.79-1.86 (m, 2H), 1.73-1.79 (m, 2H), 1.72 (br s, 1H) ppm. ¹³C NMR (101 MHz, CDCl₃) δ 163.0, 156.7, 154.3, 148.7, 137.4, 130.2, 129.5, 128.2, 126.2, 126.3, 126.0, 125.5, 124.7, 118.4, 112.4, 70.7, 63.8, 63.7, 57.5, 67.2, 57.1, 50.3, 45.0, 41.5, 41.2, 29.5, 29.0, 22.8, 22.7, 22.4 ppm.

Compound iso-11:

¹H NMR (400 MHz, CDCl₃) δ 7.74 (dd, J=8.13, 1.25 Hz, 1H), 7.62 (d, J=8.13 Hz, 1H), 7.48-7.55 (m, 1H), 7.43 (t, J=7.82 Hz, 1H), 7.29-7.34 (m, 1H), 7.22 (d, J=7.50 Hz, 1H), 3.96-4.10 (m, 1H), 3.80-3.96 (m, 2H), 3.63 (br dd, J=16.76, 1.50 Hz, 1H), 3.50 (br dd, J=11.76, 3.88 Hz, 1H), 3.05-3.13 (m, 1H), 2.87-3.02 (m, 1H), 2.76-2.87 (m, 1H), 2.50-2.57 (m, 1H), 2.47 (br dd, J=6.19, 3.31 Hz, 1H), 2.23 (d, J=6.25 Hz, 3H), 1.99-2.13 (m, 1H), 1.67-1.80 (m, 2H), 1.58-1.66 (m, 1H), 1.39-1.58 (m, 1H) ppm. ¹³C NMR (101 MHz, CDCl₃) δ 163.4, 153.3, 147.8, 145.0, 137.3, 129.8, 128.7, 128.3, 126.3, 125.9, 125.7, 125.5, 118.9, 106.0, 64.0, 56.9, 52.2, 50.1, 43.9, 40.7, 29.7, 22.4, 21.6 ppm.

### An Alternative General Reaction Procedure:

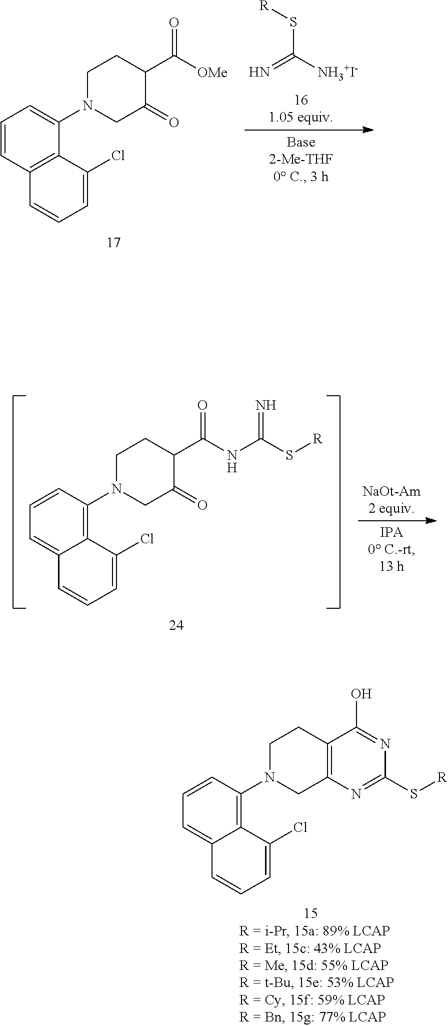

Stage 1: Under nitrogen, to isothiourea (1.05 equiv., 5 g) in 2-Me-THF(75 mL) at 0-5° C. base(1.5 equiv.) was added dropwise, keeping the reaction temperature at 0-5° C. The mixture was stirred at 0° C. for 10 min followed by addition of ketoester 17 (1.0 equiv.). The resulting slurry was stirred at 0° C. for 3 h or until the completion of the reaction.

Stage 2: The above crude mixture was dissolved in anhydrous IPA (50 mL) to give a slurry and cooled to 0° C. Sodium tert-pentoxide (2 equiv.) was added slowly, keeping the reaction temperature<5 CC. The reaction was warmed to room temperature and stirred at this temperature for 13 h or until the completion of the reaction. H₂O (20 mL) was added to give a hazy solution which was polish filtered through celite to give a clear solution. HOAc/IPA (1/1) was added dropwise until the reaction solution became hazy. A fine slurry was obtained upon seeding and continuous stirring. Filtration of the slurry and cake wash with IPA/H₂O=3/7 (10 mL) afforded the desired sulfide products. 15c, 15d, 15e, 15f, 15g. These products were used directly, except 15g was further purified by chromatography.

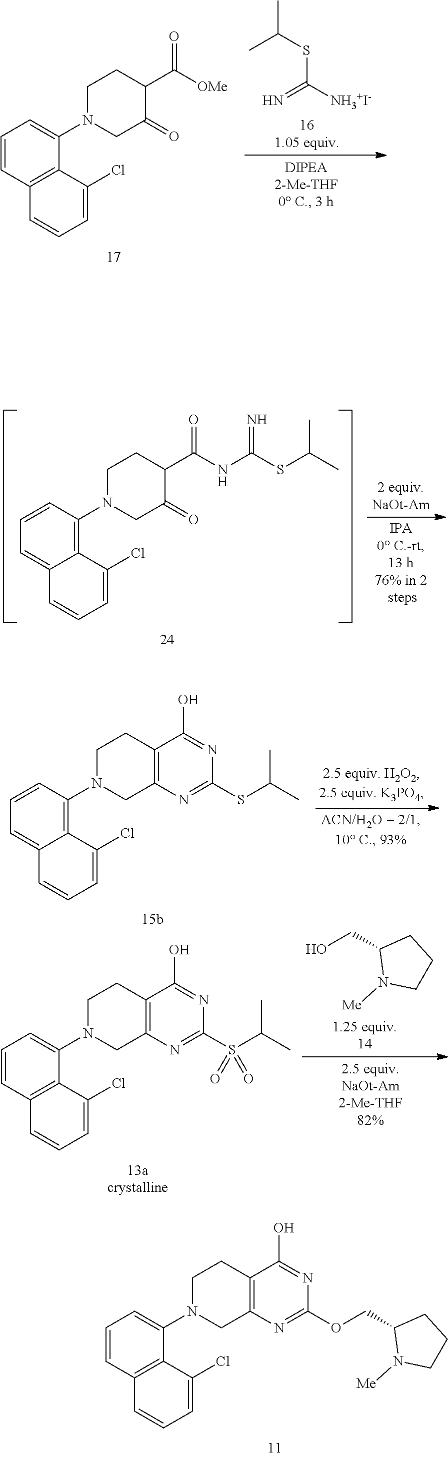

### Synthesis of 15b:

Stage 1: To a solution of 16 (1.05 equiv., 154 g) in 1000 mL 2-Me-THF at 0° C. in 5-L reactor 1.5 equiv. of DIPEA was added dropwise over 1 h to give a white slurry. Ketoester 17 (200 g, 95% purity) was added slowly in 15 min followed by rinse with 600 mL 2Me-THF. The resulting slurry was stirred at 0° C. for 20 h. 400 mL water was added followed by addition of aqueous HCl (1M, 360 mL) dropwise in 20 min to afford a white slurry (pH=3). The slurry was warmed to 20° C. to give a clear biphasic mixture. The aqueous solution (1.2 L) was discarded. The organic layer (1600 mL) was concentrated to 500 mL and azeotropically exchanged with 2×400 mL IPA to give a yellow slurry (~500 mL in volume).

Stage 2: 2 L isopropanol was added to the above slurry and the mixture was cooled to 0° C. 2 eq NaAmylate was added slowly to the mixture and rinsed with 200 mL isopropanol. The reaction mixture was stirred at 0° C. for 0.5 h and then warmed to 25° C. over 0.5 h. The yellow slurry was stirred for 15 h followed by addition of 800 mL H₂O at 20° C. to give a hazy solution. This solution was polish filtered through celite to give a clear solution. The solution was quenched by addition of 150 mL HOAc/isopropanol (1/1) in 20 min. The solution (pH=7.02) turned to hazy and upon seeding (2 g seed) formed a thick slurry after 3 h. The slurry was filtered and the cake was washed with 2×200 mL isopropanol/120 (V/V=1/1), and 200 mL isopropanol sequentially, dried at 45° C. for 15 h to afford sulfide 15b as a free-flow pale yellow solid (182.8 g, 93.7% purity, 76% yield).

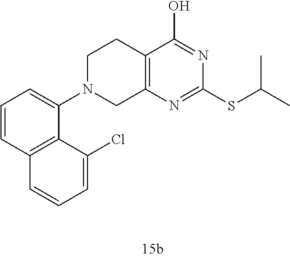 [SMILES: CC(C)Sc1nc(O)c2c(n1)CN(c1cccc3cccc(Cl)c13)CC2]

15b: light yellow solid (182.8 g, 93.7% purity, 76% yield). R_{f}=0.69 (dichloromethane/methanol=10/1). ¹H NMR (400 MHz, DMSO-d₆) δ 12.60 (s, 1H), 7.88 (dd, J=8.2, 1.3 Hz, 1H), 7.70 (dd, J=8.3, 1.1 Hz, 1H), 7.54 (dd, J=7.5, 1.3 Hz, 1H), 7.49 (t, J=7.8 Hz, 1H), 7.40 (t, J=7.8 Hz, 1H), 7.31 (dd, J=7.6, 1.2 Hz, 1H), 3.95 (d, J=17.1 Hz, 1H), 3.84 (p, J=6.9 Hz, 1H), 3.69 (dt, J=17.2, 2.2 Hz, 1H), 3.46-3.38 (m, 1H), 3.02 (ddd, J=11.8, 10.0, 4.1 Hz, 1H), 2.78-2.65 (m, 1H), 2.49-2.42 (m, 1H), 1.31 (dd, J=6.9, 5.1 Hz, 6H). ¹³C NMR (101 MHz, DMSO-d₆) δ 162.0, 157.8, 157.8, 148.1, 137.0, 129.5, 128.9, 128.5, 126.7, 125.8, 124.9, 124.6, 118.7, 114.5, 56.9, 49.6, 35.5, 22.5 (d, J=1.6 Hz), 21.8. HRMS (ESI) calculated for C₂₀H₂₁ClN₃OS: 386.1089[M+H]⁺, Found: 386.1081.

### Synthesis of 13a:

To a 5-L reactor the following were added sequentially: H₂O (560 mL), K₃PO₄ (2.5 equiv., 191 g), sulfide 15 (140 g, 97% purity), and ACN (1.12 L). The mixture was heated to 35° C. for 15 min to dissolve sulfide and resulted in a biphasic mixture. Phase cut to discard the bottom layer (370 mL) and the result organic layer was polish filtered. To the filtrate (pH=12.2) cooled at 10° C. was added 30 wt % H₂O₂ (2.5 eq, 90.2 mL) in 1 h, keeping the reaction temperature between 8-11° C. The mixture was stirred at 10° C. for 25 h followed by addition of aq. HCl (220 mL, 1M) dropwise over 15 min. The reaction temperature was raised to 20° C. to give a hazy solution (pH=5.3). Seeding with 1 g sulfone and stirring for 2 h afforded a slurry. To the slurry was sequentially added 150 mL aqueous HCl (1 M) dropwise over 10 min (reaction mixture at pH=1.7) and 720 mL water over 20 min. The slurry was filtered and cake was washed with 2×140 mL ACN/H₂O=3/7 mixture. The product was dried under vacuum at ambient temperature for 48 h to give sulfone 13a as a light-yellow solid (142 g, 98.6% purity, 94% yield).

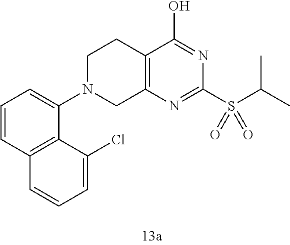 [SMILES: CC(C)S(=O)(=O)c1nc(O)c2c(n1)CN(c1cccc3cccc(Cl)c13)CC2]

13a: light yellow solid (142 g, 98.6% purity, 94% yield). R_{f}=0.47 (dichloromethane/methanol=10/1). ¹H NMR (400 MHz, DMSO-d₆) δ 13.65 (s, 1H), 7.92 (dd, J=8.1, 1.3 Hz, 1H), 7.75 (dd, J=8.3, 1.1 Hz, 1H), 7.57 (d, J=6.1 Hz, 1H), 7.54 (t, J=7.8 Hz, 1H), 7.44 (t, J=7.8 Hz, 1H), 7.38 (dd, J=7.6, 1.2 Hz, 1H), 4.20 (d, J=17.2 Hz, 1H), 4.00 (dt, J=17.4, 1.8 Hz, 1H), 3.82 (hept, J=6.8 Hz, 1H), 3.60-3.50 (m, 1H), 3.20 (ddd, J=12.0, 10.1, 4.1 Hz, 1H), 3.05-2.92 (m, 1H), 2.72-2.65 (m, 1H), 1.26 (d, J=6.9 Hz, 6H). ¹³C NMR (101 MHz, DMSO-d₆) δ 168.0, 163.5, 160.0, 147.7, 137.0, 129.6, 128.8, 128.6, 126.8, 125.9, 125.0, 124.9, 119.0, 117.3, 57.1, 50.6, 49.2, 22.3, 14.6 (d, J=8.4 Hz). HRMS (ESI) calculated for C₂₀H₂₁ClN₃O₃S: 418.0992[M+H]⁺, Found: 418.0987.

### Synthesis of 11:

To a cooled solution of sulfone 13a (140 g, 98.6% purity) and prolinol 14 (1.25 equiv., 46.6 g) in 2-Me-THF (1.4 L) at 0° C. in a 5-L reactor was added Nat-Amylate solution (30 wt %, 0.33 L) in 2-MeTHF slowly in 1 h. The reaction mixture was warmed to room temperature and stirred at this temperature for 15 h. The reaction mixture was then cooled to 0° C. and followed by sequential addition of H₂O (1.4 L) and aq. HCl (2 M, 0.64 L) slowly in 15 min. The mixture was warmed to room temperature and layers were separated. The aq layer was extracted with 2-Me-THF (1.2 L). To the combined organic layers was added 10 M NaOH (140 mL) to adjust pH to 8 during which period a precipitation initiated. H₂O (0.42 L) was added to afford a thick slurry. 2-MeTHF (2.1 L) was added to the slurry and the mixture was heated to 60° C. and stirred at this temperature for 1 h. The aqueous layer was discarded and the organic layer was polish filtered. The filtrate was concentrated to 1.2 L and then diluted with 1.2 L acetonitrile. The mixture was charged with 3 wt % seeds and heated to 40° C. After 3 hours stirring, the mixture was concentrated to 1.2 L. Another 1.2 L acetonitrile was added concentration again to 1.2 L to give a thick slurry. The slurry was filtered and washed with 2×0.28 L ACN. The product was dried under vacuum at room temperature for 18 h to give 11 as a light-yellow solid (113 g, 97.1% purity, 82% yield).

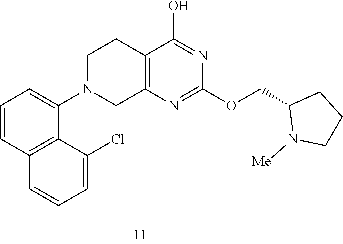 [SMILES: CN1CCC[C@H]1COc1nc(O)c2c(n1)CN(c1cccc3cccc(Cl)c13)CC2]

11: gray solid (113 g, 97.1% purity, 82% yield). R_{f}=0.13 (dichloromethane/methanol=10/1). ¹H NMR (400 MHz, CDCl₃) δ 7.72 (dd, J=8.2, 1.3 Hz, 1H), 7.58 (dd, J=8.2, 1.2 Hz, 1H), 7.51 (dd, J=7.4, 1.3 Hz, 1H), 7.42 (t, J=7.8 Hz, 1H), 7.31 (t, J=7.8 Hz, 1H), 7.22 (dt, J=7.6, 1.2 Hz, 1H), 4.34 (t, J=4.7 Hz, 2H), 4.11 (d, J=17.5 Hz, 1H), 3.71 (dq, J=17.5, 2.0 Hz, 1H), 3.59-3.49 (m, 1H), 3.11 (tt, J=11.2, 4.0 Hz, 2H), 2.98-2.85 (m, 1H), 2.70-2.57 (m, 2H), 2.46 (d, J=1.7 Hz, 3H), 2.34-2.23 (m, 1H), 2.05-1.91 (m, 1H), 1.89-1.78 (m, 1H), 1.78-1.64 (m, 2H). ¹³C NMR (101 MHz, CDCl₃) δ 165.0 (d, J=1.9 Hz), 159.9, 155.2, 148.8, 137.5, 130.4, 129.7, 128.3, 126.5, 126.2, 125.6, 124.9, 118.6, 113.0, 77.4, 69.9 (d, J=18.2 Hz), 63.9 (d, J=3.9 Hz), 57.6 (d, J=1.8 Hz), 50.3 (d, J=2.7 Hz), 41.7 (d, J=8.9 Hz), 28.3 (d, J=2.3 Hz), 23.1 (d, J=4.0 Hz), 21.9. HRMS (ESI) calculated for C₂₃H₂₆ClN₄O₂: 425.1739 [M+H]⁺, Found: 425.1740.

HRMS (ESI) calculated for C₂₃H₂₆ClN₄O₂: 425.1739 [M+H]⁺, Found: 425.1740.

### Example 3

### 3.1 Reaction Procedure

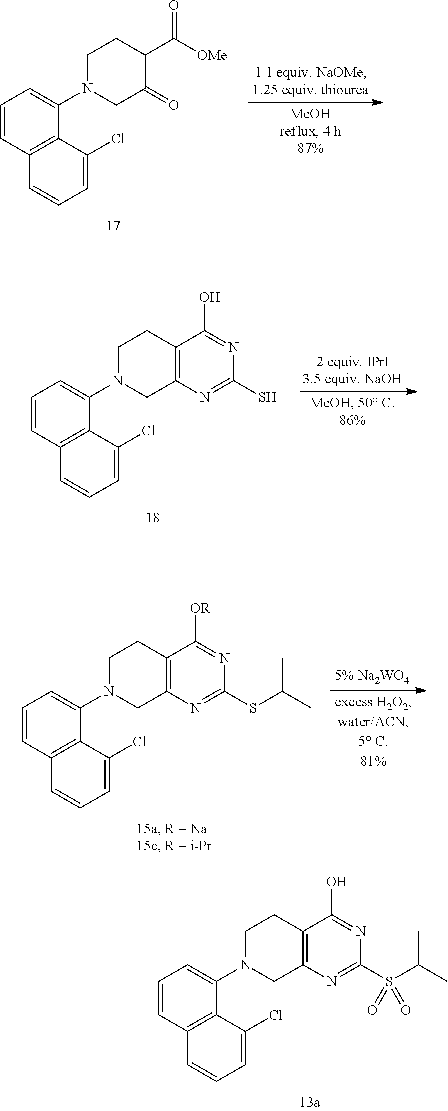

### Synthesis of 18:

To a mixture of ketoester 17 (100 g, 95% purity) and thiourea (1.25 equiv., 64 g) in MeOH (1 L) in a 2-L reactor was added sodium methoxide solution (25 wt %, 192 mL) slowly. The reaction was heated under reflux for 4 h and cooled to 0-5° C. Conc. HCl was charged slowly to the reaction mixture until pH to 3-4, during which the product precipitated out to give a slurry. The slurry was filtered, the cake was washed with MeOH (0.5 L) and triturated with water (1 L) at 60° C. for 1 hr. The slurry was filtered and the cake was washed with water (0.5 L). The product was dried under vacuum at 60° C. for 18 h until thermogravimetric analysis (TGA) showed <0.2% weight loss to afford thiol 18 as a pale-yellow solid (95 g, 94% purity, 87% yield).

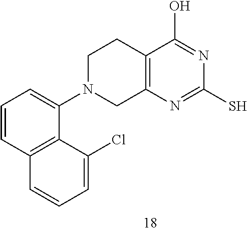 [SMILES: Oc1nc(S)nc2c1CCN(c1cccc3cccc(Cl)c13)C2]

18: Pale yellow solid (95 g, 94% purity, 87% yield). R_{f}=0.67 (dichloromethane/methanol=10/1).

¹H NMR (400 MHz, DMSO-d₆) δ 12.46 (s, 1H), 12.32 (s, 1H), 7.90 (d, J=8.1 Hz, 1H), 7.74 (d, J=8.1 Hz, 1H), 7.58 (d, J=7.4 Hz, 1H), 7.52 (t, J=7.8 Hz, 1H), 7.43 (t, J=7.8 Hz, 1H), 7.30 (d, J=7.5 Hz, 1H), 3.96 (d, J=17.4 Hz, 1H), 3.57 (d, J=17.4 Hz, 1H), 3.38 (m, 1H), 3.09 (td, J=11.0, 4.2 Hz, 1H), 2.61-2.50 (m, 2H), 2.35 (d, J=16.6 Hz, 1H).

¹³C NMR (101 MHz, DMSO-d₆) δ 174.2, 160.9, 147.5, 147.5, 137.0, 129.7, 128.7, 128.7, 126.8, 126.0, 125.2, 125.0, 119.4, 109.8, 52.0, 48.9, 21.0.

HRMS (ESI) calculated for C₁₇H₁₅ClN₃OS: 344.0624 [M+H]⁺, Found: 344.0620.

### Synthesis of 15a:

To a 2-L reactor was charged with sulfide 18 (65 g, 94% purity), MeOH (195 mL), and 3M NaOH (3.5 equiv.) and the mixture was stirred to a solution. Isopropyl iodide (2 equiv.) was added to the solution in one portion. The resulting mixture was heated to 50° C. and stirred at this temperature for 2 h. 2-MeTHF (650 mL) and water (260 mL) were added to the reaction. The mixture was cooled to 17-20° C. and layers were separated. The bottom aqueous layer was discarded and the top organic layer was washed with 325 mL NaOH (0.5M) three times. The resulting organic layer was concentrate to ~100 mL and added slowly to a mixture of 650 mL DCM and 325 mL aqueous NaOH (0.5 M). After stirring for 17 h, the slurry was filtered and washed with 325 mL aq. Na₂CO₃ (0.15 M). The wet cake was dried under vacuum at 40° C. for 22 h to afford sulfide 15a as a pale-yellow solid (66 g, 94% purity, 86% yield).

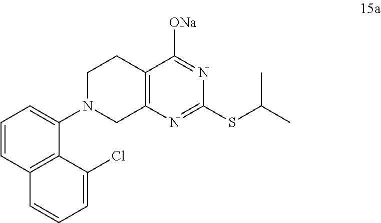

15a: light yellow solid (66 g, 94% purity, 86% yield). R_{f}=0.69 (dichloromethane/methanol=10/1).

¹H NMR (400 MHz, DMSO-d₆) δ δ 7.87 (dd, J=8.2, 1.1 Hz, 1H), 7.66 (d, J=7.6 Hz, 1H), 7.54 (dd, J=7.4, 1.2 Hz, 1H), 7.49 (t, J=7.8 Hz, 1H), 7.45-7.37 (m, 1H), 7.28 (dd, J=7.6, 0.8 Hz, 1H), 3.84 (d, J=15.9 Hz, 1H), 3.74 (p, J=6.8 Hz, 1H), 3.56 (d, J=15.9 Hz, 1H), 3.46-3.38 (m, 1H), 3.00 (ddd, J=11.7, 9.9, 4.3 Hz, 1H), 2.63 (ddd, J=15.9, 9.9, 5.8 Hz, 1H), 2.37 (d, J=16.4 Hz, 1H), 1.27 (dd, J=6.8, 1.7 Hz, 1H).

¹³C NMR (101 MHz, DMSO-d6) δ 172.3, 165.5, 156.4, 149.1, 137.1, 129.2, 129.1, 128.4, 126.8, 125.7, 124.9, 123.8, 118.0, 110.3, 57.2, 51.3, 33.5, 23.5, 22.8.

HRMS (ESI) calculated for C₂₀H₂₁ClN₃OS: 386.1089[M-Na+2H]+, Found: 386.1081.

### Synthesis of 13a:

To a 100-mL reactor was charged with sulfide 15a (5 g, 94% purity), Na₂WO₄·2H₂O (0.05 equiv., 0.19 g), H₂O (25 mL), and CH₃CN (50 mL). The mixture was stirred to give a solution and cooled to 5° C. H₂O₂ (30 wt %, 3 equiv., 3.52 mL) was added over 5 h and the mixture was stirred at 5° C. for 12 h. The reaction was quenched with aq. NaHSO₃ (1 equiv., 1.2 g NaHSO₃ in 20 mL Water) to pH ~7. CH₃CN was removed via distillation at approx. 40° C. followed by slow addition of AcOH (1.5 equiv., 0.98 mL) at room temperature during which a precipitate forms. The slurry was filtered and washed with 2×25 mL water. The cake was dried at 40° C. overnight to give yellow solid (3.89 g, 97% purity, 81% yield).

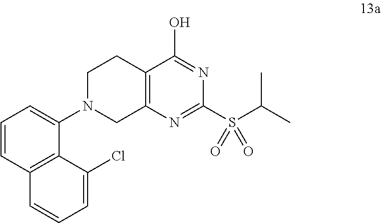 [SMILES: CC(C)S(=O)(=O)c1nc(O)c2c(n1)CN(c1cccc3cccc(Cl)c13)CC2]

13a: light yellow solid (3.89 g, 97% purity, 81% yield). R_{f}=0.47 (dichloromethane/methanol=10/1).

¹H NMR (400 MHz, DMSO-d₆) δ 13.65 (s, 1H), 7.92 (dd, J=8.1, 1.3 Hz, 1H), 7.75 (dd, J=8.3, 1.1 Hz, 1H), 7.57 (d, J=6.1 Hz, 1H), 7.54 (t, J=7.8 Hz, 1H), 7.44 (t, J=7.8 Hz, 1H), 7.38 (dd, J=7.6, 1.2 Hz, 1H), 4.20 (d, J=17.2 Hz, 1H), 4.00 (dt, J=17.4, 1.8 Hz, 1H), 3.82 (hept, J=6.8 Hz, 1H), 3.60-3.50 (m, 1H), 3.20 (ddd, J=12.0, 10.1, 4.1 Hz, 1H), 3.05-2.92 (m, 1H), 2.72-2.65 (m, 1H), 1.26 (d, J=6.9 Hz, 6H).

¹³C NMR (101 MHz, DMSO-d₆) δ 168.0, 163.5, 160.0, 147.7, 137.0, 129.6, 128.8, 128.6, 126.8, 125.9, 125.0, 124.9, 119.0, 117.3, 57.1, 50.6, 49.2, 22.3, 14.6 (d, J=8.4 Hz).

HRMS (ESI) calculated for C₂₀H₂₁ClN₃O₃S: 418.0992[M+H]⁺, Found: 418.0987.

### Example 4

### Section 5. Synthesis of 11 Through Triphosgene Route

### 5.1 Reaction Procedure:

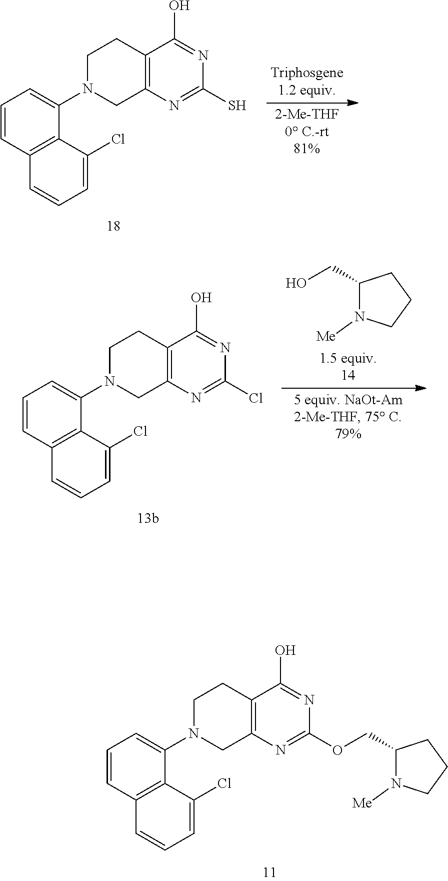 [SMILES: Oc1nc(S)nc2c1CCN(c1cccc3cccc(Cl)c13)C2 ; Oc1nc(Cl)nc2c1CCN(c1cccc3cccc(Cl)c13)C2 ; CN1CCC[C@H]1CO ; CN1CCC[C@H]1COc1nc(O)c2c(n1)CN(c1cccc3cccc(Cl)c13)CC2]

### Synthesis of 13b:

To a solution of thiol 18 (15 g) in 2-Me-THF (300 mL) at 0-5° C. was added 10.1 mL HCl (4 M in dioxane) solution slowly and followed with the addition of triphosgene (1.2 equiv., 14.5 g). The reaction mixture was warmed to 25° C. and stirred at this temperature for 15 h, wh to afford a slurry. The slurry was filtered and the cake was rinsed with 75 mL ACN, followed by 75 mL MTBE. The product was dried under vacuum at room temperature for 18 h to afford chloride 13b as a pale-yellow solid (13.5 g, 93.4% purity, 81% yield).

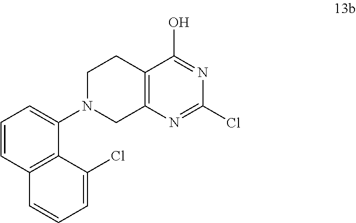 [SMILES: Oc1nc(Cl)nc2c1CCN(c1cccc3cccc(Cl)c13)C2]

13b: white solid (13.5 g, 93.4% purity, 81% yield). R_{f}=0.58 (dichloromethane/methanol=10/1). ¹H NMR (400 MHz, DMSO-d₆) δ 7.89 (dd, J=8.2, 1.3 Hz, 1H), 7.72 (dd, J=8.2, 1.1 Hz, 1H), 7.55 (dd, J=7.4, 1.3 Hz, 1H), 7.51 (t, J=7.8 Hz, 1H), 7.42 (t, J=7.8 Hz, 1H), 7.33 (dd, J=7.6, 1.2 Hz, 1H), 4.00 (d, J=17.4 Hz, 1H), 3.78 (dt, J=17.4, 2.1 Hz, 1H), 3.50-3.40 (m, 1H), 3.14-3.04 (m, 1H), 2.83-2.70 (m, 1H), 2.56-2.46 (m, 1H). 13C NMR (101 MHz, DMSO-d₆) δ 165.0, 160.8, 148.9, 147.9, 137.1, 129.7, 129.0, 128.7, 126.8, 126.0, 125.1, 125.0, 119.1, 116.2, 56.7, 49.4, 21.9. HRMS (ESI) calculated for C₁₇H₁₄C₁₂N₃O: 346.0509 [M+H]⁺, Found: 346.0491.

### Synthesis of 11:

To chloride 13b (41 g, 93% purity) in 2-Me THF (205 mL) at 0° C. was added prolinol 5 (1.5 equiv., 17.8 g) and Nat-Amylate (5 equiv., 56.7 g) sequentially and the reactor was rinsed with 2-Me THE (205 mL). The mixture was heated to 75° C. and stirred at this temperature for 48 h. The reaction mixture was cooled to 0° C. followed by addition of H₂O (0.41 L) and aq. HCl (2M, 0.24 L). The bottom aqueous layer was separated and extracted with 2-Me-THE (0.41 L). To the combined organic layers was added NaOH aqueous solution (10M, 140 mL). Precipitation initiated at pH 8. 2-MeTHF (0.41 L) was added to the mixture which was heated to 55° C. and stirred at this temperature for 1 h. The biphasic mixture was separated and the bottom aqueous layer was extracted by 2-MeTHF (0.41 L). The two organic layers were combined and concentrated to 0.41 L followed by addition of 0.2 L acetonitrile. The mixture was seeded with 4 wt % seeds and stirred at room temperature for 15 h. The resulting slurry was filtered and washed with 2×0.2 L ACN. The product was dried under vacuum at room temperature for 18 h to give 11 as a light-yellow solid (41.9 g, 94.8% purity, 79% yield).

The product NMR data is in accordance with the aforementioned 11 NMR data.

### Example 5

### Synthesis of 13 Through Activation/S_{N}Ar Sequence

### 6.1 General Reaction Procedure:

Stage 1: To a mixture of 11 (1 g, 97.1% purity) and DIPEA (2.5 equiv., 1 mL) in DCM (7 mL) at 0° C. was added 1.1 equiv. activation reagent (ArSO₂Cl/Tf₂O/MsCl) and 0.05 equiv. DMAP sequentially. The reaction mixture was stirred at 0° C. for 15 min and warmed to room temperature. H₂O (3 mL) was added to the reaction mixture and the bottom organic layer was separated. The top organic layer was dried (over MgSO₄) and concentrated to afford crude intermediate 25 which is used directly in the next stage.

Stage 2: A mixture of crude intermediate 25, piperazine (1.5 equiv.), K₃PO₄(3 equiv.) in DMAc (3 mL) was stirred at room temperature for 2 h.

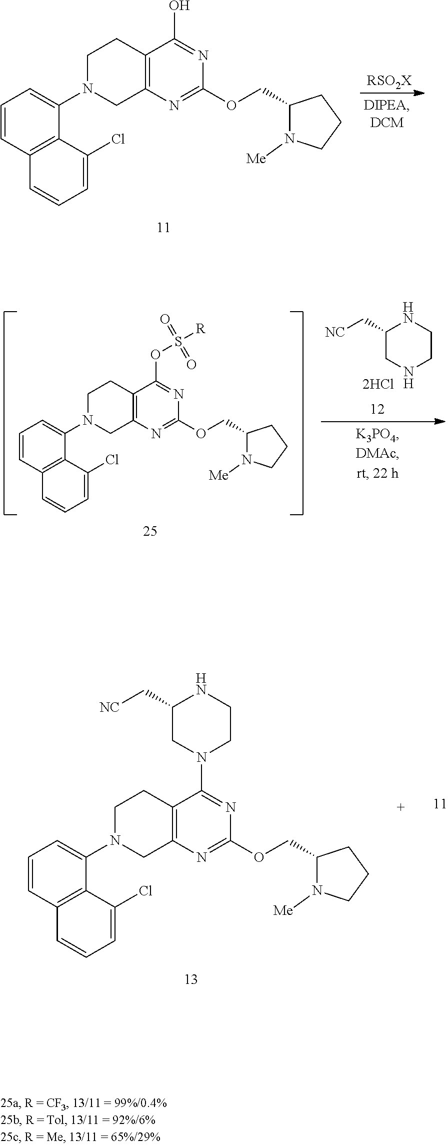

### Example 5A

### 6.2 Synthesis of 13 Through Triflate:

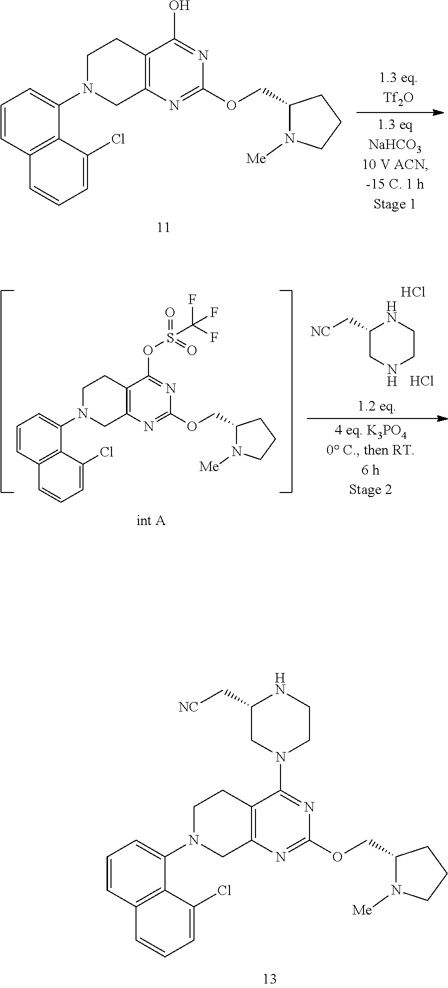

Stage 1: To a mixture of 11 (40 g, 97.2% assay, 91.5 mmol) and NaHCO₃ (10.0 g, 119 mmol) in ACN (400 mL) at −15° C. was added Tf₂O dropwise (27.8 g, 137 mmol). The reaction mixture was stirred at −15° C. for 1 hour.

Stage 2: To the resulting mixture in Stage 1, piperazine side chain (88.9 g, 110 mmol) and K₃PO₄ (77.7 g, 366 mmol) was added sequentially at −15° C. Then, warm the reaction to room temperature for 1 hour. The mixture was stirred at room temperature for 6 hours. To the resultant slurry was added 10% KOH solution (240 mL) and stir for 10 min. The top organic layer was separated followed by addition of H₂O (500 mL) until the mixture turned hazy. 0.3 wt % seeds were added to the mixture. Then, the mixture was stirred at room temperature for 18 hours. H₂O (500 mL) was added slowly over 2 hours to afford a white slurry. The slurry was filtered, and the wet cake was washed with 120 mL ACN/H₂O=1/3 (V/V) mixture and 100 mL H₂O sequentially. The wet cake was dried under vacuum at 40° C. for 18 hours to give product 13 as a white solid (50 g, 89.0 wt %, 91.5% yield, 99.5% purity).

### An Alternative Procedure:

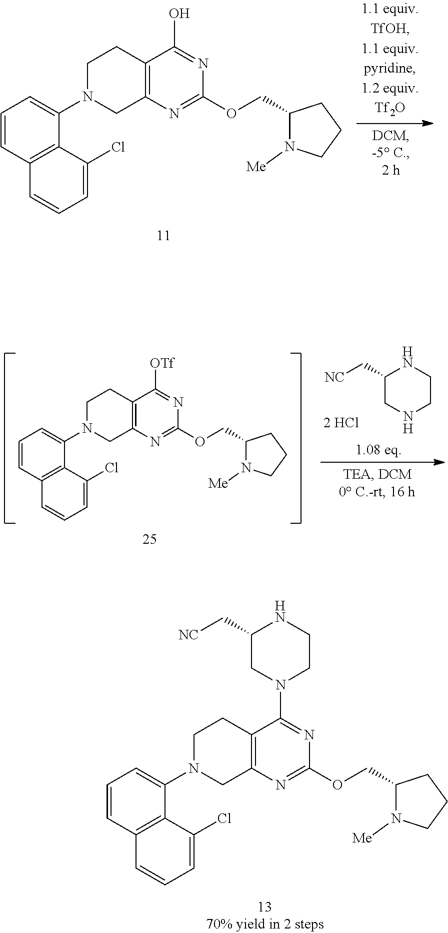

A 100-mL flask was charged with 11 (4 g, 97.1% purity), DCM (40 mL) and the resultant solution was cooled to −5° C. Pyridine (1.1 equiv. 0.8 mL) was added and then followed by slow addition of TfOH (1.1 equiv. 0.9 mL, exotherm). Tf₂O (1.2 equiv. 1.83 mL) was added slowly to the resultant mixture (exotherm!). The reaction was stirred at −5° C. for 2 h and quenched with 20 mL 5% aq. NaHCO₃ solution. The organic layer was drained into a second reactor and cooled to 5° C. Piperazine side chain (1.08 equiv., 2 g) was added followed by addition of triethylamine (3.6 equiv., 4.5 mL). The reaction mixture was stirred for 16 h at room temperature. The reaction mixture was washed with 30 mL 15% aq. Na₂CO₃ solution and the aq layer (pH=9.8) was discarded. Solvent switch from DCM to 8 mL of DMAc was carried out by distillation at 40° C. To the resultant solution in DMAc was added 6 mL water to afford a hazy mixture. The mixture was seeded and the product precipitated. The remaining 4 mL water was added to precipitate out the remaining product. The slurry was stirred for 1 h, filtered, washed with 6 mL water/DMAc (V/V=60/40), 20 mL of water sequentially. The product was dried at 40° C. in the vacuum oven to afford 13 as yellow solid (3.7 g, 92% purity, 70% yield).

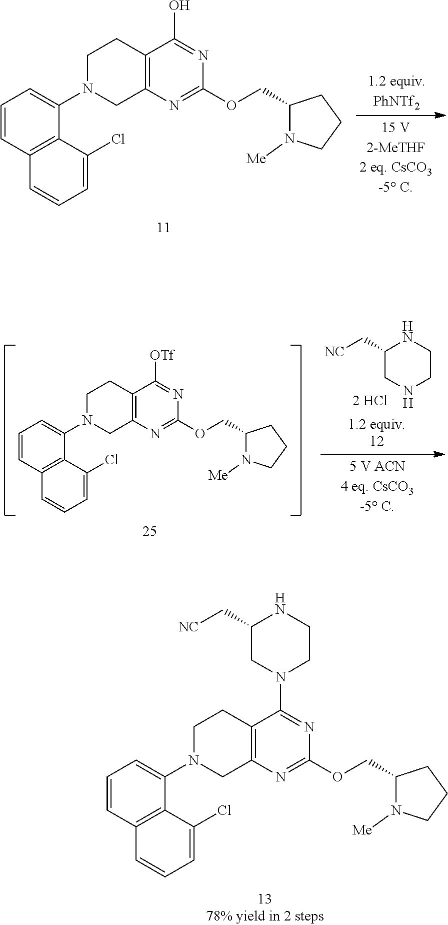

To a 500-mL flask was charged with 11 (20 g, 97.1% purity), 2-Me-THF (300 mL) and the resultant solution was cooled to −5° C. Cs₂CO₃(2 equiv., 30 g) and PhNTf₂ (1.2 equiv., 19.6 g) were added sequentially and the reaction was kept at approx. −5° C. under stirring for 2 h. Once the 11 was consumed, acetonitrile (100 mL) was added, followed by the addition of 12 (1.2 equiv., 10.8 g) and Cs₂CO₃ (4 equiv., 60 g). The reaction was stirred at −5° C. for 15 h and then H₂O (100 mL) was added to give a biphasic mixture. The aq. layer was separated and extracted with 2-Me-THF (100 mL). The combined organic layers were washed with brine (200 mL) and concentrated. To the resultant crude mixture in 2-Me-THF (200 mL) was added 2M HCl (114 mL, 5 equiv.) and H₂O (100 mL) sequentially. The organic layer was separated and treated with 2M HCl (23 mL, 1 equiv.) and H₂O (20 mL). Two aqueous layers were combined and the solution pH was adjusted to 10-12 with NaOH (10 M). The aq solution was extracted with 2-Me-THF (200 mL). The organic solution was washed by brine (200 mL) and solvent switched to acetonitrile and concentrated to a final volume of ~80 mL in acetonitrile. H₂O (100 mL) was added to afford a cloudy solution followed by seeding with 1 wt % seed. The slurry was stirred at room temperature for 15 h followed by addition of H₂O (40 mL) dropwise. The resultant slurry was filtered and the filter cake was washed with 2×40 mL ACN/H₂O=1/4 mixed solvents. The product was dried in the vacuum oven to afford 13 as a white solid (20.8 g, 91% purity, 78% yield).

### Example 5B

### 6.3 Synthesis of 13 Through 2-Nosylate:

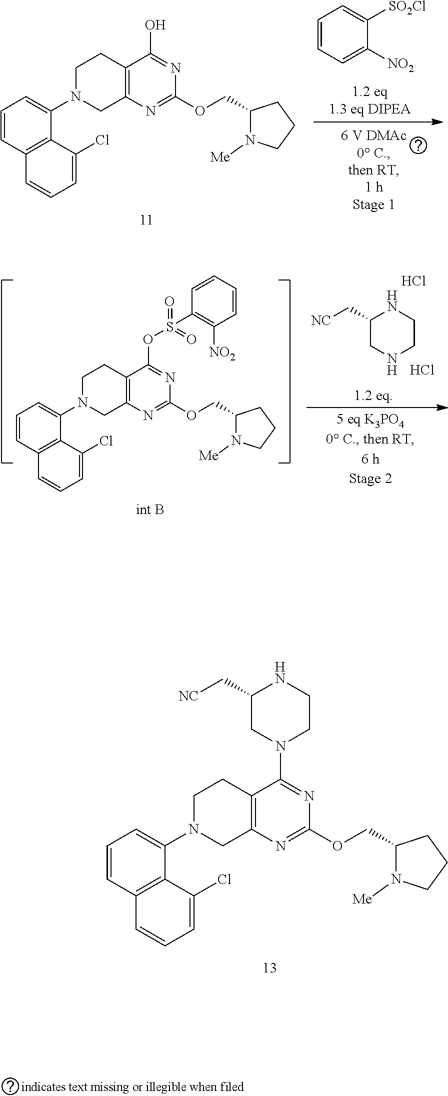

Stage 1: To a mixture of 11 (3.0 g, 97.1% assay, 6.85 mmol) and 2-NO₂PhSO₂Cl (1.8 g, 8.23 mmol) in DMAc (18 mL) at 0° C. was added DIPEA (1.6 mL, 8.91 mmol). The reaction mixture was stirred at 0° C. for 10 min, warmed to room temperature and stirred for 1 hour.

Stage 2: Cool the mixture in Stage 1 to 0° C., piperazine side chain (1.6 g, 8.23 mmol) and K₃PO₄ (7.3 g, 34.3 mmol) was added sequentially. Then, warm the reaction to room temperature. The mixture was stirred at room temperature for 6 h. To the resultant slurry was added H₂O (18 mL). Then KOH (0.77 g, 137.7 mmol) was added to the resulting mixture, keeping the mixture temperature at 35° C. until a clear biphasic solution formed. The top organic layer was separated followed by addition of H₂O (2 mL) until the mixture turned hazy. 2 wt % seeds were added to the mixture. Then, stirred the mixture at room temperature for 15 hours. H₂O (36 mL) was added slowly over 2 hours to afford a white slurry. The slurry was filtered, and the cake was washed with 9 mL DMAc/H₂O=1/3 (V/V) mixture and 6 mL H₂O sequentially. The wet cake was dried under vacuum at room temperature for 18 hours to give product 13 as a white solid (3.5 g, 91.1 wt %, 87.7% yield, 99.4% purity). ¹H NMR (400 MHz, DMSO-d6) δ 7.86 (dd, J=8.2, 1.3 Hz, 1H), 7.68 (d, J=8.1 Hz, 1H), 7.53 (dd, J=7.5, 1.3 Hz, 1H), 7.48 (t, J=7.8 Hz, 1H), 7.40 (t, J=7.8 Hz, 1H), 7.31-7.23 (m, 1H), 4.23 (dd, J=10.8, 4.8 Hz, 1H), 4.17 (dd, J=17.1, 3.2 Hz, 1H), 4.00 (ddd, J=10.4, 6.4, 3.6 Hz, 1H), 3.84 (d, J=11.7 Hz, 1H), 3.67 (d, J=16.7 Hz, 1H), 3.43 (s, 2H), 3.40 (s, 1H), 3.20-2.73 (m, 8H), 2.73-2.63 (m, 3H), 2.47 (ddd, J=8.8, 4.6, 2.3 Hz, 1H), 2.29 (d, J=1.5 Hz, 3H), 2.10 (q, J=8.4 Hz, 1H), 1.92-1.81 (m, 1H), 1.67-1.50 (m, 3H). ¹³C NMR (101 MHz, DMSO-d6) δ 165.7 (d, J=15.2 Hz), 163.9 (d, J=8.3 Hz), 162.1 (d, J=2.4 Hz), 148.1 (d, J=2.2 Hz), 137.0, 129.4, 128.9, 128.5, 126.8, 125.8, 125.0, 124.6, 118.7 (d, J=1.9 Hz), 118.7 (d, J=2.2 Hz), 108.3 (d, J=20.4 Hz), 68.8 (d, J=4.3 Hz), 63.4, 58.7 (d, J=6.7 Hz), 57.0 (d, J=3.9 Hz), 51.7 (d, J=8.5 Hz), 50.9 (d, J=41.1 Hz), 50.0 (d, J=5.5 Hz), 47.7 (d, J=25.3 Hz), 44.6 (d, J=2.9 Hz), 41.2 (d, J=1.7 Hz), 28.5 (d, J=4.5 Hz), 25.7 (d, J=8.3 Hz), 22.5, 21.2 (d, J=20.0 Hz). HRMS (ESI) calculated for C₂₉H₃₅ClN₇O: 532.2586 [M+H]⁺, Found: 532.2593.

### An Alternative Synthesis of 13 Through 2-Nosylate:

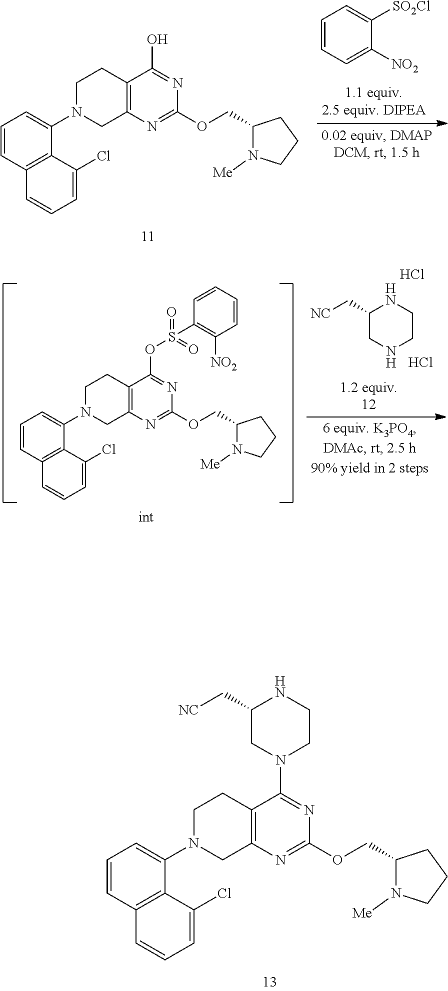

Stage 1: To a mixture of 11 (50 g, 97.1% purity) and DIPEA (2.5 equiv., 50 mL) in DCM (350 mL) at 0° C. was added 2-NO₂PhSO₂Cl (1.1 equiv., 27.8 g) and DMAP (0.05 equiv., 0.28 g) sequentially. The reaction mixture was stirred at 0° C. for 15 min, warmed to room temperature and stirred at this temperature for 1.5 h. H₂O (150 mL) was added to the reaction mixture and layers were separated. The bottom organic layer was separated and dried (MgSO₄.) and concentrated to afford crude intermediate 25, which was used directly in the next stage.

Stage 2: To a mixture of piperazine side chain (1.2 equiv., 27.0 g) and crude intermediate 25 DMAc (150 mL) at 10° C. was added K₃PO₄ (6 equiv., 145 g). The mixture was stirred at room temperature for 2.5 h. To the resultant slurry was added DMAc (250 mL) and H₂O (400 mL) sequentially, keeping the reaction temperature around 40° C. until a clear biphasic solution formed. The top organic layer was separated followed by addition of H₂O until the mixture turned hazy. 2 wt % seeds was added and the mixture was stirred at room temperature for 15 h. H₂O (640 mL) was added slowly over 6 h to afford a white slurry. The slurry was filtered and the cake was washed with 100 mL DMAc/H₂O=1/2 (V/V) mixture and 100 mL H₂O sequentially. The product was dried under vacuum at room temperature for 18 h to give 9 as a white solid (60 g, 90.1% assay, 90% yield).

13: white solid (60 g, 90.1% assay, 90% yield). R_{f}=0.15 (dichloromethane/methanol=5/1). ¹H NMR (400 MHz, DMSO-d₆) δ 7.88 (dd, J=8.3, 1.3 Hz, 1H), 7.71 (d, J=8.1 Hz, 1H), 7.55 (dd, J=7.4, 1.3 Hz, 1H), 7.50 (t, J=7.8 Hz, 1H), 7.42 (t, J=7.8 Hz, 1H), 7.29 (ddd, J=7.6, 2.9, 1.2 Hz, 1H), 4.24 (dd, J=10.8, 4.8 Hz, 1H), 4.17 (dd, J=17.2, 2.9 Hz, 1H), 4.06-3.94 (m, 1H), 3.89-3.81 (m, 1H), 3.73-3.64 (m, 1H), 3.45 (s, 1H), 3.41 (s, 2H), 3.16-2.74 (m, 8H), 2.74-2.62 (m, 3H), 2.52-2.42 (m, 1H), 2.31 (d, J=1.5 Hz, 3H), 2.12 (q, J=8.5 Hz, 1H), 1.94-1.81 (m, 1H), 1.71-1.50 (m, 3H). ¹³C NMR (101 MHz, DMSO-d6) δ 165.9 (d, J=14.7 Hz), 164.1 (d, J=8.0 Hz), 162.3 (d, J=2.5 Hz), 148.3 (d, J=2.2 Hz), 137.2, 129.7, 129.1, 128.8, 127.1, 126.1, 125.2, 124.9 (d, J=2.0 Hz), 119.0 (d, J=1.6 Hz), 118.9 (d, J=1.9 Hz), 108.5 (d, J=20.0 Hz), 69.0 (d, J=4.0 Hz), 63.7, 58.9 (d, J=6.9 Hz), 57.2 (d, J=3.8 Hz), 51.9 (d, J=8.4 Hz), 51.1 (d, J=41.1 Hz), 50.3 (d, J=5.7 Hz), 47.9 (d, J=25.3 Hz), 44.9 (d, J=2.5 Hz), 41.4 (d, J=1.7 Hz), 28.8 (d, J=4.6 Hz), 25.9 (d, J=8.4 Hz), 22.7, 21.4 (d, J=19.9 Hz). HRMS (ESI) calculated for C₂₉H₃₅ClN₇O: 532.2586 [M+H]⁺, Found: 532.2593.

### Example 6

### Reaction Procedure:

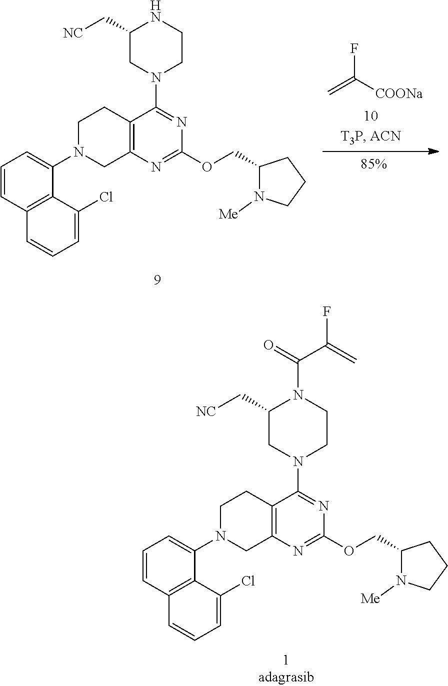

To 9 (5 g, 90.1% purity) in dry MeCN (75 mL) at 10-20° C. was added compound 10 (1.7 equiv., 1.61 g) and 50% T₃P in ethyl acetate solution (1.5 equiv., 7.5 mL) sequentially. The mixture was stirred at 10-20° C. for 2 h. 12 wt % aqueous K₂CO₃ solution was added to the reaction mixture until pH of the mixture reached to 8-9. The resultant biphasic solution was separated and the organic layer was washed with 20 wt % aq. K₃PO₄ solution. The combined aqueous phases were back extracted with 2-MeTHF (15 mL). The two organic layers were combined and solvent switched to isopropanol. The resultant mixture (40 mL) was heated to 60° C. followed by addition of n-heptane (10 mL) to afford a clear solution. Subsequently cooling of this solution to room temperature afforded a slurry. The slurry was filtered and the cake was washed with 10 mL IPA/H₂O=1/6 (V/V) and 10 mL H₂O sequentially. The product was dried under vacuum at 35° C. for 18 h to give adagrasib (1) as a white solid (4.3 g, 99% purity, 85% yield)

Adagrasib (1): white solid (4.3 g, 99% purity, 85% yield). R_{f}=0.18 (dichloromethane/methanol=10/1). ¹H NMR (400 MHz, DMSO-d6) δ 7.91 (dd, J=8.2, 1.3 Hz, 1H), 7.57 (dt, J=7.4, 1.1 Hz, 1H), 7.52 (q, J=7.7 Hz, 1H), 7.43 (t, J=7.8 Hz, 1H), 7.33 (ddd, J=15.9, 7.6, 1.2 Hz, 1H), 5.38 (dd, J=18.0, 4.1 Hz, 1H), 5.36-5.18 (m, 1H), 4.84 (s, 1H), 4.26-4.21 (m, 1H), 4.18 (dd, J=16.1, 9.5 Hz, 1H), 4.08-3.94 (m, 2H), 3.91-3.86 (m, 1H), 3.75 (dd, J=20.2, 17.4 Hz, 1H), 3.47 (q, J=7.3 Hz, 1H), 3.23 (dd, J=13.7, 3.7 Hz, 1H), 3.18-2.99 (m, 3H), 2.98-2.88 (m, 2H), 2.70-2.52 (m, 1H), 2.48-2.46 (m, 0H), 2.32 (d, J=3.5 Hz, 3H), 2.14 (qd, J=8.7, 2.2 Hz, 1H), 1.90 (dq, J=12.1, 8.2 Hz, 1H), 1.71-1.50 (m, 3H). ¹³C NMR (101 MHz, DMSO-d₆) δ 165.9, 164.3 (d, J=14.5 Hz), 162.1 (d, J=2.1 Hz), 160.8 (dd, J=31.7, 12.2 Hz), 155.6 (d, J=266.7 Hz), 148.0 (d, J=20.4 Hz), 137.0 (d, J=2.9 Hz), 129.4 (d, J=2.1 Hz), 128.9 (d, J=4.3 Hz), 128.5, 126.8 (d, J=2.6 Hz), 125.8, 125.0 (d, J=5.6 Hz), 124.7 (d, J=6.1 Hz), 118.7 (d, J=2.6 Hz), 118.1 (d, J=3.9 Hz), 108.7 (d, J=20.7 Hz), 99.9 (d, J=14.3 Hz), 68.9 (d, J=7.0 Hz), 63.4 (d, J=2.7 Hz), 58.5 (d, J=25.8 Hz), 57.0 (d, J=4.0 Hz), 50.0, 49.0, 48.2, 47.3 (d, J=36.2 Hz), 46.4, 41.2 (d, J=2.9 Hz), 28.5 (d, J=7.1 Hz), 25.2 (d, J=35.0 Hz), 22.5, 18.1. ¹⁹F NMR (101 MHz, DMSO-d6) δ −105.18 (d, J=334.7 Hz). HRMS (ESI) calcd for C₃₂H₃₆ClFN₇O₂: 604.2598 [M+H]⁺, found 604.2607.

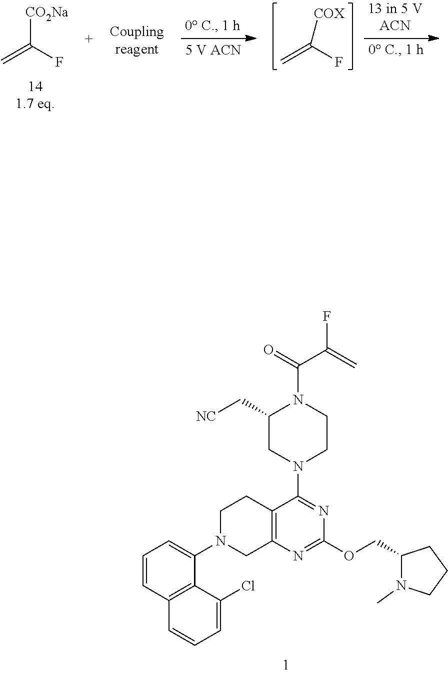

| Entries | 1 | 2 | 3 | 4 | 5 | 6 | 7 |
|---|---|---|---|---|---|---|---|
| Coupling | 1.5 eq | 1.5 eq | 0.5 eq | 1.5 eq | 1.5 eq | 1.2 eq | 1.4 eq |
| reagent | MsCl | TsCl | 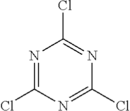 | 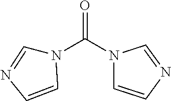 [SMILES: O=C(n1ccnc1)n1ccnc1] | 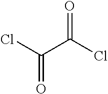 | 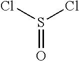 | 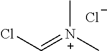 |
| | | | TCT | CDI | | | |
| Reaction | 0%/94.2% | 0%/90.4% | 22.5%/63.5% | 34%/0% | 0.1%/98.5% | 4.7%/90.6% | 0.2%/98.7% |
| profile | | | | | | | |
| (13/1) | | | | | | | |

### Reaction Setup:

To a mixture of 14 (1.76 g, 1.7 equiv.) and DMF (0.1 equiv.) in acetonitrile (25 mL) at 0° C. was added a coupling reagent. The reaction mixture was stirred at 0° C. for 1 h and then a solution of 13 (5.0 g, 9.26 mmol) in 25 mL acetonitrile was added in 10 min.

### Workup:

Upon reaction completion, 20 wt % K3PO4 (8V) was added to give a clear biphasic solution (pH=12). The bottom aqueous layer was discarded and the organic layer was concentrated to 15 mL. 60 mL 2-MeTHF was added to the mixture and concentrated to 15 mL to remove the residue acetonitrile. The azeotrope process was repeated twice and the resulting mixture was added with 45 mL 2-MeTHF. The mixture was washed by 25 mL H₂O and the result organic layer was concentrated to 15 mL. 50 mL isopropanol was added to the mixture and concentrated to 15 mL to remove the residue 2-MeTHF. The azeotrope process was repeated twice and the resulting mixture was added 35 mL isopropanol. The mixture was warmed to 55° C. and was added with 1% activated carbon (Darco G-60). Filtration after stirring at 55° C. for 20 min afforded a clear solution. 10 mL n-heptane was added and the mixture was cooled to 45° C. in 2 hours. 2% seeds were charged and maintain the temperature at 45° C. for 2 hours and then cool it to 35° C. in 3 hours. Maintain the temperature at 35° C. for 4 hours and then cool it to 25° C. in 3 hours. Maintain the temperature at 25° C. for 6 hours and then cool it to 0° C. in 8 hours. The result slurry was filtered and the cake was rinsed with mixed solution IPA/Heptane(v/v=3/2) twice (10 mL*2). The solid was dried under vacuum at 40° C. to give the product 1 as a white solid (in 79% yield for entry 7).

### Synthesis by T₃P:

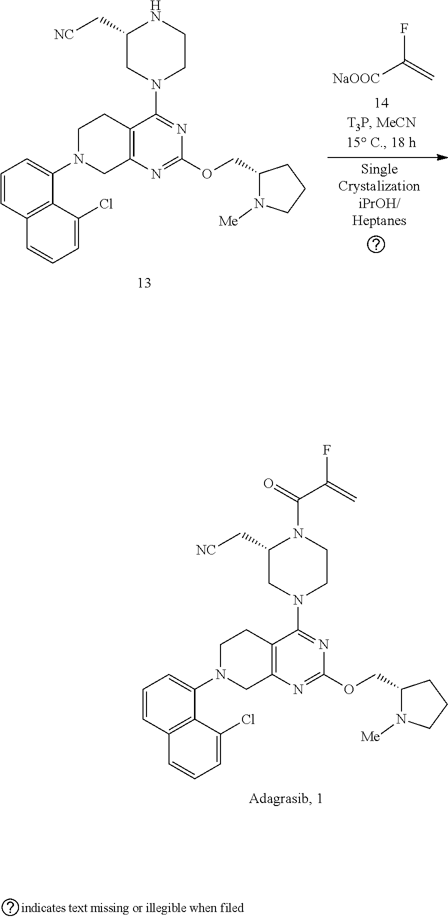

To a suspension of 14 (9.8 g, 87.99 mmol) in dry MeCN (90 mL), 50% T3P in ethyl acetate solution (462 mL, 77.64 mmol) was added in 400 mL EazyMax reactor at 20° C. over 20 min and stirred 16 h. To this mixture, a solution of 13 (30 g, 51.76 mmol) in MeCN (110 mL) was prepared by azeodrying with MeCN (300 mL×2; KF=680 ppm) was charged over 2 minutes and stirred for 3 h. After consumption of 13, aqueous K₂CO₃ solution (12 wt %, 5.0 w/w) was charged over 30 minutes. The organic layer was separated and washed with aqueous K₃PO₄ (20 wt %, 3.75 w/w). The organic layer was concentrated till 3.5V left at NMT 40° C. 2-MeTHF (6.5V) was added to mixture and concentrated till 3.5V left twice. Additional 2-MeTHF (6.5V) was added to mixture and washed 5% brine solution (2×150 mL). The organic phase was collected and concentrated till 3.5V left. The IPA (8V) was charged and concentrated till 3.5V left twice. Additional IPA was added to make 8V solution and heated to 60° C. The n-Heptane (2V) was charged to crude solution in IPA while maintaining temperature NLT 58° C. and the mixture was stirred at same temperature for 1 h and then cooled to 45° C. over 3 h. The seeds (4 w/w %) were charged as a slurry in IPA/heptane and mixture was stirred additional for 3 h at 45° C. The mixture was cooled to 35° C. over 5 h and stirred for additional 5 h. Then mixture was cooled to 22° C. over 3 h and stirred for additional 6 h. The slurry was wet-milled with homogenizer for 2 h at 22° C., then stirred for additional 1 h at same temperature. The mixture was cooled to 3° C. over 8 h and stirred for additional 8 h. The solids were filtered and washed with IPA/heptane mixture (2:1; 2×2V), vacuum dried for 2 h and then oven dried for 60 h at 35° C. to provide off-white 1 with 89% yield (28.15 g, 98.7 wt %, 99.7% purity). ¹H NMR (400 MHz, DMSO-d₆) δ 7.91 (dd, J=8.2, 1.3 Hz, 1H), 7.57 (dt, J=7.4, 1.1 Hz, 1H), 7.52 (q, J=7.7 Hz, 1H), 7.43 (t, J=7.8 Hz, 1H), 7.33 (ddd, J=15.9, 7.6, 1.2 Hz, 1H), 5.38 (dd, J=18.0, 4.1 Hz, 1H), 5.36-5.18 (m, 1H), 4.84 (s, 1H), 4.26-4.21 (m, 1H), 4.18 (dd, J=16.1, 9.5 Hz, 1H), 4.08-3.94 (m, 2H), 3.91-3.86 (m, 1H), 3.75 (dd, J=20.2, 17.4 Hz, 1H), 3.47 (q, J=7.3 Hz, 1H), 3.23 (dd, J=13.7, 3.7 Hz, 1H), 3.18-2.99 (m, 3H), 2.98-2.88 (m, 2H), 2.70-2.52 (m, 1H), 2.48-2.46 (m, 1H), 2.32 (d, J=3.5 Hz, 3H), 2.14 (qd, J=8.7, 2.2 Hz, 1H), 1.90 (dq, J=12.1, 8.2 Hz, 1H), 1.71-1.50 (m, 3H) ppm. ¹³C NMR (101 MHz, DMSO-d₆) δ 165.9, 164.3 (d, J=14.5 Hz), 162.1 (d, J=2.1 Hz), 160.8 (dd, J=31.7, 12.2 Hz), 155.6 (d, J=266.7 Hz), 148.0 (d, J=20.4 Hz), 137.0 (d, J=2.9 Hz), 129.4 (d, J=2.1 Hz), 128.9 (d, J=4.3 Hz), 128.5, 126.8 (d, J=2.6 Hz), 125.8, 125.0 (d, J=5.6 Hz), 124.7 (d, J=6.1 Hz), 118.7 (d, J=2.6 Hz), 118.1 (d, J=3.9 Hz), 108.7 (d, J=20.7 Hz), 99.9 (d, J=14.3 Hz), 68.9 (d, J=7.0 Hz), 63.4 (d, J=2.7 Hz), 58.5 (d, J=25.8 Hz), 57.0 (d, J=4.0 Hz), 50.0, 49.0, 48.2, 47.3 (d, J=36.2 Hz), 46.4, 41.2 (d, J=2.9 Hz), 28.5 (d, J=7.1 Hz), 25.2 (d, J=35.0 Hz), 22.5, 18.1 ppm. ¹⁹F NMR (101 MHz, DMSO-d₆) δ −105.18 (d, J=334.7 Hz) ppm. HRMS (ESI) calcd for C₃₂H₃₆ClFN₇O₂: 604.2598 [M+H]⁺, found 604.2607.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A method of synthesizing adagrasib, comprising step (a) of reacting a compound of the following structure: 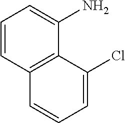 [SMILES: Nc1cccc2cccc(Cl)c12]
with a 4-halobutyrate, a aprotic solvent, an iodide, and a base to produce a final compound of step (a) with the following structure:

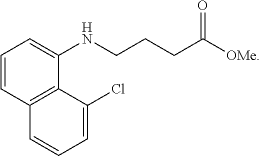 [SMILES: COC(=O)CCCNc1cccc2cccc(Cl)c12]

2-9. (canceled)

10. The method of claim 1, wherein the method further comprises step (b):
b) reacting the final compound of step (a) with a 2-halo-N-methoxy-N-methylacetamide, an aprotic solvent and a base to produce a final compound of step (b) with the following structure:

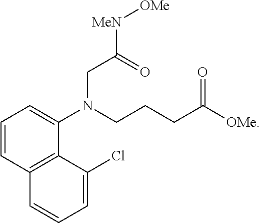 [SMILES: COC(=O)CCCN(CC(=O)N(C)OC)c1cccc2cccc(Cl)c12]

11-19. (canceled)

20. The method of claim 10, wherein the method further comprises step (c):
c) reacting the final compound of step (b) with a base and an aprotic solvent to produce a final compound of step (c) with the following structure:

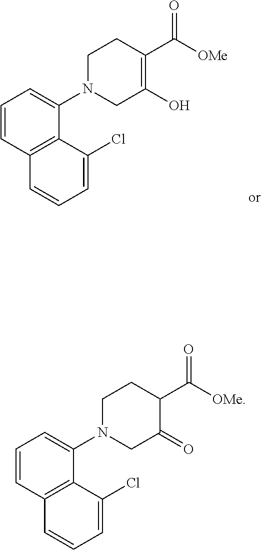 [SMILES: COC(=O)C1=C(O)CN(c2cccc3cccc(Cl)c23)CC1 ; COC(=O)C1CCN(c2cccc3cccc(Cl)c23)CC1=O]

21-23. (canceled)

24. The method of claim 20, wherein the method further comprises step (d):
d) reacting the final compound of step (c) with an S-alkylated isothiourea salt, a solvent and a base to produce a final compound of step (d) with the following structure:

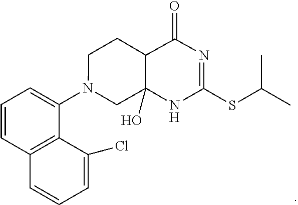 [SMILES: CC(C)SC1=NC(=O)C2CCN(c3cccc4cccc(Cl)c34)CC2(O)N1]

25-32. (canceled)

33. The method of claim 24, wherein the method further comprises step (e):
e) reacting the final compound of step (d) with an acid, or a mesylating/triflating agent, and an aprotic solvent to produce a final compound of step (e) with the following structure:

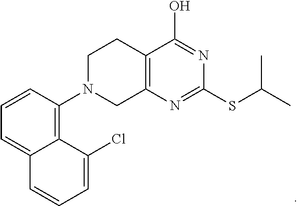 [SMILES: CC(C)Sc1nc(O)c2c(n1)CN(c1cccc3cccc(Cl)c13)CC2]

34-37. (canceled)

38. The method of claim 33, wherein the method further comprises step (f):
f) reacting the final compound of step (e) with an oxidizing agent, a base and/or an alkoxide, a polar solvent, and, optionally, a catalyst to produce a final compound of step (f) with the following structure:

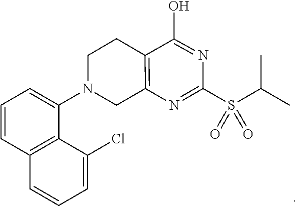 [SMILES: CC(C)S(=O)(=O)c1nc(O)c2c(n1)CN(c1cccc3cccc(Cl)c13)CC2]

39-46. (canceled)

47. The method of claim 38, wherein the method further comprises step (g):
g) reacting the final compound of step (f) with an alkoxide and a polar aprotic solvent to produce a final compound of step (g) with the following structure:

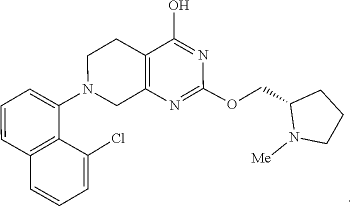 [SMILES: CN1CCC[C@H]1COc1nc(O)c2c(n1)CN(c1cccc3cccc(Cl)c13)CC2]

48-50. (canceled)

51. The method of claim 47, wherein the method further comprises step (h):
h) reacting the final compound of step (g) with an activating agent, a base, a polar aprotic solvent, and an additive, to produce a final compound of step (h) with the following structure: 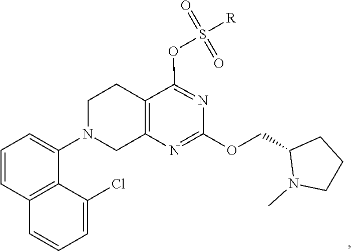
wherein R is selected from the group consisting of a substituted phenyl, methyl, ethyl, propyl, C₄F₉, CF₃ and toluene.

52-59. (canceled)

60. The method of claim 51, wherein the method further comprises step (i):
i) reacting the final compound of step (h) with a salt of 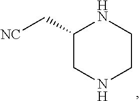
a base and a polar aprotic solvent to produce a final compound of step (i) with the following structure:

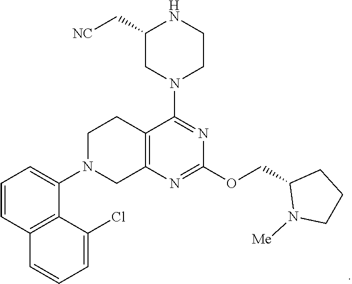 [SMILES: CN1CCC[C@H]1COc1nc2c(c(N3CCN[C@@H](CC#N)C3)n1)CCN(c1cccc3cccc(Cl)c13)C2]

61-75. (canceled)

76. The method of claim 20, wherein the method further comprises step (d′):
d′) reacting the final compound of step (c) with an alkoxide and a polar solvent to produce a final compound of step (d′) with the following structure:

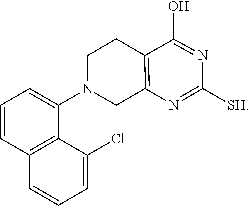 [SMILES: Oc1nc(S)nc2c1CCN(c1cccc3cccc(Cl)c13)C2]

77-79. (canceled)

80. The method of claim 76, wherein the method further comprises step (e′):
e′) reacting the final compound of step (d′) with an alkylating agent, an inorganic base and/or an alkoxide, and a polar solvent to produce a final compound of step (e′) with the following structure:

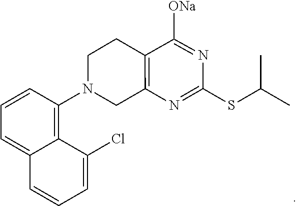

81-85. (canceled)

86. The method of claim 80, wherein the method further comprises step (f′):
f) reacting the final compound of step (e′) with an oxidizing agent, a base and/or an alkoxide, a polar solvent and, optionally, a catalyst to produce a final compound of step (f′) with the following structure:

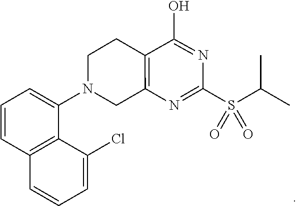 [SMILES: CC(C)S(=O)(=O)c1nc(O)c2c(n1)CN(c1cccc3cccc(Cl)c13)CC2]

87-94. (canceled)

95. The method of claim 20, wherein the method further comprises step (d′″):
d′″) reacting the final compound of step (c) with an S-alkylated isothiourea salt, a solvent and a base to produce a final compound of step (d′″) with the following structure:

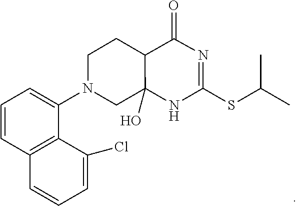 [SMILES: CC(C)SC1=NC(=O)C2CCN(c3cccc4cccc(Cl)c34)CC2(O)N1]

96-103. (canceled)

104. The method of claim 95, wherein the method further comprises step (e′″):
e′″) reacting the final compound of step (d′″) with a water miscible solvent and a base to produce a final compound of step (e) with the following structure:

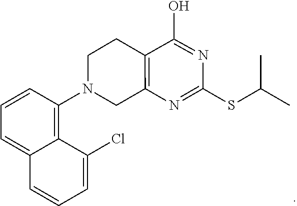 [SMILES: CC(C)Sc1nc(O)c2c(n1)CN(c1cccc3cccc(Cl)c13)CC2]

105-110. (canceled)

111. The method of claim 76, further comprising step (e″):
(e″) reacting the final compound of step (d′) with a phosgene or a phosgene derivative, a polar aprotic solvent and optionally, a mineral acid to produce a final compound of step (e″) with the following structure:

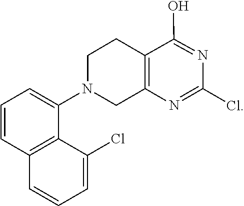 [SMILES: Oc1nc(Cl)nc2c1CCN(c1cccc3cccc(Cl)c13)C2]

112-115. (canceled)

116. The method of claim 111, further comprising step (f″):
(f″) reacting the final compound of step (e″) with an alkoxide and a polar aprotic solvent to produce a final compound of step (f″) with the following structure:

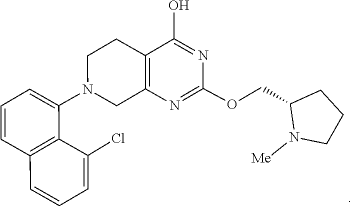 [SMILES: CN1CCC[C@H]1COc1nc(O)c2c(n1)CN(c1cccc3cccc(Cl)c13)CC2]

117-119. (canceled)

120. The method of claim 47, further comprising step (h′):
(h′) reacting the final compound of step (g) with a triflating agent, an acid, a polar aprotic solvent, an additive, and optionally a base, to produce a final compound of step (h′) with the following structure:

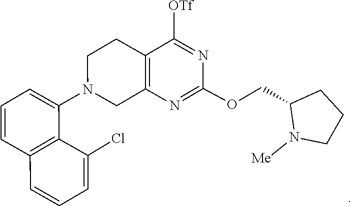 [SMILES: CN1CCC[C@H]1COc1nc2c(c(OS(=O)(=O)C(F)(F)F)n1)CCN(c1cccc3cccc(Cl)c13)C2]

121-129. (canceled)

130. The method of claim 120, wherein the method further comprises step (i′):
i′) reacting the final compound of step (h′) with a salt of 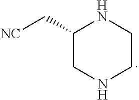
a base, and a polar aprotic solvent to produce a final compound of step (i′) with the following structure:

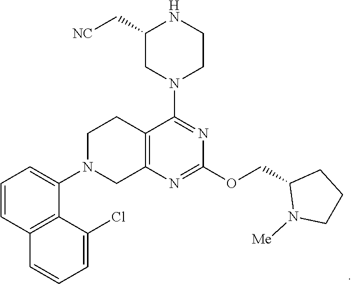 [SMILES: CN1CCC[C@H]1COc1nc2c(c(N3CCN[C@@H](CC#N)C3)n1)CCN(c1cccc3cccc(Cl)c13)C2]

131-137. (canceled)

138. The method of claim 47, wherein the method further comprises step (h″):
(h″) reacting the final compound of step (g) with a triflating agent, an acid, a polar aprotic solvent, an additive, and optionally a base, to produce a final compound of step (h″) with the following structure:

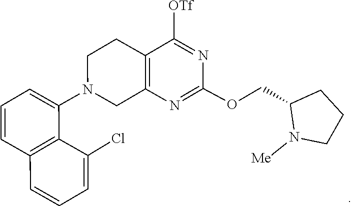 [SMILES: CN1CCC[C@H]1COc1nc2c(c(OS(=O)(=O)C(F)(F)F)n1)CCN(c1cccc3cccc(Cl)c13)C2]

139-147. (canceled)

148. The method of claim 138, further comprising step (i″):
(i″): reacting the final compound of step (h″) with a salt of 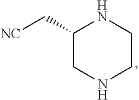
a base and a polar aprotic solvent to produce a final compound of step (i″) with the following structure:

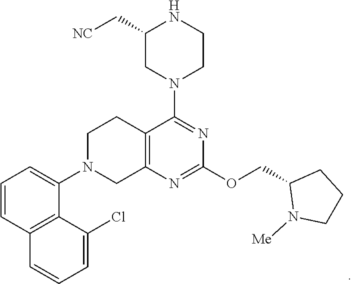 [SMILES: CN1CCC[C@H]1COc1nc2c(c(N3CCN[C@@H](CC#N)C3)n1)CCN(c1cccc3cccc(Cl)c13)C2]

149-155. (canceled)

156. The method of claim 47, wherein the method further comprises step (h′″):
(h′″) reacting the final compound of step (g) with an aryl- or heteroaryl sulfonyl, a base, a polar aprotic solvent, and an additive, to produce a final compound of step (h′″) with the following structure:

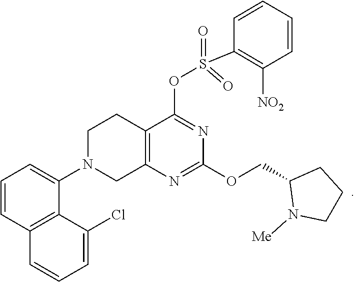 [SMILES: CN1CCC[C@H]1COc1nc2c(c(OS(=O)(=O)c3ccccc3[N+](=O)[O-])n1)CCN(c1cccc3cccc(Cl)c13)C2]

157-169. (canceled)

170. The method of claim 156, further comprising step (i′″):
(i′″) reacting the final compound of step (h′″) with a salt of 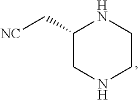
a base and a polar aprotic solvent to produce a final compound of step (i′″) with the following structure:

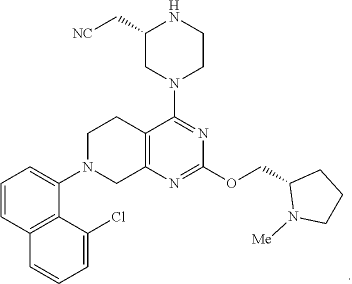 [SMILES: CN1CCC[C@H]1COc1nc2c(c(N3CCN[C@@H](CC#N)C3)n1)CCN(c1cccc3cccc(Cl)c13)C2]

171-186. (canceled)

187. A compound selected from the following structures: 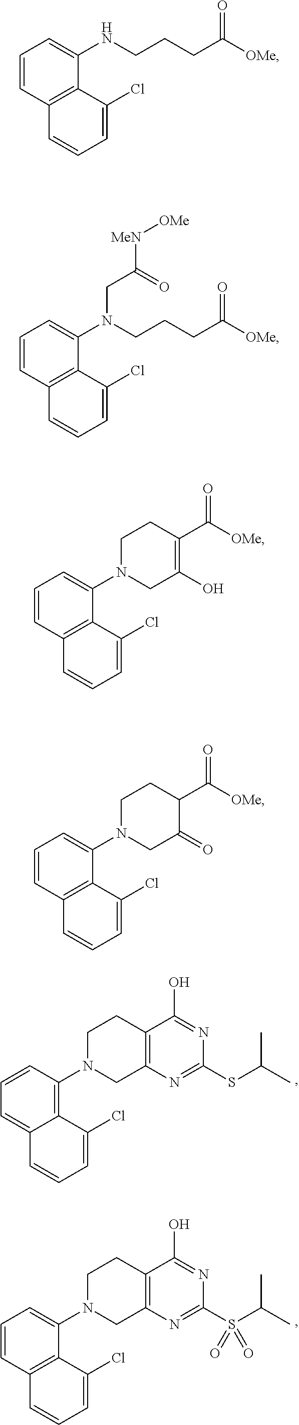 [SMILES: COC(=O)CCCNc1cccc2cccc(Cl)c12 ; COC(=O)CCCN(CC(=O)N(C)OC)c1cccc2cccc(Cl)c12 ; COC(=O)C1=C(O)CN(c2cccc3cccc(Cl)c23)CC1 ; COC(=O)C1CCN(c2cccc3cccc(Cl)c23)CC1=O ; CC(C)Sc1nc(O)c2c(n1)CN(c1cccc3cccc(Cl)c13)CC2 ; CC(C)S(=O)(=O)c1nc(O)c2c(n1)CN(c1cccc3cccc(Cl)c13)CC2] 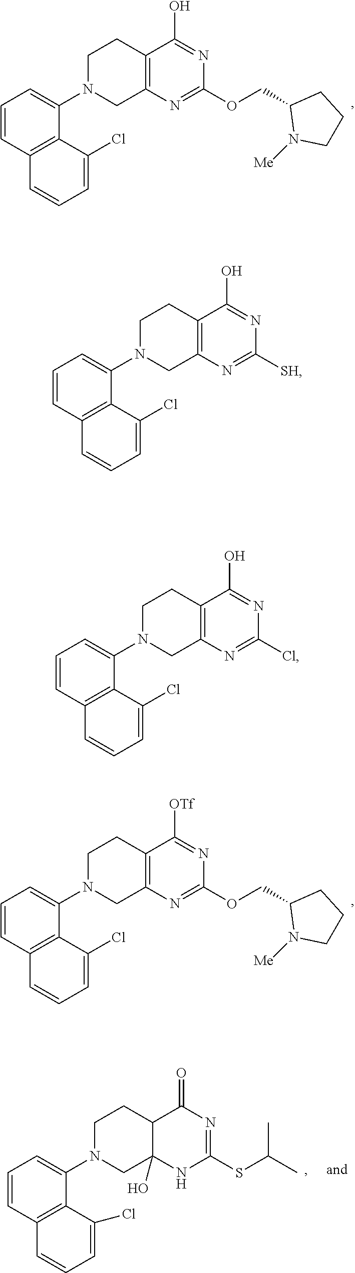 [SMILES: CN1CCC[C@H]1COc1nc(O)c2c(n1)CN(c1cccc3cccc(Cl)c13)CC2 ; Oc1nc(S)nc2c1CCN(c1cccc3cccc(Cl)c13)C2 ; Oc1nc(Cl)nc2c1CCN(c1cccc3cccc(Cl)c13)C2 ; CN1CCC[C@H]1COc1nc2c(c(OS(=O)(=O)C(F)(F)F)n1)CCN(c1cccc3cccc(Cl)c13)C2 ; CC(C)SC1=NC(=O)C2CCN(c3cccc4cccc(Cl)c34)CC2(O)N1] 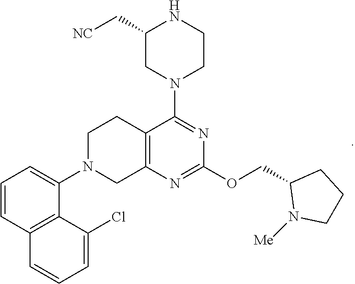 [SMILES: CN1CCC[C@H]1COc1nc2c(c(N3CCN[C@@H](CC#N)C3)n1)CCN(c1cccc3cccc(Cl)c13)C2]
